SEQUENCE LISTING (230 sequences)

SEQ ID NO: 1 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 2 - protein, 188 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 3 - protein, 132 aa, Bacteriophage Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 4 - protein, 329 aa, Bacteriophage Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 5 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 6 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 7 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 8 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 9 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 10 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 11 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 12 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 13 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 14 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 15 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 16 - protein, 132 aa, Bacteriophage Qbeta
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Bacteriophage Qbeta
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Bacteriophage Qbeta
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Bacteriophage Qbeta
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 132 aa, Bacteriophage Qbeta
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 21 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 22 - protein, 131 aa, Bacteriophage AP205
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 23 - protein, 131 aa, Bacteriophage AP205
MANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 24 - DNA, 37 bp, artificial sequence
  PCR primer
atatatgctagccccttacacctaccagagtgatttg

SEQ ID NO: 25 - DNA, 37 bp, artificial sequence
  PCR primer
atatatctcgagtgatatctggaagtctgtcatagag

SEQ ID NO: 26 - DNA, 37 bp, artificial sequence
  PCR primer
atatatgctagcccccattagacagctgcactacagg

SEQ ID NO: 27 - DNA, 37 bp, artificial sequence
  PCR primer
atatatctcgagggaagacacagattccatggtgaag

SEQ ID NO: 28 - protein, 7 aa, artificial sequence
  synthesized sequence
GGGGGCG

SEQ ID NO: 29 - DNA, 5473 bp, artificial sequence
  Cloning Vector
acatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggatccacaccaccaccaccaccacggttctggtgacgacgatgacaaagcgctagccctcgagggtggtggtggtggttgcggttaataagtttaaacgcggccgcatgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcg

SEQ ID NO: 30 - DNA, 43 bp, artificial sequence
  PCR primer
tatggatccggctagcgctcgagggtttaaacggcggccgcat

SEQ ID NO: 31 - DNA, 45 bp, artificial sequence
  PCR primer
tcgaatgcggccgccgtttaaaccctcgagcgctagccggatcca

SEQ ID NO: 32 - DNA, 58 bp, artificial sequence
  PCT primer
gatccacaccaccaccaccaccacggttctggtgacgacgatgacaaagcgctagccc

SEQ ID NO: 33 - DNA, 58 bp, artificial sequence
  PCR primer
tcgagggctagcgctttgtcatcgtcgtcaccagaaccgtggtggtggtggtggtgtg

SEQ ID NO: 34 - DNA, 42 bp, artificial sequence
  PCR primer
tcgagggtggtggtggtggttgcggttaataagtttaaacgc

SEQ ID NO: 35 - DNA, 42 bp, artificial sequence
  PCR primer
ggccgcgtttaaacttattaaccgcaaccaccaccaccaccc

SEQ ID NO: 36 - protein, 271 aa, Homo sapiens [sequence omitted]

SEQ ID NO: 37 - protein, 271 aa, Macaca mulatta
MAKVPDMFEDLKNCYSENEEDSSSIDHLSLNQKSFYDVSYGPLHEGCMDQSVSLSISEISKTSKLTFKQSMVVVSTNGKVLKKRRLSLSQSITDNNLEAIANDSEEEIIKPRSAPFSFLSNMTYHFIRIIKHEFILNDTLNQTIIRANDQHLTAAAIHNLDEAVKFDMGAYTSSKDDTKVPVILRISKTQLYVSAQDEDQPVLLKEMPEINKTITGSETNFLFFWETHGTKNYFISVAHPNLFIATKHDNWVCLAKGLPSITDFQILENQA

SEQ ID NO: 38 - protein, 270 aa, Equus caballus
MAKVPDLFEDLKNCYSENEDYSSEIDHLSLTQKSFYDASYDPLPEDCMDTFMSLSTSETSKTSKLNFKESVVLVAANGKTLKKRRLSLNQFITNDDLEAIANDPEEGIIRPRSVHYNFQSNTKYNFMRIVNHQCTLNDALNQSVIRDTSGQYLATAALNNLDDAVKFDMGAYTSEEDSQLPVTLRISKTRLFVSAQNEDEPVLLKEMPDTPKTIKDETNLLFFWERHGSKNYFKSVAHPKLFIATKQGKLVHMARGQPSITDFQILDNQF

SEQ ID NO: 39 - protein, 268 aa, Ovis aries
MAKVPDLFEDLKNCYSENEDYSSEIDHLSLNQKSFYDASYEPLREDHMNKFMSLDTSETSKTSRLSFKENVVMMTANGKILKKRRLSLNQFITDDDLEAIANDTEEEIIKPRSAHYSFQSNVKYNFMRVIHQECILNDALNQSIIRDMSGPYLTAATLNNLEEAVKFDMVAYVSEEDSQLPVTLRISKTQLFVSAQNEDEPVLLKEMPETPKIIKDETNLLFFWEKHGSMDYFKSVAHPKLFIATKQEKLVHMASGPPSITDFQILEK

SEQ ID NO: 40 - protein, 270 aa, Felis catus
MAKVPDLFEDLKNCYSENEEYSSEIDHLTLNQKSFYDASYDPLHEDCTDKFMSPSTSETSKTPQLTLKKSVVMVAANGKILKKRRLSLNQFLTADDLEAIANEVEEEIMKPRSVAPNFYSSEKYNYQKIIKSQFILNDNLSQSVIRKAGGKYLAAAALQNLDDAVKFDMGAYTSKEDSKLPVTLRISKTRLFVSAQNEDEPVLLKEMPETPKTIRDETNLLFFWERHGSKNYFKSVAHPKLFIATQEEQLVHMARGLPSVTDFQILETQS

SEQ ID NO: 41 - protein, 268 aa, Bos taurus
MAKVPDLFEDLKNCYSENEDYSSEIDHLSLNQKSFYDASYEPLREDQMNKFMSLDTSETSKTSKLSFKENVVMVAASGKILKKRRLSLNQFITDDDLEAIANNTEEEIIKPRSAHYSFQSNVKYNFMRVIHQECILNDALNQSIIRDMSGPYLTATTLNNLEEAVKFDMVAYVSEEDSQLPVTLRISKTQLFVSAQNEDEPVLLKEMPETPKIIKDETNLLFFWEKHGSMDYFKSVAHPKLFIATKQEKLVHMASGPPSITDFQILEK

SEQ ID NO: 42 - protein, 270 aa, Sus scrofa
MAKVPDLFEDLKNCYSENEEYSSDIDHLSLNQKSFYDASYEPLPGDGMDKFMPLSTSKTSKTSRLNFKDSVVMAAANGKILKKRRLSLNQFITDDDLEAIANDTEEEIIKPRSATYSFQSNMKYNFMRVINHQCILNDARNQSIIRDPSGQYLMAAVLNNLDEAVKFDMAAYTSNDDSQLPVTLRISETRLFVSAQNEDEPVLLKELPETPKTIKDETSLLFFWEKHGNMDYFKSAAHPKLFIATRQEKLVHMAPGLPSVTDFQILENQS

SEQ ID NO: 43 - protein, 268 aa, Oryctolagus cuniculus
MAKVPDLFEDLKNCFSENEEYSSAIDHLSLNQKSFYDASYEPLHEDCMNKVVSLSTSETSVSPNLTFQENVVAVTASGKILKKRRLSLNQPITDVDLETNVSDPEEGIIKPRSVPYTFQRNMRYKYLRIIKQEFTLNDALNQSLVRDTSDQYLRAAPLQNLGDAVKFDMGVYMTSKEDSILPVTLRISQTPLFVSAQNEDEPVLLKEMPETPRIITDSESDILFFWETQGNKNYFKSAANPQLFIATKPEHLVHMARGLPSMTDFQIS

SEQ ID NO: 44 - protein, 265 aa, Canis familiaris
MAKVPDLFEDLKNCYSENEEYSSEIDHLSLNQKSFYDMSCDPLHEDCMSLSTSEISKTSQLTFKENVVVVAANGKILKKRRLSLSQFITDDDLEGIANDTEEVIMKPRSVAYNFHNNEKYNYIRIIKSQFILNDNLNQSIVRQTGGNYLMTAALQNLDDAVKFDMGAYTSEDSKLPVTLRISKTRLFVSAQNEDEPVLLKEMPETPKTIRDETNLLFFWERHGSKHYFKSVAQPKLFIATQERKLVHMARGQPSITDFRLLETQP

SEQ ID NO: 45 - protein, 270 aa, Mus musculus
MAKVPDLFEDLKNCYSENEDYSSAIDHLSLNQKSFYDASYGSLHETCTDQFVSLRTSETSKMSNFTFKESRVTVSATSSNGKILKKRRLSFSETFTEDDLQSITHDLEETIQPRSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 46 - protein, 270 aa, Rattus norvegicus
MAKVPDLFEDLKNCYSENEEYSSAIDHLSLNQKSFYDASYGSLHENCTDKFVSLRTSETSKMSTFTFKESRVVVSATSNKGKILKKRRLSFNQPFTEDDLEAIAHDLEETIQPRSAPHSFQNNLRYKLIRIVKQEFIMNDSLNQNIYVDMDRIHLKAASLNDLQLEVKFDMYAYSSGGDDSKYPVTLKVSNTQLFVSAQGEDKPVLLKEIPETPKLITGSETDLIFFWEKINSKNYFTSAAFPELLIATKEQSQVHLARGLPSMIDFQIS

SEQ ID NO: 47 - protein, 152 aa, Gorilla gorilla
KNCYSENEEDSSSIDHLSLNQKSFYHVTYGPLHEGCMDQSVSLSISETSKTSKLTFKESMVVVATNGKVLKKRRLSLSQSITDDDLEAIANDSEEEIIKPRSAPFSFLSNVKYNFMRIIKNEFILNDALNQSIIRANDQYLTAAALHNLDEA

SEQ ID NO: 48 - protein, 204 aa, Cavia porcellus
FEDLKNCYSENEEYASAIDHLSLNQKSFYDTNYDPLHENRVDEPVSPNPYENSEESNFTLEDSSDSSAVVLTSAHGEVLKKRRLSLNQTMSNEDLEAIANDSEEEIIEPWSVPYSFQSNLKFKYQRSIKKGAVITDAMHQSLIRESNGQHLKAMHVVDRKHEVKFDIDGYVSTATRIRPVTLKISKTQLYVCAQEEGQPVLLKE

SEQ ID NO: 49 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 50 - protein, 193 aa, Pan troglodytes
MLVPCPQTFQENDLSTFFPFIFEEEPIFFDTWENEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 51 - protein, 269 aa, Macaca mulatta
MAEVPELASEMMAYYSGNEDDLFFDVDGPKQMKCSFQDLDLCPLGGGIQLQISHEHYNEGFRQAVSVVVAMEKLRKMLVPCPQIFQDNDLSTLIPFIFEEEPVFLDTRNNDACVHDAPVRSLHCTLRDAQLKSLVMSGPYELKALHLQGQDLEQQVVFSMSFVQGEESNDKIPVALGLKAKNLYLSCVLKDDKPTLQLESVDPKNYPKKKMEKRFVFNKIEINNKLEFESAQFPNWYISTSQAENMPVFLGGTRGGQDITDFTMQFVSS

SEQ ID NO: 52 - protein, 268 aa, Oryctolagus cunicullus
MATVPELTSEMMAYHSGNENDLFFEADGPNYMKSCFQDLDLCCPDEGIQLRISCQPYNKSFRQVLSVVVALEKLRQKAVPCPQAFQDDGLRTFFSLIFEEEPVLCNTWDDYSLECDAVRSLHCRLQDAQQKSLVLSGTYELKALHLNAENLNQQVVFSMSFVQGEESNDKIPVALGLRGKNLYLSCVMKDDKPTLQLESVDPNRYPKKKMEKRFVFNKIEIKDKLEFESAQFPNWYISTSQTEYMPVFLGNNSGGQDLIDFSMEFVSS

SEQ ID NO: 53 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 54 - protein, 268 aa, Rattus norvegicus
MATVPELNCEIAAFDSEENDLFFEADRPQKIKDCFQALDLGCPDESIQLQISQQHLDKSFRKAVSLIVAVEKLWQLPMSCPWSFQDEDPSTFFSFIFEEEPVLCDSWDDDDLLVCDVPIRQLHCRLRDEQQKCLVLSDPCELKALHLNGQNISQQVVFSMSFVQGETSNDKIPVALGLKGLNLYLSCVMKDGTPTLQLESVDPKQYPKKKMEKRFVFNKIEVKTKVEFESAQFPNWYISTSQAEHRPVFLGNSNGRDIVDFTMEPVSS

SEQ ID NO: 55 - protein, 268 aa, Equus caballus
MAAVPDTSDMMTYCSGNENDLFFEEDGPKQMKGSFQDLDLSSMGNGGIQLQFSHQLYNKTFKHVVSIIVAMEKLKKIPVPCSQAFQDDDLRSLFSVIFEEEPIICDNWDDDYVCDAAVHSVNCRLRDIYHKSLVLSGACELQAVHLNGENTNQQVVFCMSFVQGEEETDKIPVALGLKEKNLYLSCGMKDGKPTLQLETVDPNTYPKRKMEKRFVFNKMEIKGNVEFESAMYPNWYISTSQAEKKPVFLGNTRGGRDITDFIMEITSA

SEQ ID NO: 56 - protein, 267 aa, Felis catus
MAPVPELTSEMMAYYSDENDLFFEADGPEKMKGSLQNLSHSFLGDEGIQLQISHQPDNKSLRHAVSVIVAMEKLKKISFACSQPLQDEDLKSLFCCIFEEEPIICDTWDDGFVCDAAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESAS

SEQ ID NO: 57 - protein, 267 aa, Sus scrofa
MATVPEPAKEVMANNGDNNNDLLFEADGPKEMKCRTQNLDLSPLGDGSIQLQISHQLCNESSRPMVSVIVAKEEPMNPSSQVVCDDDPKSIFSSVFEEEPIVLEKHANGFLCDATPVQSVDCKLQDKDEKALVLAGPHELKALHLLKGDLKREVVFCMSFVQGDDSDDKIPVTLGIKGKNLYLSCVMKDDTPTLQLEDVDPKSYPKRDMEKRFVFYKTEIKNRVEFESALYPNWYISTSQAEQKPVFLGNSKGRQDITDFTMEVLSP

SEQ ID NO: 58 - protein, 266 aa, Cavia porcellus
MAAVPELSSEVTAYHSDENELFFEVDGPNKMQYCFQDRDLCSLDEGIKLQISHQHFNKSFRQTVSLIVAVEKLRKKLAPCTWAFQDDDLRPLLPFIFEEEPIVCDTWDEEYESDTPVPSRNCTLHDIQHKRLVLSDPCELKALHLNGDNLNRQVVFSMSFVQGERSDNKMPVALGLKGKNLYLSCVMKDGKPVLQLESVDGKQYPKKKMEKRFVFNKITSKSTVEFESAQFPNWYISTSQAEHKPVFLGNNNGQDIIDFKLELVSS

SEQ ID NO: 59 - protein, 266 aa, Bos taurus
MATVPEPINEMMAYYSDENELLFEADDPKQMKSCIQHLDLGSMGDGNIQLQISHQFYNKSFRQVVSVIVAMEKLRNSAYAHVFHDDDLRSILSFIFEEEPVIFETSSDEFLCDAPVQSIKCKLQDREQKSLVLASPCVLKALHLLSQEMNREVVFCMSFVQGEERDNKIPVALGIKDKNLYLSCVKKGDTPTLQLEEVDPKVYPKRNMEKRFVFYKTEIKNTVEFESVLYPNWYISTSQIEERPVFLGHFRGGQDITDFRMETLSP

SEQ ID NO: 60 - protein, 266 aa, Ovis aries
MATVPEPINEVMAYYSDENELLFEVDGPKQMKSCTQHLDLGSMGDGNIQLQISHQLYNKSFRQVVSVIVAMEKLRSRAYEHVFRDDDLRSILSFIFEEEPVIFETSSDELLCDAAVQSVKCKLQDREQKSLVLDSPCVLKALHLPSQEMSREVVFCMSFVQGEERDNKIPVALGIRDKNLYLSCVKKGDTPTLQLEEVDPKVYPKRNMEKRFVFYKTEIKNTVEFESVLYPNWYISTSQIEEKPVFLGRFRGGQDITDFRMETLSP

SEQ ID NO: 61 - protein, 267 aa, Gallus gallus
MAFVPDLDVLESSSLSEETFYGPSCLCLQKKPRLDSEHTTVDVQVTVRKGRGARSFRRAAVLVVAMTKLLRRPRSRDFADSDLSALLEEVFEPVTFQRLESSYAGAPAFRYTRSQSFDIFDINQKCFVLESPTQLVALHLQGPSSSQKVRLNIALYRPRGPRGSAGTGQMPVALGIKGYKLYMSCVMSGTEPTLQLEEADVMRDIDSVELTRFIFYRLDSPTEGTTRFESAAFPGWFICTSLQPRQPVGITNQPDQVNIATYKLSGR

SEQ ID NO: 62 - protein, 127 aa, Canis familiaris
GPGGSNVKCCCQDLNHSSLVDEGIQLQVSHQLCNKSLRHFVSVIVALEKLKKPCPQVLQEDDLKSIFCYIFEEEPIICKTDADNFMSDAAMQSVDCKLQDISHKYLVLSNSYELRALHLNGENVNKA

SEQ ID NO: 63 - protein, 153 aa, Homo sapiens
LSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 64 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 65 - protein, 183 aa, Mus musculus
MDPHHHHHHGSGDDDDKALAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQISLEGGGGGCG

SEQ ID NO: 66 - protein, 180 aa, Mus musculus
MDPHHHHHHGSGDDDDKALAPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSSLEGGGGGCG

SEQ ID NO: 67 - protein, 12 aa, Homo sapiens
MRIIKYEFILND

SEQ ID NO: 68 - protein, 6 aa, Homo sapiens
RIIKYE

SEQ ID NO: 69 - protein, 10 aa, Homo sapiens
YEFILNDALN

SEQ ID NO: 70 - protein, 12 aa, Homo sapiens
NDALNQSIIRAN

SEQ ID NO: 71 - protein, 10 aa, Homo sapiens
TAAALHNLDE

SEQ ID NO: 72 - protein, 12 aa, Homo sapiens
TAAALHNLDEAV

SEQ ID NO: 73 - protein, 10 aa, Homo sapiens
AVKFDMGAYK

SEQ ID NO: 74 - protein, 11 aa, Homo sapiens
AYKSSKDDAKI

SEQ ID NO: 75 - protein, 31 aa, Homo sapiens
SSKDDAKITVILRISKTQLYVTAQDEDQPVL

SEQ ID NO: 76 - protein, 13 aa, Homo sapiens
VILRISKTQLYVT

SEQ ID NO: 77 - protein, 22 aa, Homo sapiens
VILRISKTQLYVTAQDEDQPVL

SEQ ID NO: 78 - protein, 20 aa, Homo sapiens
TQLYVTAQDEDQPVLLKEMP

SEQ ID NO: 79 - protein, 21 aa, Homo sapiens
PVLLKEMPEIPKTITGSETNL

SEQ ID NO: 80 - protein, 14 aa, Homo sapiens
EMPEIPKTITGSET

SEQ ID NO: 81 - protein, 12 aa, Homo sapiens
KTITGSETNLLF

SEQ ID NO: 82 - protein, 21 aa, Homo sapiens
PKTITGSETNLLFFWETHGTK

SEQ ID NO: 83 - protein, 10 aa, Homo sapiens
GSETNLLFFW

SEQ ID NO: 84 - protein, 12 aa, Homo sapiens
LFIATKQDYWVC

SEQ ID NO: 85 - protein, 13 aa, Homo sapiens
FIATKQDYWVCLA

SEQ ID NO: 86 - protein, 18 aa, Homo sapiens
IATKQDYWVCLAGGPPSI

SEQ ID NO: 87 - protein, 5 aa, Homo sapiens
KQDYW

SEQ ID NO: 88 - protein, 7 aa, Homo sapiens
QILENQA

SEQ ID NO: 89 - protein, 6 aa, Homo sapiens
APVRSL

SEQ ID NO: 90 - protein, 12 aa, Homo sapiens
APVRSLNCTLRD

SEQ ID NO: 91 - protein, 10 aa, Homo sapiens
LNCTLRDSQQ

SEQ ID NO: 92 - protein, 12 aa, Homo sapiens
RDSQQKSLVMSG

SEQ ID NO: 93 - protein, 12 aa, Homo sapiens
KALHLQGQDMEQ

SEQ ID NO: 94 - protein, 14 aa, Homo sapiens
KALHLQGQDMEQQV

SEQ ID NO: 95 - protein, 10 aa, Homo sapiens
QVVFSMSFVQ

SEQ ID NO: 96 - protein, 11 aa, Homo sapiens
FVQGEESNDKI

SEQ ID NO: 97 - protein, 32 aa, Homo sapiens
GEESNDKIPVALGLKEKNLYLSCVLKDDKPTL

SEQ ID NO: 98 - protein, 13 aa, Homo sapiens
VALGLKEKNLYLS

SEQ ID NO: 99 - protein, 23 aa, Homo sapiens
VALGLKEKNLYLSCVLKDDKPTL

SEQ ID NO: 100 - protein, 21 aa, Homo sapiens
KNLYLSCVLKDDKPTLQLESV

SEQ ID NO: 101 - protein, 20 aa, Homo sapiens
PTLQLESVDPKNYPKKKMEK

SEQ ID NO: 102 - protein, 13 aa, Homo sapiens
ESVDPKNYPKKKM

SEQ ID NO: 103 - protein, 12 aa, Homo sapiens
KNYPKKKMEKRF

SEQ ID NO: 104 - protein, 10 aa, Homo sapiens
KKKMEKRFVF

SEQ ID NO: 105 - protein, 18 aa, Homo sapiens
KKKMEKRFVFNKIEINNK

SEQ ID NO: 106 - protein, 14 aa, Homo sapiens
WYISTSQAENMPVF

SEQ ID NO: 107 - protein, 15 aa, Homo sapiens
YISTSQAENMPVFLG

SEQ ID NO: 108 - protein, 22 aa, Homo sapiens
ISTSQAENMPVFLGGTKGGQDI

SEQ ID NO: 109 - protein, 6 aa, Homo sapiens
QAENMP

SEQ ID NO: 110 - protein, 7 aa, Homo sapiens
TMQFVSS

SEQ ID NO: 111 - protein, 12 aa, Mus musculus
VPIRQLHYRLRD

SEQ ID NO: 112 - protein, 14 aa, Mus musculus
KALHLNGQNINQQV

SEQ ID NO: 113 - protein, 15 aa, Mus musculus
YISTSQAEHKPVFLG

SEQ ID NO: 114 - protein, 20 aa, Rattus norvegicus
NLYLSCVMKDGTPTLQLESV

SEQ ID NO: 115 - protein, 20 aa, Rattus norvegicus
PTLQLESVDPKQYPKKKMEK

SEQ ID NO: 116 - protein, 22 aa, Rattus norvegicus
ISTSQAEHRPVFLGNSNGRDIV

SEQ ID NO: 117 - DNA, 421 bp, artificial sequence
  synthesized sequence
atggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttagggatccggataatgcatctaagctt

SEQ ID NO: 118 - DNA, 421 bp, artificial sequence
  synthesized sequence
atggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttgaggatccggataatgcatctaagctt

SEQ ID NO: 119 - DNA, 28 bp, artificial sequence
  synthesized sequence
nnccatggcaaataagccaatgcaaccg

SEQ ID NO: 120 - DNA, 52 bp, artificial sequence
  synthesized sequence
gtaagcttagatgcattatccggatccctaagcagtagtatcagacgatacg

SEQ ID NO: 121 - DNA, 52 bp, artificial sequence
  synthesized sequence
gtaagcttagatgcattatccggatcctcaagcagtagtatcagacgatacg

SEQ ID NO: 122 - DNA, 25 bp, artificial sequence
  synthesized sequence
ggtccggagcgctagccccttacac

SEQ ID NO: 123 - DNA, 40 bp, artificial sequence
  synthesized sequence
gtaagcttatgcattatgatatctggaagtctgtcataga

SEQ ID NO: 124 - DNA, 36 bp, artificial sequence
  synthesized sequence
atatatgatatccctgtacgatcactgaactgcacg

SEQ ID NO: 125 - DNA, 37 bp, artificial sequence
  synthesized sequence
atatatctcgagggaagacacaaattgcatggtgaag

SEQ ID NO: 126 - DNA, 48 bp, artificial sequence
  synthesized sequence
tatggatatcgaattcaagcttctgcagctgctcgagtaattgattac

SEQ ID NO: 127 - DNA, 50 bp, artificial sequence
  synthesized sequence
ctaggtaatcaattactcgagcagctgcagaagcttgaattcgatatcca

SEQ ID NO: 128 - DNA, 52 bp, artificial sequence
  synthesized sequence
tcgagcaccaccaccaccaccacggtggttgctaataataattgattaatac

SEQ ID NO: 129 - DNA, 52 bp, artificial sequence
  synthesized sequence
ctaggtattaatcaattattattagcaaccaccgtggtggtggtggtggtgc

SEQ ID NO: 130 - protein, 166 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSSLEHHHHHHGGC

SEQ ID NO: 131 - protein, 155 aa, Homo sapiens
MDIPVDSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 132 - protein, 155 aa, Homo sapiens
MDIPVRSANCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 133 - protein, 155 aa, Homo sapiens
MDIPVRSLNCGLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 134 - protein, 155 aa, Homo sapiens
MDIPVRSLNCTLGDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 135 - protein, 155 aa, Homo sapiens
MDIPVRSLNCTLRDSQQKSLVMSGPYELKALHLQGQDMEQQVVFSMSFVQGEESNRKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 136 - protein, 155 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSS

SEQ ID NO: 137 - protein, 153 aa, Homo sapiens
MDIPVRSLNCTLRDSQQKSLVMSGPYELKALHLQGQDMEQQVVFSMSFVQGSNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 138 - protein, 152 aa, Homo sapiens
MDIPVRSLNCTLRDSQQKSLVMSGPYELKALHLQGQDMEQQVVFSMSFVQGEEKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 139 - protein, 155 aa, Homo sapiens
MDIPVRSLNCTLRDSQQKSLVMSGPYELKALHLQGQDMEQQVVFSMSFVQGEESNDKIPVALGLSESNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 140 - protein, 155 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSAAANMPVFLGGTKGGQDITDFTMQFVSS

SEQ ID NO: 141 - DNA, 39 bp, artificial sequence
  synthesized sequence
atatatcatatgctgagcaatgtgaaatacaactttatg

SEQ ID NO: 142 - DNA, 37 bp, artificial sequence
  synthesized sequence
atatatctcgagcgcctggttttccagtatctgaaag

SEQ ID NO: 143 - DNA, 39 bp, artificial sequence
  synthesized sequence
catatggatatccctgtagactcactgaactgcacgctc

SEQ ID NO: 144 - DNA, 39 bp, artificial sequence
  synthesized sequence
gagcgtgcagttcagtgagtctacagggatatccatatg

SEQ ID NO: 145 - DNA, 39 bp, artificial sequence
  synthesized sequence
gatatccctgtacgatcagctaactgcacgctccgggac

SEQ ID NO: 146 - DNA, 39 bp, artificial sequence
  synthesized sequence
gtcccggagcgtgcagttagctgatcgtacagggatatc

SEQ ID NO: 147 - DNA, 37 bp, artificial sequence
  synthesized sequence
gtacgatcactgaactgcggtctccgggactcacagc

SEQ ID NO: 148 - DNA, 37 bp, artificial sequence
  synthesized sequence
gctgtgagtcccggagaccgcagttcagtgatcgtac

SEQ ID NO: 149 - DNA, 26 bp, artificial sequence
  synthesized sequence
gaactgcacgctcggggactcacagc

SEQ ID NO: 150 - DNA, 26 bp, artificial sequence
  synthesized sequence
gctgtgagtccccgagcgtgcagttc

SEQ ID NO: 151 - DNA, 39 bp, artificial sequence
  synthesized sequence
caaggagaagaaagtaatcgcaaaatacctgtggccttg

SEQ ID NO: 152 - DNA, 39 bp, artificial sequence
  synthesized sequence
caaggccacaggtattttgcgattactttcttctccttg

SEQ ID NO: 153 - DNA, 40 bp, artificial sequence
  synthesized sequence
catgtcctttgtacaaggaagtaatgacaaaatacctgtg

SEQ ID NO: 154 - DNA, 40 bp, artificial sequence
  synthesized sequence
cacaggtattttgtcattacttccttgtacaaaggacatg

SEQ ID NO: 155 - DNA, 37 bp, artificial sequence
  synthesized sequence
ctttgtacaaggagaagaaaaaatacctgtggccttg

SEQ ID NO: 156 - DNA, 37 bp, artificial sequence
  synthesized sequence
caaggccacaggtattttttcttctccttgtacaaag

SEQ ID NO: 157 - DNA, 41 bp, artificial sequence
  synthesized sequence
gtggccttgggcctcagcgaaagcaatctgtacctgtcctg

SEQ ID NO: 158 - DNA, 41 bp, artificial sequence
  synthesized sequence
caggacaggtacagattgctttcgctgaggcccaaggccac

SEQ ID NO: 159 - DNA, 40 bp, artificial sequence
  synthesized sequence
gtacatcagcacctctgcagcagcaaacatgcccgtcttc

SEQ ID NO: 160 - DNA, 40 bp, artificial sequence
  synthesized sequence
gaagacgggcatgtttgctgctgcagaggtgctgatgtac

SEQ ID NO: 161 - DNA, 40 bp, artificial sequence
  synthesitzed sequence
gcggccaggatataactaaattcaccatgcaatttgtgtc

SEQ ID NO: 162 - DNA, 40 bp, artificial sequence
  synthesized sequence
gacacaaattgcatggtgaatttagttatatcctggccgc

SEQ ID NO: 163 - protein, 154 aa, Mus musculus
PYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 164 - protein, 151 aa, Mus musculus
PIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 165 - protein, 155 aa, Mus musculus
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 166 - protein, 569 aa, Homo sapiens [sequence omitted]

SEQ ID NO: 167 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 168 - protein, 576 aa, Mus musculus
MENMKVLLGLICLMVPLLSLEIDVCTEYPNQIVLFLSVNEIDIRKCPLTPNKMHGDTIIWYKNDSKTPISADRDSRIHQQNEHLWFVPAKVEDSGYYYCIVRNSTYCLKTKVTVTVLENDPGLCYSTQATFPQRLHIAGDGSLVCPYVSYFKDENNELPEVQWYKNCKPLLLDNVSFFGVKDKLLVRNVAEEHRGDYICRMSYTFRGKQYPVTRVIQFITIDENKRDRPVILSPRNETIEADPGSMIQLICNVTGQFSDLVYWKWNGSEIEWNDPFLAEDYQFVEHPSTKRKYTLITTLNISEVKSQFYRYPFICVVKNTNIFESAHVQLIYPVPDFKNYLIGGFIILTATIVCCVCIYKVFKVDIVLWYRDSCSGFLPSKASDGKTYDAYILYPKTLGEGSFSDLDTFVFKLLPEVLEGQFGYKLFIYGRDDYVGEDTIEVTNENVKKSRRLIIILVRDMGGFSWLGQSSEEQIAIYNALIQEGIKIVLLELEKIQDYEKMPDSIQFIKQKHGVICWSGDFQERPQSAKTRFWKNLRYQMPAQRRSPLSKHRLLTLDPVRDTKEKLPAATHLPLG

SEQ ID NO: 169 - protein, 410 aa, Mus musculus
MFILLVLVTGVSAFTTPTVVHTGKVSESPITSEKPTVHGDNCQFRGREFKSELRLEGEPVVLRCPLAPHSDISSSSHSFLTWSKLDSSQLIPRDEPRMWVKGNILWILPAVQQDSGTYICTFRNASHCEQMSVELKVFKNTEASLPHVSYLQISALSTTGLLVCPDLKEFISSNADGKIQWYKGAILLDKGNKEFLSAGDPTRLLISNTSMDDAGYYRCVMTFTYNGQEYNITRNIELRVKGTTTEPIPVIISPLETIPASLGSRLIVPCKVFLGTGTSSNTIVWWLANSTFISAAYPRGRVTEGLHHQYSENDENYVEVSLIFDPVTREDLHTDFKCVASNPRSSQSLHTTVKEVSSTFSWSIALAPLSLIILVVGAIWMRRRCKRRAGKTYGLTKLRTDNQDFPSSPN

SEQ ID NO: 170 - protein, 5 aa, artificial sequence
  synthesized sequence
GGKGG

SEQ ID NO: 171 - protein, 3 aa, artificial sequence
  synthesized sequence
CGG

SEQ ID NO: 172 - protein, 10 aa, artificial sequence
  synthesized sequence
CGDKTHTSPP

SEQ ID NO: 173 - protein, 17 aa, artificial sequence
  synthesized sequence
CGGPKPSTPPGSSGGAP

SEQ ID NO: 174 - protein, 6 aa, artificial sequence
  synthesized sequence
GCGGGG

SEQ ID NO: 175 - protein, 18 aa, artificial sequence
  synthesized sequence
GGGGGCGGGGGGGGGGGG

SEQ ID NO: 176 - protein, 15 aa, artificial sequence
  synthesized sequence
GGGGSGGGGSGGGGS

SEQ ID NO: 177 - protein, 33 aa, artificial sequence
  synthesized sequence
GGGGGCGGGGGGGGGGSSGGGGSGGGGSGGGGS

SEQ ID NO: 178 - protein, 3 aa, artificial sequence
  synthesized sequence
GGC

SEQ ID NO: 179 - protein, 3 aa, artificial sequence
  synthesized sequence
GGC

SEQ ID NO: 180 - protein, 10 aa, artificial sequence
  synthesized sequence
DKTHTSPPCG

SEQ ID NO: 181 - protein, 18 aa, artificial sequence
  synthesized sequence
PKPSTPPGSSGGAPGGCG

SEQ ID NO: 182 - protein, 6 aa, artificial sequence
  synthesized sequence
GGGGCG

SEQ ID NO: 183 - protein, 18 aa, artificial sequence
  synthesized sequence
GGGGGGGGGGGGCGGGGG

SEQ ID NO: 184 - protein, 15 aa, artificial sequence
  synthesized sequence
SGGGGSGGGGSGGGG

SEQ ID NO: 185 - protein, 41 aa, artificial sequence
  synthesized sequence
GGGGGGGGGGSSGGGGSGGGGSGGGGSGGGGGGGGCGGGGG

SEQ ID NO: 186 - protein, 6 aa, artificial sequence
  synthesized sequence
GGKKGC

SEQ ID NO: 187 - protein, 6 aa, artificial sequence
  synthesized sequence
CGKKGG

SEQ ID NO: 188 - protein, 4 aa, artificial sequence
  synthesized sequence
GGCG

SEQ ID NO: 189 - protein, 3 aa, artificial sequence
  synthesized sequence
GSG

SEQ ID NO: 190 - protein, 11 aa, artificial sequence
  synthesized sequence
LEHHHHHHGGC

SEQ ID NO: 191 - protein, 21 aa, artificial sequence
  synthesized sequence
MEVGWYRSPFSRVVHLYRNGK

SEQ ID NO: 192 - DNA, 31 bp, artificial sequence
  synthesized sequence
gatccggaggtggtgtccccattagacagct

SEQ ID NO: 193 - DNA, 27 bp, artificial sequence
  synthesized sequence
gtaagcttaggaagacacagattccat

SEQ ID NO: 194 - DNA, 37 bp, artificial sequence
  synthesized sequence
gatccggaggtggtgcccctgtacgatcactgaactg

SEQ ID NO: 195 - DNA, 37 bp, artificial sequence
  synthesized sequence
gtatgcattaggaagacacaaattgcatggtgaagtc

SEQ ID NO: 196 - DNA, 37 bp, artificial sequence
  chemically synthesized
atatatcatatgtctgccccttacacctaccagagtg

SEQ ID NO: 197 - DNA, 39 bp, artificial sequence
  chemically synthesized
ggactgccctctatgacaaaattccagatatcactcgag

SEQ ID NO: 198 - DNA, 39 bp, artificial sequence
  chemically synthesized
ctcgagtgatatctggaattttgtcatagagggcagtcc

SEQ ID NO: 199 - DNA, 40 bp, artificial sequence
  chemically synthesized
gggccaccctctatcactaaatttcagatactggaaaacc

SEQ ID NO: 200 - DNA, 40 bp, artificial sequence
  chemically synthesized
ggttttccagtatctgaaatttagtgatagagggtggccc

SEQ ID NO: 201 - protein, 11 aa, artificial sequence
  chemically synthesized
LEHHHHHHGGC

SEQ ID NO: 202 - protein, 157 aa, Mus musculus
MSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 203 - protein, 154 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 204 - protein, 157 aa, Mus musculus
MSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTKFQIS

SEQ ID NO: 205 - protein, 155 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPNNYPKKKMEKRFVFNKIEINNKLEFESAQFPNWYISTSQAENMPVFLGGTKGGQDITDFTMQFVSS

SEQ ID NO: 206 - protein, 155 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKQFVFNKIEINNKLEFESAQFPNWYISTSQAENMPVFLGGTKGGQDITDFTMQFVSS

SEQ ID NO: 207 - protein, 155 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNLIEINNKLEFESAQFPNWYISTSQAENMPVFLGGTKGGQDITDFTMQFVSS

SEQ ID NO: 208 - protein, 152 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPMEKRFVFNKIEINNKLEFESAQFPNWYISTSQAENMPVFLGGTKGGQDITDFTMQFVSS

SEQ ID NO: 209 - protein, 155 aa, Homo sapiens
MDIPVRSLNCTNRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 210 - protein, 154 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITKFQILENQA

SEQ ID NO: 211 - protein, 154 aa, Homo sapiens
MLSNVKYNFMRIIKYEFIKNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 212 - protein, 154 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDNQILENQA

SEQ ID NO: 213 - protein, 154 aa, Homo sapiens
MLSNVKYNFMAIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 214 - protein, 154 aa, Homo sapiens
MLSNVKYNFMRIIKYEFILNDALNQSIIRANDQYLTAAALHNLDEAVKFDMGAYKSSKDDAKATVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 215 - protein, 154 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFFETHGTKNYFTSVAHPNLFIATKQDYWVCLAGGPPSITDFQILENQA

SEQ ID NO: 216 - protein, 154 aa, Homo sapiens
MLSNVKYNFMRIIKYEFILNVALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 217 - protein, 151 aa, Homo sapiens
MLSNVKYNFMRIIKYENDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 218 - protein, 150 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTETNLLFFWETHGTKNYFTSVAHPNLFIATKQDYWVCLAGGPPSITDFQILENQA

SEQ ID NO: 219 - protein, 12 aa, artificial sequence
  His Tag
LEHHHHHHGGCG

SEQ ID NO: 220 - protein, 167 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSLEHHHHHHGGCG

SEQ ID NO: 221 - protein, 159 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSGGCG

SEQ ID NO: 222 - protein, 158 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSGGC

SEQ ID NO: 223 - protein, 159 aa, Homo sapiens
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSSGGCG

SEQ ID NO: 224 - protein, 158 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQAGGCG

SEQ ID NO: 225 - protein, 158 aa, Homo sapiens
MLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITKFQILENQAGGCG

SEQ ID NO: 226 - DNA, 36 bp, artificial sequencce
  chemically synthesized
atatatgatatccccattagacagctgcactacagg

SEQ ID NO: 227 - DNA, 37 bp, artificial sequence; identical to SEQ ID NO: 27
  chemically synthesized

SEQ ID NO: 228 - protein, 165 aa, Mus musculus
MDIPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIKFTMESVSSLEHHHHHHGGC

SEQ ID NO: 229 - DNA, 42 bp, artificial sequence
  chemically synthesized
cagtggtcaggacataattaaattcaccatggaatctgtgtc

SEQ ID NO: 230 - DNA, 42 bp, artificial sequence
  chemically synthesized
gacacagattccatggtgaatttaattatgtcctgaccactg

(19) United States
(12) Patent Application Publication
(10) Pub. No.: US 2011/0318300 A1
(43) Pub. Date: Dec. 29, 2011

(54) User of Interleukin-1 Conjugates in the Treatment of Diabetes

(75) Inventors: Martin Bachmann, Seuzach (CH); Gunther Spohn, Zurich (CH)

(73) Assignee: Cytos Biotechnology AG

(21) Appl. No.: 12/921,038

(22) PCT Filed: Mar. 5, 2009
(86) PCT No.: PCT/EP09/52639

§ 371 Date: May 2, 2011

(30) Foreign Application Priority Data
Mar. 5, 2008 (EP) 08152336.7

(51) Int. Cl. A61K 38/20; A61P 3/10

(52) U.S. Cl. 424/85.2

(57) ABSTRACT

The invention provides compositions, pharmaceutical compositions and vaccines for the treatment, amelioration and/or prophylaxis of diabetes, preferably of type II diabetes. The compositions, pharmaceutical compositions and vaccines of the invention comprise a core particle and an antigen, wherein said antigen comprises an interleukin-1 (IL-1) molecule. When administered to an animal, preferably to a human, said compositions, pharmaceutical compositions, and vaccines induce efficient immune responses, in particular antibody responses, wherein typically and preferably said antibody responses are directed against IL-1. Thus, the invention provides methods of treating, ameliorating or preventing diabetes, preferably type II diabetes, by way of active immunization against IL-1.

### FIELD OF THE INVENTION

The present invention is in the fields of medicine, public health, immunology, molecular biology and virology. The invention provides compositions, pharmaceutical compositions and vaccines for the treatment, amelioration and/or prophylaxis of diabetes, preferably of type II diabetes. The compositions, pharmaceutical compositions and vaccines of the invention comprise a core particle and an antigen, wherein said antigen comprises an interleukin-1 (IL-1) molecule. When administered to an animal, preferably to a human, said compositions, pharmaceutical compositions, and vaccines induce efficient immune responses, in particular antibody responses, wherein typically and preferably said antibody responses are directed against IL-1. Thus, the invention provides methods of treating, ameliorating or preventing diabetes, preferably type II diabetes, by way of active immunization against IL-1.

### RELATED ART

Type 2 diabetes is a chronic metabolic disorder characterized by the presence of hyperglycemia due to defective insulin secretion, insulin action or a combination of both. Although the mechanisms of pancreatic β-cell failure in type 2 diabetes are not fully elucidated, stress and inflammatory pathways have been implicated. Metabolic stress caused by repetitive glucose excursions, dyslipidemia and adipokines can induce an inflammatory response in the pancreas characterized by local cytokine secretion, islet immune-cell infiltration, β-cell apoptosis, amyloid deposits and fibrosis. IL-1β has emerged as a master cytokine, which regulates islet chemokine production and causes impaired insulin production and β-cell death. Blockade of IL-1 signalling by administration of recombinant IL-1 receptor antagonist or neutralizing monoclonal antibodies has been shown to improve glycemic control in animal models of type 2 diabetes (Sauter et al., 2008, Osborn et al., 2008). Furthermore, treatment of type 2 diabetes patients with recombinant human IL-1 receptor antagonist (Anakinra) resulted in a decrease of glycated haemoglobin levels (a reliable readout for long term glycemia) and improved β-cell function (Larsen et al., 2007).

### SUMMARY OF THE INVENTION

We have found that the inventive compositions and vaccines, respectively, comprising at least one IL-1 molecule, preferably a IL-1 mutein, are not only capable of inducing immune responses against IL-1, and hereby in particular antibody responses, but are, furthermore, capable of neutralizing the pro-inflammatory activity of IL-1 in vivo. In addition, we have surprisingly found active immunization with a composition of the invention resulted in the amelioration of the diet-induced diabetic phenotype in a mouse model (cf. Surwit et al., DIABETES, Vol. 37, 1988, 1163-1167) of diabetes. This observation was made for different IL-1 molecules including IL-1 alpha molecules (cf. Example 9) as well as IL-1 beta molecules (cf. Examples 12 and 13).

In one aspect, the invention therefore provides a composition for the treatment, amelioration or prophylaxis of diabetes, preferably of type II diabetes, wherein said composition comprises: (a) a core particle with at least one first attachment site, wherein said core particle is a virus-like particle (VLP) or a virus particle, preferably a virus-like particle; and (b) at least one antigen with at least one second attachment site, wherein the at least one antigen comprises or consists of or is an IL-1 molecule, preferably selected from the group consisting of IL-1 protein, IL-1 mature fragment, IL-1 peptide and IL-1 mutein, wherein (a) and (b) are linked, preferably covalently linked, through the at least one first and the at least one second attachment site.

In a further aspect, the invention provides a composition comprising (a) a virus-like particle (VLP) with at least one first attachment site; and (b) at least one antigen with at least one second attachment site; wherein said at least one antigen comprises consists of or is an IL-1 molecule and wherein (a) and (b) are linked through said at least one first and said at least one second attachment site. In a preferred embodiment said at least one antigen with at least one second attachment site comprises or preferably consists of (i) an IL-1 molecule; and (ii) a linker.

In a further preferred embodiment wherein said first attachment site is linked to said second attachment site via at least one covalent bond, wherein preferably said at least one covalent bond is a non-peptide bond.

In a further preferred embodiment said at least one antigen with at least one second attachment site comprises or preferably consists of: (i) an IL-1 beta molecule, wherein said IL-1 beta molecule is SEQ ID NO:165 or SEQ ID NO:136, preferably SEQ ID NO:136; and (ii) a linker, wherein said linker comprises said second attachment site, and wherein preferably said linker comprises or preferably consists of GGC (SEQ ID NO:178) or GGCG (SEQ ID NO:188), preferably GGCG (SEQ ID NO:188); wherein further preferably said linker is covalently bound to the C-terminus of said IL-1 beta molecule by way of a peptide bond.

In a further preferred embodiment said at least one antigen with at least one second attachment site consists of (i) an IL-1 alpha molecule, wherein said IL-1 alpha molecule is SEQ ID NO:203 or SEQ ID NO:210, preferably SEQ ID NO:203; and (ii) a linker, wherein said linker comprises said second attachment site, and wherein preferably said linker comprises or preferably consists of GGC (SEQ ID NO:178) or GGCG (SEQ ID NO:188), preferably GGCG (SEQ ID NO:188); wherein further preferably said linker is covalently bound to the C-terminus of said IL-1 alpha molecule by way of a peptide bond.

In a further preferred embodiment said virus-like particle is a virus-like particle of an RNA bacteriophage, wherein preferably said RNA bacteriophage is bacteriophage Qβ.

In a further preferred embodiment only one of said second attachment sites associates with said first attachment site through at least one non-peptide covalent bond leading to a single and uniform type of binding of said antigen to said virus-like particle, wherein said only one second attachment site that associates with said first attachment site is a sulfhydryl group, and wherein said antigen and said virus-like particle interact through said association to form an ordered and repetitive antigen array.

In a further aspect, the invention provides a vaccine for the treatment of diabetes, preferably of type II diabetes, said vaccine comprising, or alternatively consisting of the composition of the invention, preferably in an effective amount.

In a further aspect, the invention provides a pharmaceutical composition for the treatment of diabetes, preferably of type II diabetes, said pharmaceutical composition comprising: (a) the composition of the invention or the vaccine of the invention; and (b) a pharmaceutically acceptable carrier.

In a further aspect, the invention provides a method of treating diabetes, preferably type II diabetes, said method comprising administering an immunologically effective amount of the composition of the invention, of the vaccine of the invention, and/or of the pharmaceutical composition of the invention to an animal, preferably to a human.

In a further aspect, the invention provides the use of the composition of the invention, of the vaccine of the invention, and/or of the pharmaceutical composition of the invention for the manufacture of a medicament for treatment of diabetes, preferably of type II diabetes, in an animal, preferably in a human.

### DETAILED DESCRIPTION OF THE INVENTION

Unless defined otherwise, all technical and scientific terms used herein have the same meanings as commonly understood by one of ordinary skill in the art to which this invention belongs.

Adjuvant: The term “adjuvant” as used herein refers to non-specific stimulators of the immune response or substances that allow generation of a depot in the host which when combined with the vaccine or with the pharmaceutical composition, respectively, may provide for an even more enhanced immune response. Preferred adjuvants are complete and incomplete Freund's adjuvant, aluminum containing adjuvant, preferably aluminum hydroxide, most preferably alum, and modified muramyldipeptide. Further preferred adjuvants are mineral gels such as aluminum hydroxide, surface active substances such as lysolecithin, pluronic polyols, polyanions, peptides, oil emulsions, keyhole limpet hemocyanins, dinitrophenol, and human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants can also comprise a mixture of these substances. VLP have been generally described as an adjuvant. However, the term “adjuvant”, as used within the context of this application, refers to an adjuvant not being the VLP used for the inventive compositions, rather it relates to an additional, distinct component.

Antigen: As used herein, the term “antigen” refers to a molecule capable of being bound by an antibody or a T-cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. The term “antigen” as used herein preferably refers to the IL-1 molecule, the IL-1 protein, IL-1 mature fragment, the IL-1 fragment, the IL-1 peptide and the IL-1 mutein, most preferably “antigen” refers to the IL-1 mutein. If not indicated otherwise, the term “antigen” as used herein does not refer to the virus particle or to the virus-like particle.

Epitope: “epitope” refers to continuous or discontinuous portions of a polypeptide which can be bound immunospecifically by an antibody or by a T-cell receptor within the context of an MHC molecule. Immunospecific binding excludes non-specific binding but does not necessarily exclude cross-reactivity. An epitope typically comprise 5-10 amino acids in a spatial conformation which is unique to the epitope.

Specific binding (antibody/antigen): Within this application, antibodies are defined to be specifically binding if they bind to the antigen with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The affinity of an antibody can be readily determined by one of ordinary skill in the art (for example by Scatchard analysis, by ELISA or by Biacore analysis).

Specific binding (IL-1/IL-1 receptor): The interaction between a receptor and a receptor ligand can be characterized by biophysical methods generally known in the art, including, for example, ELISA or Biacore analysis. An IL-1 molecule is regarded as capable of specifically binding an IL-1 receptor, when the binding affinity (Ka) of said IL-1 to said IL-1 receptor is at least 10⁵ M⁻¹, preferably at least 10⁶ M⁻¹, more preferably at least 10⁷ M⁻¹, still more preferably at least 10⁸ M⁻¹, and most preferably at least 10⁹ M′; wherein preferably said IL-1 receptor is an IL-1 receptor from mouse or human, most preferably human. Further preferably, said IL-1 receptor comprises or more preferably consists of any one of the sequences SEQ ID NO:166 to SEQ ID NO:169, most preferably said IL-1 receptor comprises or preferably consists of any one of the sequences SEQ ID NO:166 and SEQ ID NO:167.

Associated: The terms “associated” or “association” as used herein refer to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds.

Attachment Site, First: As used herein, the phrase “first attachment site” refers to an element which is naturally occurring with the VLP, preferably with the VLP of an RNA bacteriophage, or which is artificially added to the VLP, preferably to the VLP of an RNA bacteriophage, and to which the second attachment site may be linked. The first attachment site preferably is a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid, preferably of lysine. In a preferred embodiment said first attachment site is the amino group of a lysine residue, wherein preferably said lysine residue is a lysine residue which is naturally occurring with said VLP, preferably with said VLP of an RNA bacteriophage. The first attachment site is located, typically on the surface, and preferably on the outer surface of the VLP, preferably of the VLP of an RNA bacteriophage, most preferably of RNA bacteriophage Qβ. Multiple first attachment sites are present on the surface, preferably on the outer surface of the virus-like particle, preferably of the VLP of an RNA bacteriophage, most preferably of the VLP of RNA bacteriophage Qβ, typically and preferably in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in a preferred embodiment the first attachment site is artificially added to the VLP. In a preferred embodiment the first attachment site is associated with said VLP through at least one covalent bond, preferably through at least one peptide bond, wherein said VLP is a VPL of an RNA bacteriophage, preferably of RNA bacteriophage Qβ. In a further preferred embodiment said first attachment site is the amino group of a lysine residue, wherein said lysine residue is a lysine residue of a coat protein, preferably of a coat protein of an RNA bacteriophage, most preferably of RNA bacteriophage Qβ. In a further preferred embodiment said first attachment site is an amino group of a lysine residue of a coat protein of an RNA bacteriophage, wherein preferably said coat protein comprises or preferably consists of the amino acid sequence of SEQ ID NO:3. In a further preferred embodiment said first attachment site is a lysine residue, wherein preferably said lysine residue is a lysine residue of a coat protein, preferably of a coat protein of an RNA bacteriophage, most preferably of RNA bacteriophage Qβ. In a further preferred embodiment said first attachment site is a lysine residue of the coat protein of RNA bacteriophage Qβ.

Attachment Site, Second: As used herein, the phrase “second attachment site” refers to an element which is naturally occurring with or which is artificially added to the IL-1 molecule and to which the first attachment site may be linked. The second attachment site of the IL-1 molecule preferably is a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is a sulfhydryl group. In a further preferred embodiment said second attachment site is a sulfhydryl group, preferably a sulfhydryl group of acysteine residue. The term “IL-1 molecule with at least one second attachment site” refers, therefore, to a construct comprising the IL-1 molecule and at least one second attachment site. However, in particular for a second attachment site, which is not naturally occurring within the IL-1 molecule, such a construct typically and preferably further comprises a “linker”. In another preferred embodiment the second attachment site is associated with the IL-1 molecule through at least one covalent bond, preferably through at least one peptide bond. In a further embodiment, the second attachment site is naturally occurring within the IL-1 molecule. In another further preferred embodiment, the second attachment site is artificially added to the IL-1 molecule, preferably through a linker, wherein further preferably said linker comprises or alternatively consists of a cysteine. Very preferably said linker is fused to the IL-1 molecule by way of a peptide bond.

Coat protein: The term “coat protein” refers to a viral protein, preferably a subunit of a natural capsid of a virus, preferably of an RNA bacteriophage, which is capable of being incorporated into a virus capsid or a VLP. Coat proteins are also known as capsid proteins.

Linked: The terms “linked” or “linkage” as used herein, refer to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only refer to a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker. Thus, in a preferred embodiment said least one first attachment site and said at least one second attachment site are covalently linked via least one, preferably exactly one, heterobifunctional cross-linker, wherein preferably said first attachment site is the amino group of a lysine residue and wherein further preferably said second attachment site is the sulfhydryl group of a cysteine residue. In other preferred embodiments the first attachment site and the second attachment site are linked through at least one covalent bond, preferably through at least one peptide bond, and even more preferably through exclusively peptide bond(s). In a very preferred embodiment the first attachment site and the second attachment site are linked exclusively by peptide bounds, preferably by genetic fusion, either directly, or, preferably, via an amino acid linker. In a further preferred embodiment the second attachment site is linked to the C-terminus of said first attachment site exclusively by peptide bounds, preferably by genetic fusion.

Linker: A “linker”, as used herein, either associates the second attachment site with the IL-1 molecule or comprises, essentially consists of, or consists of the second attachment site. Preferably, a “linker”, as used herein, comprises the second attachment site, typically and preferably—but not necessarily—as one amino acid residue, preferably as a cysteine residue. In a preferred embodiment said linker is an amino acid linker. In a very preferred embodiment said linker consists of exactly one cysteine residue. In a further preferred embodiment said linker comprises or consists of exactly one cysteine residue and said second attachment site is the sulthydryl group of said exactly one cysteine residue. Further linkers useful for the present invention are molecules comprising a C1-C6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl-moiety and additional amino acid(s) can also be used as linkers for the present invention and shall be encompassed within the scope of the invention. Association of the linker with the IL-1 molecule is preferably by way of at least one covalent bond, more preferably by way of at least one peptide bond. In the context of linkage by genetic fusion, a linker may be absent or preferably is an amino acid linker, more preferably an amino acid linker consisting exclusively of amino acid residues. Very preferred linkers for genetic fusion are flexible amino acid linkers. In the context of linkage by genetic fusion linkers preferred consist of 1 to 20, more preferably of 2 to 15, still more preferably of 2 to 10, still more preferably of 2 to 5, and most preferably of 3 amino acids. Very preferred linkers for genetic fusion comprise or preferably consist of GSG (SEQ ID NO:189).

Amino acid linker: The term “amino acid linker” refers to a linker comprising least one amino acid residue. Generally the term “amino acid linker” does not imply that such linker would consists exclusively of amino acid residues. However, in a preferred embodiment said amino acid linker consists exclusively of amino acid residues. The amino acid residues of the linker are, preferably, composed of naturally occurring amino acids or unnatural amino acids known in the art, all-L or all-D or mixtures thereof, most preferably all-L. Further preferred embodiments of a linker in accordance with this invention are molecules comprising a sulfhydryl group or a cysteine residue and such molecules are, therefore, also encompassed within this invention.

Ordered and repetitive antigen array: As used herein, the term “ordered and repetitive antigen array” generally refers to a repeating pattern of antigen or to a structure which is characterized by a typically and preferably high order of uniformity in spacial arrangement of the antigens with respect to virus-like particle, respectively. In one embodiment of the invention, the repeating pattern may be a geometric pattern. Certain embodiments of the invention, such as antigens coupled to the VLP of RNA bacteriophages, are typical and preferred examples of suitable ordered and repetitive antigen arrays which, moreover, possess strictly repetitive paracrystalline orders of antigens, preferably with spacings of 1 to 30 nanometers, preferably 2 to 15 nanometers, even more preferably 2 to 10 nanometers, even again more preferably 2 to 8 nanometers, and further more preferably 1.6 to 7 nanometers.

IL-1 molecule: The term “IL-1 molecule” or shortly “IL-1”, as used herein, refers to any polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:36 to SEQ ID NO:116, SEQ ID NO:130 to SEQ ID NO:140 and SEQ ID NO:163 to SEQ ID NO:165. The term “IL-1-molecule”, as used herein, preferably refers to any IL-1 protein, IL-1 fragment, IL-1 mature fragment, IL-1 peptide or IL-1 mutein comprising or alternatively consisting of a polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:36 to SEQ ID NO:116, SEQ ID NO:130 to SEQ ID NO:140 and SEQ ID NO:163 to SEQ ID NO:165. The term IL-1 molecule, as used herein, also typically and preferably refers to orthologs of IL-1 proteins of any animal species. An IL-1 molecule is preferably, but not necessarily, capable of binding to the IL-1 receptor and further preferably comprises biological activity.

IL-1 alpha molecule: The term “IL-1 alpha molecule” or shortly “IL-1 alpha”, as used herein, refers to an IL-1 alpha protein, IL-1 alpha fragment, IL-1 alpha mature fragment, IL-1 alpha peptide or IL-1 alpha mutein comprising or alternatively consisting of an polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:36 to 48, SEQ ID NO:63, SEQ ID NO:65, SEQ ID NO:67 to SEQ ID BO::88, and SEQ ID NO:163. A specifically preferred embodiment of IL-1 alpha is human IL-1 alpha 119-271 (SEQ ID NO:63).

IL-1 beta molecule: The term “IL-1 beta molecule” or shortly “IL-1 beta”, as used herein, refers to an IL-1 beta protein, IL-1 beta fragment, IL-1 beta mature fragment, IL-1 beta peptide or IL-1 beta mutein comprising or alternatively consisting of an polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:49 to SEQ ID NO:62, SEQ ID NO:64, SEQ ID NO:66, SEQ ID NO:89 to SEQ ID NO:116, SEQ ID NO:130 to SEQ ID NO:140, SEQ ID NO:164, and SEQ ID NO:165. A specifically preferred embodiment of IL-1 beta is human IL-1 beta 117-269 (SEQ ID NO:64).

IL-1 protein: The term “IL-1 protein”, as used herein, refers to a naturally occurring protein, wherein the amino acid sequence of said naturally occurring protein shows at least 80%, preferably at least 90%, more preferably at least 95%, even more preferably at least 99% and most preferably 100% sequence identity with any one of SEQ ID NO:36 to SEQ ID NO:62; or wherein said naturally occurring protein is capable of binding the IL-1 receptor and preferably comprises biological activity. The term “IL-1 protein”, as used herein, preferably refers to a naturally occurring protein, wherein the amino acid sequence of said naturally occurring protein shows at least 80%, preferably at least 90%, more preferably at least 95%, even more preferably at least 99% and most preferably 100% sequence identity with any one of SEQ ID NO:36 to SEQ ID NO:62; and wherein said naturally occurring protein is capable of binding the IL-1 receptor and preferably comprises biological activity. Typically and preferably, the term “IL-1 protein”, as used herein, refers to at least one naturally occurring protein, wherein said protein is capable of binding the IL-1 receptor and comprises biological activity, and wherein further said protein comprises or alternatively consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:36 to SEQ ID NO:62. Accordingly, the term “IL-1 alpha protein” relates to an IL-1 protein comprising or alternatively consisting of a polypeptide, wherein the amino acid sequence of said polypeptide shows at least 80%, preferably at least 90%, more preferably at least 95%, even more preferably at least 99% and most preferably 100% sequence identity with any one of SEQ ID NO:36 to SEQ ID NO:48, whereas the term “IL-1 beta protein” relates to an IL-1 protein comprising or alternatively consisting of a polypeptide, wherein the amino acid sequence of said polypeptide shows at least 80%, preferably at least 90%, more preferably at least 95%, even more preferably at least 99% and most preferably 100% sequence identity with any one of SEQ ID NO:49 to SEQ ID NO:62.

IL-1 fragment: The term “IL-1 fragment”, as used herein, relates to a polypeptide comprising a consecutive stretch of an IL-1 protein, wherein said polypeptide is at least 50, preferably at least 100, most preferably at least 150 amino acids in length. Typically and preferably said IL-1 fragment is at most 300, more preferably at most 250, and most preferably at most 200 amino acids in length. Typically and preferably, IL-1 fragments are capable of binding the IL-1 receptor and further preferably comprises biological activity. Accordingly, the terms “IL-1 alpha fragment” and “IL-1 beta fragment” relate to an IL-1 fragment as defined, wherein said IL-1 protein is an IL-1 alpha protein or an IL-1 beta protein, respectively.

IL-1 mature fragment: The term “IL-1 mature fragment”, as used herein, relates to a IL-1 fragment, wherein said IL-1 fragment is a naturally occurring maturation product of an IL-1 protein. Accordingly, the terms “IL-1 alpha mature fragment” and “IL-1 beta mature fragment”, as used herein relate to IL-1 mature fragments as defined, wherein said IL-1 protein is an IL-1 alpha protein or an IL-1 beta protein, respectively. Preferred embodiments of IL-1 alpha mature fragments are SEQ ID NO:63, SEQ ID NO:65 and SEQ ID NO:163. Preferred embodiments of IL-1 beta mature fragments are SEQ ID NO:64, SEQ ID NO:66, SEQ ID NO:130, SEQ ID NO:164, and SEQ ID NO:165.

Preferred IL-1 alpha mature fragments comprise or preferably consist of an amino acid sequence selected from the group consisting of: (a) human IL-1 alpha 119-271 (SEQ ID NO:63); (b) mouse IL-1 alpha 117-270 (SEQ ID NO:65); (c) mouse IL-1 alpha 117-270s (SEQ ID NO:163); and (e) an amino acid sequence which is at least 80%, or preferably at least 90%, more preferably at least 95%, or most preferably at least 99% identical with any one of SEQ ID NO:63, SEQ ID NO:65 and SEQ ID NO:163.

Preferred IL-1 beta mature fragments comprise or preferably consist of an amino acid sequence selected from the group consisting of: (a) human IL-1 beta 117-269 (SEQ ID NO:64); (b) human IL-1 beta 116-269 (SEQ ID NO:165); (c) mouse IL-1 beta 119-269 (SEQ ID NO:66); (d) mouse IL-1 beta 119-269s (SEQ ID NO:164); and (e) an amino acid sequence which is at least 80%, or preferably at least 90%, more preferably at least 95%, or most preferably at least 99% identical with any one of SEQ ID NO:64, SEQ ID NO:66, SEQ ID NO:164 and SEQ ID NO:165.

IL-1 peptide: The term “IL-1 peptide”, as used herein, relates to a polypeptide comprising a consecutive stretch of a naturally occurring protein, wherein said protein is capable of binding the IL-1 receptor and preferably comprises biological activity, wherein said polypeptide is 4 to 49, preferably 6 to 35, most preferably 10 to 25 amino acids in length. The IL-1 peptide may be, but typically is not, capable of binding the IL-1 receptor and typically has no biological activity. Accordingly, the terms “IL-1 alpha peptide” and “IL-1 beta peptide”, as used herein relate to IL-1 peptides as defined, wherein said naturally occurring protein is an IL-1 alpha protein or an IL-1 beta protein, respectively. Preferred IL-1 peptides are SEQ ID NO:82 to SEQ ID NO:116.

IL-1 mutein: The term “IL-1 mutein” as used herein comprise or preferably consist of any polypeptide derived from an IL-1 molecule, preferably from an IL-1 alpha or an IL-1 beta protein, an IL-1 alpha or an IL-1 beta fragment, an IL-1 alpha or an IL-1 beta mature fragment or an IL-1 alpha or an IL-1 beta peptide, wherein preferably said polypeptide exhibits reduced biological activity as compared to the IL-1 molecule it is derived from. Accordingly, IL-1 alpha muteins and IL-1 beta muteins are IL-1 muteins as defined, wherein said polypeptide is derived from an IL-1 alpha molecule or an IL-1 beta molecule, respectively. Very preferred IL-1 beta muteins are IL-1 beta muteins derived from IL-1 beta mature fragments, preferably from human IL-1β₁₁₇₋₂₆₉ (SEQ ID NO:64). Very preferred IL-1 alpha muteins are derived from IL-1 alpha mature fragments, preferably from human IL-1 α₁₁₉₋₂₇₁ (SEQ ID NO:63).

In preferred IL-1 muteins, said biological activity is less than 80%, more preferably less than 60%, still more preferably less than 40%, still more preferably less than 20% of the biological activity of the IL-1 molecule it is derived from, wherein further preferably said biological activity is determined by the capacity of said IL-1 mutein to induce IL-6 in human PBMCs, wherein most preferably said biological activity is determined essentially as described in Example 8B.

In preferred IL-1 beta muteins, said biological activity is less than 80%, more preferably less than 60%, still more preferably less than 40%, still more preferably less than 20% of the biological activity of the IL-1 beta molecule it is derived from, wherein preferably said IL-1 beta molecule is an IL-1 beta mature fragment, preferably human IL-1β₁₁₇₋₂₆₉ (SEQ ID NO:64), and wherein further preferably said biological activity is determined by the capacity of said IL-1 beta mutein to induce IL-6 in human PBMCs, wherein most preferably said biological activity is determined essentially as described in Example 8B.

In preferred IL-1 alpha muteins, said biological activity is less than 80%, more preferably less than 60%, still more preferably less than 40%, still more preferably less than 20% of the biological activity of the IL-1 alpha molecule it is derived from, wherein preferably said IL-1 alpha molecule is an IL-1 alpha mature fragment, preferably human human IL-1α₁₁₉₋₂₇₁ (SEQ ID NO:63), and wherein further preferably said biological activity is determined by the capacity of said IL-1 alpha mutein to induce IL-6 in human PBMCs, wherein most preferably said biological activity is determined essentially as described in Example 11.

Further preferred IL-1 muteins are derived from an IL-1 mature fragment, wherein the biological activity of said IL-1 mutein is less than 80%, more preferably less than 60%, still more preferably less than 40%, still more preferably less than 20% of the biological activity of the IL-1 mature fragment said IL-1 mutein is derived from. Very preferred IL-1 muteins do not exhibit biological activity, wherein preferably said biological activity is determined essentially as described in Examples 8B or 11.

Further preferably, but not necessarily, IL-1 muteins are capable of specifically binding an IL-1 receptor.

Compositions comprising a preferred IL-1 mutein as the sole antigen induce a titer of antibodies capable of specifically binding the IL-1 molecule said IL-1 mutein is derived from, wherein said titer is at least 20%, preferably at least 40%, still more preferably at least 60%, still more preferably at least 80% and most preferably at least 100% of the titer obtained with a composition comprising the IL-1 molecule said IL-1 mutein is derived from as the sole antigen, wherein preferably said titer is determined essentially as described in Example 9D.

When introduced into an animal, compositions comprising a preferred IL-1 beta mutein as the sole antigen induce a titer of antibodies capable of specifically binding the IL-1 beta molecule said IL-1 beta mutein is derived from, wherein preferably said IL-1 beta molecule is an IL-1 beta mature fragment, most preferably human IL-1β₁₁₇₋₂₆₉ (SEQ ID NO:64), wherein said titer is at least 20%, preferably at least 40%, still more preferably at least 60%, still more preferably at least 80% and most preferably at least 100% of the titer obtained with a composition comprising the IL-1 beta molecule said IL-1 beta mutein is derived from, preferably said IL-1 beta mature fragment, most preferably said human IL-1β₁₁₇₋₂₆₉ (SEQ ID NO:64) as the sole antigen, wherein further preferably said titer is determined essentially as described in Example 9D.

When introduced into an animal, compositions comprising a preferred IL-1 alpha mutein as the sole antigen induce a titer of antibodies capable of specifically binding the IL-1 alpha molecule said IL-1 alpha mutein is derived from, wherein preferably said IL-1 alpha molecule is an IL-1 alpha mature fragment, most preferably human IL-1β₁₁₉₋₂₇₁ (SEQ ID NO:63), wherein said titer is at least 20%, preferably at least 40%, still more preferably at least 60%, still more preferably at least 80% and most preferably at least 100% of the titer obtained with a composition comprising the IL-1 alpha molecule said IL-1 alpha mutein is derived from, preferably said IL-1 alpha mature fragment, most preferably said human IL-1 α₁₁₉₋₂₇₁ (SEQ ID NO:63) as the sole antigen, wherein further preferably said titer is determined essentially as described in Example 9D.

A very preferred IL-1 mutein is an IL-1 mutein, wherein said biological activity is less than 80%, more preferably less than 60%, still more preferably less than 40%, still more preferably less than 20% of the biological activity of the IL-1 molecule it is derived from, wherein further preferably said biological activity is determined by the capacity of said IL-1 mutein to induce IL-6 in human PBMCs, wherein most preferably said biological activity is determined essentially as described in Example 8B, and wherein additionally compositions comprising said very preferred IL-1 mutein as the sole antigen induce a titer of antibodies capable of specifically binding the IL-1 molecule said very preferred IL-1 mutein is derived from, wherein said titer is at least 20%, preferably at least 40%, still more preferably at least 60%, still more preferably at least 80% and most preferably at least 100% of the titer obtained with a composition comprising the IL-1 molecule said very preferred IL-1 mutein is derived from as the sole antigen, wherein preferably said titer is determined essentially as described in Example 9D.

Very preferred are IL-1 muteins derived from (i) an IL-1 protein, preferably from SEQ ID NO:36 to SEQ ID NO:62; or (ii) more preferably of an IL-1 mature fragment, preferably from any one of SEQ ID NO:63 to SEQ ID NO:66, SEQ ID NO:130, and SEQ ID NO:163 to SEQ ID NO:165.

IL-1 muteins useful in the context of the invention have been described in Kamogashira et al. (1988) J. Biochem. 104:837-840; Gehrke et al. (1990) The Journal of Biological Chemistry 265(11):5922-5925; Conca et al. (1991) The Journal of Biological Chemistry 266(25):16265-16268; Ju et al. (1991) PNAS 88:2658-2662; Auron et al. (1992) Biochemistry 31:6632-6638; Guinet et al. (1993) Eur. J. Biochem 211:583-590; Camacho (1993) Biochemistry 32:8749-8757; Baumann (1993) Journal of Recepror Research 13(1-4):245-262; Simon (1993) The Journal of Biological Chemistry 268(13):9771-9779; and Simoncsits (1994) Cytokine 6(2):206-214, the disclosure of which is incorporated herein by reference.

Preferred IL-1 muteins comprise or preferably consist of a polypeptide, wherein the amino acid sequence of said polypeptide differs from the amino acid sequence of an IL-1 protein, an IL-1 fragment, an IL-1 mature fragment or an IL-1 peptide in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). In a preferred embodiment, said amino acid residues are in one consecutive stretch. Further preferred IL-1 muteins comprise or preferably consist of a polypeptide, wherein the amino acid sequence of said polypeptide differs from the amino acid sequence of an IL-1 protein, an IL-1 fragment, or an IL-1 mature fragment, preferably of an IL-1 mature fragment, in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii).

Further preferred IL-1 muteins comprise or more preferably consist of a polypeptide, wherein the amino acid sequence of said polypeptide differs from an amino acid sequence selected from SEQ ID NO:36 to SEQ ID NO:48 and SEQ ID NO:49 to SEQ ID NO:62 in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). Further preferred IL-1 muteins comprise or preferably consist of a polypeptide, wherein the amino acid sequence of said polypeptide differs from an amino acid sequence selected from the group consisting of (i) any one of SEQ ID NO:63, SEQ ID NO:65, and SEQ ID NO:163, most preferably SEQ ID NO:63; or (ii) any one of SEQ ID NO:64, SEQ ID NO:66, SEQ ID NO:130, SEQ ID NO:164, and SEQ ID NO:165, most preferably SEQ ID NO:64; in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii).

Further preferred IL-1 muteins are IL-1 alpha muteins, wherein said IL-1 alpha muteins comprise or more preferably consist of a polypeptide, wherein the amino acid sequence of said polypeptide differs from an amino acid sequence selected from SEQ ID NO:36 to SEQ ID NO:48 in 1 to 6, preferably 1 to 5, more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). Further preferred IL-1 alpha muteins comprise or preferably consist of a polypeptide, wherein the amino acid sequence of said polypeptide differs from an amino acid sequence selected from SEQ ID NO:63, SEQ ID NO:65, and SEQ ID NO:163, most preferably SEQ ID NO:63, in 1 to 6, preferably 1 to 5, more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii).

Very preferred IL-1 alpha muteins comprise or preferably consist of a polypeptide, wherein the amino acid sequence of said polypeptide differs from the amino acid sequence of SEQ ID NO:63 in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). Still more preferred IL-1 alpha muteins comprise or preferably consist of a polypeptide, wherein the amino acid sequence of said polypeptide is selected from the group consisting of SEQ ID NO:210 to SEQ ID NO:218.

Further preferred IL-1 muteins are IL-1 beta muteins, wherein said IL-1 beta muteins comprise or more preferably consist of a polypeptide, wherein the amino acid sequence of said polypeptide differs from an amino acid sequence selected from SEQ ID NO:49 to SEQ ID NO:62 in 1 to 6, preferably 1 to 5, more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). Further preferred IL-1 beta muteins comprise or preferably consist of a polypeptide, wherein the amino acid sequence of said polypeptide differs from an amino acid sequence selected from SEQ ID NO:64, SEQ ID NO:66, SEQ ID NO:130, SEQ ID NO:164, and SEQ ID NO:165, most preferably SEQ ID NO:64, in 1 to 6, preferably 1 to 5, more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). Very preferred IL-1 beta muteins comprise or preferably consist of a polypeptide, wherein the amino acid sequence of said polypeptide differs from the amino acid sequence of SEQ ID NO:64 in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) deleted from said polypeptide, (ii) inserted into said polypeptide, (iii) exchanged by another amino acid residue, or (iv) any combination of (i) to (iii). Still more preferred IL-1 beta muteins comprise or preferably consist of a polypeptide, wherein the amino acid sequence of said polypeptide is selected from SEQ ID NO:131 to SEQ ID NO:140 and SEQ ID NO:205 to SEQ ID NO:209.

“derived from”: in the context of the invention, the expression an amino acid sequence which is “derived from” another amino acid sequence means that said amino acid sequence is essentially identical with the amino acid sequence it is derived from, with the exception of certain mutations, wherein said mutations are selected from the group consisting of (i) amino acid exchanges, (ii) deletions, (iii) insertions, and (iv) any combination of (i) to (iii), wherein preferably said mutations are selected from (i) amino acid exchanges and (ii) deletions. In particular, a mutated amino acid sequence derived from a wild type amino acid sequence preferably differs from said wild type amino acid sequence in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) exchanged by another amino acid, (ii) deleted from said wild type amino acid, (iii) inserted into said wild type sequence, and (iv) any combination of (i) to (iii), wherein most preferably said amino acid residue(s) are (i) exchanged by another amino acid, or (ii) deleted from said wild type amino acid. Deletions of more than one amino acid residue preferably occur as a deletion of a consecutive stretch of amino acid residues of said wild type amino acid sequence. A mutated amino acid sequence which is derived from a wild type amino acid sequence preferably has at least 90%, 91%, 92%, 93%, 94%, 95%, 96%, 97%, 98%, and most preferably at least 99% sequence identity with said wild type amino acid sequence.

Similarly, the expression “mutein derived from a IL-1 molecule” refers to a mutein, wherein said mutein comprises or preferably consists of a polypeptide, wherein the amino acid sequence of said polypeptide is essentially identical to that of the IL-1 molecule it is derived from, with the exception of certain mutations, wherein said mutations are selected from the group consisting of (i) amino acid exchanges, (ii) deletions, (iii) insertions, and (iv) any combination of (i) to (iii), wherein preferably said mutations are selected from (i) amino acid exchanges and (ii) deletions. In particular, an IL-1 mutein derived from an IL-1 molecule differs from said IL-1 molecule in 1 to 10, preferably 1 to 6, more preferably 1 to 5, still more preferably 1 to 4, still more preferably 1 to 3, still more preferably 1 to 2, and most preferably in exactly 1 amino acid residue(s), wherein preferably said amino acid residue(s) are (i) exchanged by another amino acid, (ii) deleted from said wild type amino acid, (iii) inserted into said wild type sequence, and (iv) any combination of (i) to (iii), wherein most preferably said amino acid residue(s) are (i) exchanged by another amino acid, or (ii) deleted from said wild type amino acid. Deletions of more than one amino acid residue preferably occur as a deletion of a consecutive stretch of amino acid residues of the IL-1 molecule said IL-1 mutein is derived from. A mutein derived from a wild type amino acid sequence preferably has at least 90%, 91%, 92%, 93%, 94%, 95%, 96%, 97%, 98%, and most preferably at least 99% sequence identity with the IL-1 molecule said IL-1 mutein is derived from.

Amino acid exchange: the expression amino acid exchange refers to the exchange of an amino acid residue in a certain position of an amino acid sequence by any other amino acid residue.

Agonistic effect/biological activity of the IL-1: The terms “biological activity” or “biologically active” as used herein with respect to IL-1 refer to the ability of the IL-1 molecule to induce the production of IL-6 after systemical administration into animals, preferably as outlined in Example 2E. and in Example 3E. By biological activity of the IL-1 molecule is also meant the ability to induce the proliferation of thymocytes (Epps et al., Cytokine 9(3):149-156 (1997), D10.G4.1 T helper cells (Orencole and Dinarello, Cytokine 1(1):14-22 (1989), or the ability to induce the production of IL-6 from MG64 or HaCaT cells (Boraschi et al., J. Immunol. 155:4719-4725 (1995) or fibroblasts (Dinarello et al., Current Protocols in Immunology 6.2.1-6-2-7 (2000)), or the production of IL-2 from EL-4 thymoma cells (Simon et al., J. Immunol. Methods 84(1-2):85-94 (1985)), or the ability to inhibit the growth of the human melanoma cell line A375 (Nakai et al., Biochem. Biophys. Res. Commun. 154:1189-1196 (1988)). Very preferably, the term biological activity of an IL-1 molecule or an IL-1 mutein refers to the capacity of an IL-1 composition comprising said IL-1 molecule or said IL-1 mutein to induce IL-6 in human PBMCs, wherein preferably said IL-1 molecule or said IL-1 mutein is the sole antigen in said IL-1 composition, and wherein most preferably said biological activity is determined essentially as described in Example 8B.

Packaged: The term “packaged” as used herein refers to the state of a polyanionic macromolecule or of an immunostimulatory substances in relation to the VLP. The term “packaged” as used herein includes binding that may be covalent, e.g., by chemically coupling, or non-covalent, e.g., ionic interactions, hydrophobic interactions, hydrogen bonds, etc. In a preferred embodiment the term “packaged” refers to the enclosement, or partial enclosement, of a polyanionic macromolecule by the VLP. Thus, the polyanionic macromolecule or immunostimulatory substances can be enclosed by the VLP without the existence of an actual binding, in particular of a covalent binding. In preferred embodiments, the at least one polyanionic macromolecule or immunostimulatory substances is packaged into the VLP, most preferably in a non-covalent manner. Methods for packaging polyanionic macromolecules such as polyglutamic acid into VLPs, and in particular into VLPs of RNA bacteriophages, are disclosed in WO2006/037787. Reference is made in particular to Example 4 of WO2006/037787. Methods for packaging immunostimulatory substances, preferably immunostimulatory nucleic acids, and most preferably unmethylated CpG-containing oligonucleotides into a VLP are described in WO2003/024481A2. In case said immunostimulatory substances is nucleic acid, preferably a DNA, and most preferably an unmethylated CpG-containing oligonucleotide, the term packaged implies that said nucleic acid is not accessible to nucleases hydrolysis, preferably not accessible to DNAse hydrolysis (e.g. DNaseI or Benzonase), wherein preferably said accessibility is assayed as described in Examples 11-17 of WO2003/024481A2.

Polyanionic macromolecule: The term “polyanionic macromolecule”, as used herein, refers to a molecule of high relative molecular mass which comprises repetitive groups of negative charge, the structure of which essentially comprises the multiple repetition of units derived, actually or conceptually, from molecules of low relative molecular mass. The term “polyanionic macromolecule” as used herein refers to a molecule that is not capable of activating toll-like receptors. Thus, the term “polyanionic macromolecule” excludes Toll-like receptors ligands, and excludes substances capable of inducing and/or enhancing an immune response, such as Toll-like receptors ligands, nucleic acids capable of inducing and/or enhancing an immune response, and lipopolysacchrides (LPS). More preferably the term “polyanionic macromolecule” as used herein, refers to a molecule that is not capable of inducing cytokine production. Preferably, polyanionic macromolecules are polyanionic polypeptides or anionic dextrans. In a preferred embodiment said polyanionic macromolecules are polyanionic polypeptides, wherein preferably said polyanionic polypeptides are selected from a group consisting of: (a) polyglutamic acid; (b) polyaspartic acid; (c) poly(GluAsp) and (d) any chemical modifications of (a) to (c). Examples for chemical modifications include, but are not limited to glycosylations, acetylations, and phosphorylations. In a further preferred embodiment said polyanionic macromolecules are anionic dextrans selected from a group consisting of: (a) dextran sulfate; (b) carboxylmethyl dextran; (c) sulfopropyl dextran; (d) methyl sulfonate dextran; and (e) dextrane phosphate.

Polyaspartic acid: The term “polyaspartic acid” as used herein, refers to a polypeptide comprising at least 50%, preferably at least 70%, more preferably at least 90%, more preferably at least 95%, more preferably at least 99%, more preferably 100%, aspartic acid residues out of the total number of amino acid residues comprised by said polypeptide. The aspartic acid residues of said polypeptide are hereby either all-L, all-D, or mixtures of L- and D-aspartic acid. Most preferably said polypeptide only comprises L-aspartic acid residues.

Polyglutamic acid: The term “polyglutamic acid”, as used herein, refers to a polypeptide comprising at least 50%, preferably at least 70%, more preferably at least 90%, more preferably at least 95%, more preferably at least 99%, and most preferably 100% glutamic acid residues out of the total number of amino acid residues comprised by said polypeptide. The glutamic acid residues of said polypeptide are hereby either all-L, all-D, or mixtures of L- and D-glutamic acid. Most preferably said polypeptide only comprises L-glutamic acid residues.

Poly (GluAsp): The term “Poly (GluAsp)” as used herein, refers to a polypeptide comprising at least 50%, preferably at least 70%, more preferably at least 90%, still more preferably at least 95%, still more preferably at least 99%, and most preferably 100% glutamic acid residues and aspartic acid residues, out of the total number of amino acid residues comprised by said polypeptide. The glutamic acid molecules and the aspartic acid molecules are hereby either all-L or all-D or mixtures thereof. Most preferably said polypeptide only comprises L-glutamic acid residues and L-aspartic acid residues.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

Recombinant VLP: The term “recombinant VLP”, as used herein, refers to a VLP that is obtained by a process which comprises at least one step of recombinant DNA technology.

Virus particle: The term “virus particle” as used herein refers to the morphological form of a virus. In some virus types it comprises a genome surrounded by a protein capsid; others have additional structures (e.g., envelopes, tails, etc.).

Virus-like particle (VLP), as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA bacteriophage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular. For example, the capsids of RNA bacteriophages or HBcAgs have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness. One common feature of virus particle and virus-like particle is its highly ordered and repetitive arrangement of its subunits.

Virus-like particle of an RNA bacteriophage: As used herein, the term “virus-like particle of an RNA bacteriophage” refers to a virus-like particle comprising, or preferably consisting essentially of, or consisting of coat proteins, mutants or fragments thereof, of an RNA bacteriophage. In addition, a virus-like particle of an RNA bacteriophage is resembling the structure of an RNA bacteriophage. Furthermore, a virus-like particle of an RNA bacteriophage is non replicative and/or non-infectious. Typically and preferably a virus-like particle of an RNA bacteriophage is lacking at least one of the genes, preferably all genes, encoding the replication machinery of the RNA bacteriophage. Further preferably a virus-like particle of an RNA bacteriophage is also lacking the gene or genes encoding the protein or proteins responsible for viral attachment to or entry into the host. This definition, however, also encompasses virus-like particles of RNA bacteriophages, in which the aforementioned gene or genes are still present but inactive, and, therefore, also are leading to non-replicative and/or non-infectious virus-like particles of an RNA bacteriophage. Preferred VLPs derived from RNA bacteriophages exhibit icosahedral symmetry and consist of 180 subunits (monomers). Preferred methods to render a virus-like particle of an RNA bacteriophage non replicative and/or non-infectious is by physical, chemical inactivation, such as UV irradiation, formaldehyde treatment, typically and preferably by genetic manipulation.

One, a, or an: when the terms “one”, “a”, or “an” are used in this disclosure, they mean “at least one” or “one or more” unless otherwise indicated.

diabetes: The term diabetes refers to any type of diabetes mellitus. Preferably diabetes refers to type I diabetes and/or type II diabetes. Most preferably diabetes refers to type II diabetes.

The compositions described herein are capable of inducing or enhancing an immune responses against IL-1 in an animal or in human. It has surprisingly been found that immunization with an IL-1 molecule, i.e. with an IL-1 alpha molecule or with an IL-1 beta molecule, or with a combination of both, resulted in a clear amelioration of the diet-induced diabetic phenotype in male C57BL/6 mice.

The invention therefore provides a composition for the treatment, amelioration or prophylaxis of diabetes, preferably of type II diabetes, wherein said composition comprises: (a) a core particle with at least one first attachment site, wherein said core particle is a virus-like particle (VLP) or a virus particle, preferably a virus-like particle; and (b) at least one antigen with at least one second attachment site, wherein the at least one antigen comprises or consists of an IL-1 molecule, preferably selected from the group consisting of IL-1 protein, IL-1 mature fragment, IL-1 peptide and IL-1 mutein, wherein (a) and (b) are covalently linked through the at least one first and the at least one second attachment site.

In a preferred embodiment, said composition comprises (a) a virus-like particle (VLP) with at least one first attachment site; and (b) at least one antigen with at least one second attachment site; wherein said at least one antigen comprises an IL-1 molecule and wherein (a) and (b) are linked through said at least one first and said at least one second attachment site. In a further preferred embodiment said at least one antigen with at least one second attachment site comprises or preferably consists of (i) an IL-1 molecule; and (ii) a linker, wherein preferably said linker comprises or preferably consists of said second attachment site.

Preferably, said IL-1 molecule is linked to the core particle, so as to form an ordered and repetitive antigen-VLP array. In preferred embodiments of the invention, at least 20, preferably at least 30, more preferably at least 60, again more preferably at least 120 and further more preferably at least 180 IL-1 molecules are linked to the core particle.

Any virus known in the art having an ordered and repetitive structure may be selected as a VLP or a virus particle of the invention. Illustrative DNA or RNA viruses, the coat or capsid protein of which can be used for the preparation of VLPs have been disclosed in WO 2004/009124 on page 25, line 10-21, on page 26, line 11-28, and on page 28, line 4 to page 31, line 4. These disclosures are incorporated herein by way of reference.

Virus particles or virus-like particles can be produced and purified from virus-infected cell cultures. The resulting virus particles or virus-like particles for vaccine purpose should be preferably non-replicative or non-infectious, more preferably non-replicative and non-infectious. UV irradiation, chemical treatment, such as with formaldehyde or chloroform, are the general methods known to skilled person in the art to inactivate virus.

In one preferred embodiment, the core particle is a virus particle, wherein preferably said virus particle is a bacteriophage, and wherein further preferably said bacteriophage is an RNA bacteriophage, and wherein still further preferably said RNA bacteriophage is an RNA bacteriophage selected from Qβ, fr, GA or AP205.

In one preferred embodiment, the core particle is a VLP. In a further preferred embodiment, the VLP is a recombinant VLP Almost all commonly known viruses have been sequenced and are readily available to the public. The gene encoding the coat protein can be easily identified by a skilled artisan. The preparation of VLPs by recombinantly expressing the coat protein in a host is within the common knowledge of the artisan.

In one preferred embodiment, the virus-like particle comprises, or alternatively consists of, recombinant proteins, mutants or fragments thereof, of a virus selected form the group consisting of: (a) RNA bacteriophages; (b) bacteriophages; (c) Hepatitis B virus, preferably its capsid protein (Ulrich, et al., Virus Res. 50:141-182 (1998)) or its surface protein (WO 92/11291); (d) measles virus (Warnes, et al., Gene 160:173-178 (1995)); (e) Sindbis virus; (f) rotavirus (U.S. Pat. No. 5,071,651 and U.S. Pat. No. 5,374,426); (g) foot-and-mouth-disease virus (Twomey, et al., Vaccine 13:1603 1610, (1995)); (h) Norwalk virus (Jiang, X., et al., Science 250:1580 1583 (1990); Matsui, S. M., et al., J. Clin. Invest. 87:1456 1461 (1991)); (i) Alphavirus; (j) retrovirus, preferably its GAG protein (WO 96/30523); (k) retrotransposon Ty, preferably the protein p1; (l) human Papilloma virus (WO 98/15631); (m) Polyoma virus, preferably BKV; (n) Tobacco mosaic virus; and (o) Flock House Virus.

A VLP comprising more than one species of recombinant protein is generally referred, in this application, as mosaic VLP. In one embodiment, the VLP is a mosaic VLP, wherein said mosaic VLP comprises, or consists of, more than one recombinant protein species, preferably of two different recombinant proteins, most preferably of two different recombinant capsid proteins, mutants or fragments thereof.

The term “fragment of a recombinant protein” or the term “fragment of a coat protein”, as used herein, is defined as a polypeptide, which is at least 70%, preferably at least 80%, more preferably at least 90%, even more preferably at least 95% of the length of the wild-type recombinant protein, or coat protein, respectively, and which preferably retains the capability of forming a VLP. Preferably, the fragment is obtained by at least one internal deletion, at least one truncation or at least one combination thereof. Further preferably, the fragment is obtained by at most 5, 4, 3 or 2 internal deletions, by at most 2 truncations or by exactly one combination thereof.

The term “fragment of a recombinant protein” or “fragment of a coat protein” shall further refer to a polypeptide, which has at least 80%, preferably at least 90%, more preferably at least 95% amino acid sequence identity with the “fragment of a recombinant protein” or “fragment of a coat protein”, respectively, as defined above and which is preferably capable of assembling into a virus-like particle.

The term “mutant coat protein” refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence, and wherein preferably said amino acid sequence retains the ability to assemble into a VLP.

In one preferred embodiment, the virus-like particle is a virus like particle of Hepatitis B virus. The preparation of Hepatitis B virus-like particles has been disclosed, inter alia, in WO 00/32227, WO 01/85208 and in WO 01/056905. All three documents are explicitly incorporated herein by way of reference. Other variants of HBcAg suitable for use in the practice of the present invention have been disclosed in page 34-39 of WO 01/056905.

In one further preferred embodiment of the invention, a lysine residue is introduced into the HBcAg polypeptide, to mediate the linking of IL-1 molecule to the VLP of HBcAg. In preferred embodiments, VLPs and compositions of the invention are prepared using a HBcAg comprising, or alternatively consisting of, amino acids 1-144, or 1-149, 1-185 of SEQ ID NO:1, which is modified so that the amino acids at positions 79 and 80 are replaced with a peptide having the amino acid sequence of Gly-Gly-Lys-Gly-Gly (SEQ ID NO:170). This modification changes the SEQ ID NO:1 to SEQ ID NO:2. In further preferred embodiments, the cysteine residues at positions 48 and 110 of SEQ ID NO:2, or its corresponding fragments, preferably 1-144 or 1-149, are mutated to serine. The invention further includes compositions comprising Hepatitis B core protein mutants having above noted corresponding amino acid alterations. The invention further includes compositions and vaccines, respectively, comprising HBcAg polypeptides which comprise, or alternatively consist of, amino acid sequences which are at least 80%, 85%, 90%, 95%, 97% or 99% identical to SEQ ID NO:2.

In one preferred embodiment of the invention, the virus-like particle comprises, consists essentially of, or alternatively consists of, recombinant coat proteins, mutants or fragments thereof, of an RNA bacteriophage. Preferably, the RNA bacteriophage is selected from the group consisting of (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (l) bacteriophage PP7; (m) bacteriophage PRR1, and (n) bacteriophage AP205.

In one preferred embodiment of the invention, the virus-like particle comprises coat proteins, mutants or fragments thereof, of RNA bacteriophages, wherein said coat proteins comprise or preferably consists of an amino acid sequence selected from the group consisting of: (a) SEQ ID NO:3, referring to Qβ CP; (b) a mixture of SEQ ID NO:3 and SEQ ID NO:4 (QβA1 protein); (c) SEQ ID NO:5 (R17 capsid protein); (d) SEQ ID NO:6 (fr capsid protein); (e) SEQ ID NO:7 (GA capsid protein); (f) SEQ ID NO:8 (SP capsid protein); (g) a mixture of SEQ ID NO:8 and SEQ ID NO:9; (h) SEQ ID NO:10 (MS2 capsid protein); (i) SEQ ID NO:11 (M11 capsid protein); (j) SEQ ID NO:12 (MX1 capsid protein); (k) SEQ ID NO:13 (NL95 capsid protein); (l) SEQ ID NO:14 (f2 capsid protein); (m) SEQ ID NO:15 (PP7 capsid protein); and (n) SEQ ID NO:21 (AP205 capsid protein).

In one preferred embodiment of the invention, the VLP is a mosaic VLP comprising or alternatively consisting of more than one amino acid sequence, preferably two amino acid sequences, of coat proteins, mutants or fragments thereof, of an RNA bacteriophage. In one very preferred embodiment, the VLP comprises or alternatively consists of two different coat proteins of an RNA bacteriophage, wherein said two different coat proteins comprise or preferably consist of the amino acid sequence of CP Qβ (SEQ ID NO: 3) and CP Qβ A1 (SEQ ID NO:4); or of the amino acid sequence of CP SP (SEQ ID NO:8) and CP SP A1 (SEQ ID NO:9).

In preferred embodiments of the present invention, the virus-like particle comprises, or alternatively consists essentially of, or alternatively consists of recombinant coat proteins, mutants or fragments thereof, of an RNA bacteriophage, wherein preferably said RNA bacteriophage is selected from bacteriophage Qβ, bacteriophage fr, bacteriophage AP205, and bacteriophage GA.

In one preferred embodiment, the VLP is a VLP of RNA bacteriophage Qβ. The capsid or virus-like particle of Qβ shows an icosahedral phage-like capsid structure with a diameter of 25 nm and T=3 quasi symmetry. The capsid contains 180 copies of the coat protein, which are linked in covalent pentamers and hexamers by disulfide bridges (Golmohammadi, R. et al., Structure 4:543-5554 (1996)), leading to a remarkable stability of the Qβ capsid. Capsids or VLPs made from recombinant Qβ coat protein may contain, however, subunits not linked via disulfide bonds to other subunits within the capsid, or incompletely linked. The capsid or VLP of Qβ shows unusual resistance to organic solvents and denaturing agents. Surprisingly, we have observed that DMSO and acetonitrile concentrations as high as 30%, and guanidinium concentrations as high as 1 M do not affect the stability of the capsid. The high stability of the capsid or VLP of Qβ is an advantageous feature, in particular, for its use in immunization and vaccination of mammals and humans in accordance of the present invention.

Further preferred virus-like particles of RNA bacteriophages, in particular of RNA bacteriophage Qβ and RNA bacteriophage fr, are disclosed in WO 02/056905, the disclosure of which is herewith incorporated by reference in its entirety. In particular, Example 18 of WO 02/056905 provides a detailed description of the preparation of VLPs of RNA bacteriophage Qβ.

In another preferred embodiment, the VLP is a VLP of RNA bacteriophage AP205. Assembly-competent mutant forms of AP205 VLPs, including AP205 coat protein with the substitution of proline at amino acid 5 to threonine or, AP205 coat protein with the substitution of asparagine to aspartic acid at amino acid 14 may also be used in the practice of the invention and leads to other preferred embodiments of the invention. WO 2004/007538 describes, in particular in Example 1 and Example 2, how to obtain VLP comprising AP205 coat proteins, and hereby in particular the expression and the purification thereof. WO 2004/007538 is incorporated herein by way of reference. AP205 VLPs are highly immunogenic, and can be linked with the antigen to typically and preferably generate vaccine constructs displaying the IL-1 molecule oriented in a repetitive manner.

In one preferred embodiment, the VLP comprises, essentially consists of, or alternatively consists of a mutant coat protein of a virus, preferably of an RNA bacteriophage, wherein the mutant coat protein has been modified by removal of at least one lysine residue by way of substitution and/or by way of deletion. In another preferred embodiment, the VLP comprises, essentially consists of, or alternatively consists of a mutant coat protein of a virus, preferably an RNA bacteriophage, wherein said mutant coat protein has been modified by addition of at least one lysine residue by way of substitution and/or by way of insertion. The deletion, substitution or addition of at least one lysine residue allows varying the degree of coupling, i.e. the amount of IL-1 molecules per subunits of the VLP, preferably of the VLP of an RNA bacteriophage, in particular, to match and tailor the requirements of the vaccine.

In one preferred embodiment, the compositions and vaccines of the invention have an antigen density being from 0.5 to 4.0. The term “antigen density”, as used herein, refers to the average number of IL-1 molecules which is linked per subunit, preferably per coat protein, of the VLP, and hereby preferably of the VLP of an RNA bacteriophage. Thus, this value is calculated as an average over all the subunits of the VLP, preferably of the VLP of the RNA bacteriophage, in the composition or vaccines of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. Preferably, the at least one first attachment site is a lysine residue, pointing to or being on the exterior of the VLP. In a further preferred embodiment said first attachment site is an amino group of a lysine residue of SEQ ID NO:3. In a further preferred embodiment said first attachment site is the amino group of any one of the lysine residues in positions 2, 13, 16, 46, 60, 63, and 67 of SEQ ID NO:3. In a further preferred embodiment said first attachment site is the amino group of any one of the lysine residues of the coat protein, preferably of SEQ ID NO:3, which are exposed to the exterior of the capsid.

Qβ mutants, of which exposed lysine residues are replaced by arginines can be used for the present invention. Thus, in another preferred embodiment of the present invention, the virus-like particle comprises, consists essentially of, or alternatively consists of mutant Qβ coat proteins. Preferably these mutant coat proteins comprise or alternatively consist of an amino acid sequence selected from the group of (a) Qβ-240 (SEQ ID NO:16, Lys13-Arg of SEQ ID NO: 3); (b) Qβ-243 (SEQ ID NO:17, Asn10-Lys of SEQ ID NO:3); (c) Qβ-250 (SEQ ID NO:18, Lys2-Arg of SEQ ID NO:3); (d) Qβ-251 (SEQ ID NO:19, Lys16-Arg of SEQ ID NO:3); and (e) Qβ-259 (SEQ ID NO:20, Lys2-Arg, Lys16-Arg of SEQ ID NO:3). The construction, expression and purification of the above indicated Qβ mutant coat proteins, mutant Qβ coat protein VLPs and capsids, respectively, are described in WO 02/056905. In particular is hereby referred to Example 18 of above mentioned application.

In another preferred embodiment of the present invention, the virus-like particle comprises, or alternatively consists essentially of, or alternatively consists of mutant coat protein of Qβ, or of fragments thereof, and the corresponding A1 protein. In a further preferred embodiment, the virus-like particle comprises, or alternatively consists essentially of, or alternatively consists of mutant coat protein with amino acid sequence SEQ ID NO:16, 17, 18, 19, or 20 and the corresponding A1 protein.

Further RNA bacteriophage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Priano, C. et al., J. Mol. Biol. 249:283-297 (1995)). In particular the biological and biochemical properties of GA (Ni, C Z., et al., Protein Sci. 5:2485-2493 (1996), Tars, K et al., J. Mol. Biol. 271:759-773 (1997)) and of fr (Pushko P. et al., Prot. Eng. 6:883-891 (1993), Liljas, L et al. J. Mol. Biol. 244:279-290, (1994)) have been disclosed. The crystal structure of several RNA bacteriophages has been determined (Golmohammadi, R. et al., Structure 4:543-554 (1996)). Using such information, surface exposed residues can be identified and, thus, RNA bacteriophage coat proteins can be modified such that one or more reactive amino acid residues can be inserted by way of insertion or substitution. Another advantage of the VLPs derived from RNA bacteriophages is their high expression yield in bacteria that allows production of large quantities of material at affordable cost.

In one preferred embodiment, the composition of the invention comprises at least one antigen, preferably one to four, more preferably one to three, still more preferably one to two and most preferably exactly one antigen, wherein said antigen comprises or consists of an IL-1 molecule, preferably an IL-1 protein, an IL-1 fragment, an IL-1 mature fragment, an IL-1 peptide or an IL-1 mutein, wherein said IL-1 molecule preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:36 to SEQ ID NO:116, SEQ ID NO:130 to SEQ ID NO:140 and SEQ ID NO:163 to SEQ ID NO:165.

In a further preferred embodiment said antigen comprises or consists of an IL-1 molecule derived from an organism selected from the group consisting of: (a) humans; (b) primates; (c) rodents; (d) horses; (e) sheep; (f) cat; (g) cattle; (h) pig; (i) rabbit; (j) dog; (k) mouse; and (l) rat. Most preferably said IL-1 molecule is derived from humans. In a very preferred embodiment, the IL-1 molecule is a human IL-1 molecule. Further preferably said IL-1 molecule comprises or preferably consists of a polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:36, SEQ ID NO:49, SEQ ID NO:63, SEQ ID NO:64, any one of SEQ ID NO:67 to 110, and one of SEQ ID NO:130-140, and SEQ ID NO:165.

In a further preferred embodiment said IL-1 molecule is derived from rat or mouse, preferably mouse, wherein said IL-1 molecule preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:45, SEQ ID NO:46, SEQ ID NO:53, SEQ ID NO:54, SEQ ID NO:65, SEQ ID NO:66, any one of SEQ ID NO:111 to SEQ ID NO:116, SEQ ID NO:163, and SEQ ID NO:164.

In a further preferred embodiment said IL-1 molecule is an IL-1 alpha molecule, preferably an IL-1 alpha protein, an IL-1 alpha fragment, an IL-1 alpha mature fragment, an IL-1 alpha peptide or an IL-1 alpha mutein, wherein said IL-1 alpha molecule preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:36 to 48, SEQ ID NO:63, SEQ ID NO:65, SEQ ID NO:67 to 88, and SEQ ID NO:165. Specifically preferred embodiments of IL-1 alpha molecules are human IL-1 alpha molecules, preferably human IL-1 alpha proteins, human IL-1 alpha fragments or human IL-1 alpha mature fragments, wherein said IL-1 alpha molecules preferably comprise or even more preferably consist of a polypeptide, wherein the amino acid sequence of said polypeptide shows at least 80%, preferably at least 90%, more preferably at least 95%, even more preferably at least 99% and most preferably 100% sequence identity with any one of SEQ ID NO:36, SEQ ID NO:63, and SEQ ID NO:163, most preferably SEQ ID NO:63.

In a further preferred embodiment said IL-1 molecule is an IL-1 beta molecule, preferably an IL-1 beta protein, an IL-1 beta fragment, an IL-1 beta mature fragment, an IL-1 beta peptide or an IL-1 beta mutein, wherein said IL-1 beta molecule preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:49 to 62, SEQ ID NO:64, SEQ ID NO:66, SEQ ID NO:89 to 116, SEQ ID NO:130 to SEQ ID NO:140, SEQ ID NO:164, and SEQ ID NO:165. Specifically preferred embodiments of IL-1 beta molecules are human IL-1 beta molecules, preferably human IL-1 beta proteins, human IL-1 beta fragments or human IL-1 beta mature fragments, wherein said IL-1 beta molecules preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:49, SEQ ID NO:64, SEQ ID NO:130 to SEQ ID NO:140 and SEQ ID NO:165, most preferably SEQ ID NO:64.

In a further preferred embodiment said IL-1 molecule is an IL-1 protein, an IL-1 fragment or, preferably, an IL-1 mature fragment, wherein said IL-1 protein, IL-1 fragment or IL-1 mature fragment preferably are capable of binding to the IL-1 receptor and, still more preferably, additionally also comprise biological activity.

In a further preferred embodiment said IL-1 molecule is an IL-1 protein, wherein said IL-1 protein preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:36 to SEQ ID NO:62.

In a further preferred embodiment said IL-1 protein is an IL-1 alpha protein, wherein said IL-1 alpha protein preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:36 to SEQ ID NO:48. Most preferably said IL-1 alpha protein is a human IL-1 alpha protein, wherein said human IL-1 alpha protein preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with SEQ ID NO:36.

In a further preferred embodiment said IL-1 protein is an is an IL-1 beta protein, wherein said IL-1 beta protein preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of the sequences selected from the group consisting of SEQ ID NO:49 to SEQ ID NO:62. Most preferably said IL-1 beta protein is a human IL-1 beta protein, wherein said human IL-1 beta protein preferably comprises or even more preferably consists of a polypeptide, wherein the amino acid sequence of said polypeptide shows least 80%, preferably at least 90%, more preferably at least 95%, even more preferably at least 99% and most preferably 100% sequence identity with SEQ ID NO:49.

In a further preferred embodiment said IL-1 molecule is an IL-1 fragment, preferably an IL-1 mature fragment, and wherein said IL-1 fragment or said IL-1 mature fragment preferably is derived from mouse or human, most preferably human. Preferably said IL-1 fragment or said IL-1 mature fragment comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:63 to SEQ ID NO:66, SEQ ID NO:130, and SEQ ID NO:163 to SEQ ID NO:165.

In a further preferred embodiment said IL-1 mature fragment is an IL-1 alpha mature fragment, wherein said IL-1 alpha mature fragment preferably comprises biological activity and wherein further said IL-1 alpha mature fragment preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:63 or SEQ ID NO:65, most preferably SEQ ID NO:63.

In a further preferred embodiment said IL-1 mature fragment is an IL-1 beta mature fragment, wherein said IL-1 beta mature fragment preferably comprises biological activity and wherein further said IL-1 beta mature fragment preferably comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:64, SEQ ID NO:66, and SEQ ID NO:130, most preferably SEQ ID NO:64.

In a further preferred embodiment said IL-1 molecule is an IL-1 peptide, wherein said IL-1 peptide is derived from mouse, rat or human, most preferably human. Preferably said IL-1 peptide comprises or even more preferably consists of a polypeptide, wherein the amino acid sequence of said polypeptide shows at least 80%, preferably at least 90%, more preferably at least 95%, even more preferably at least 99% and most preferably 100% sequence identity with any one of SEQ ID NO:67 to SEQ ID NO:116.

In a further preferred embodiment said IL-1 molecule is an IL-1 mutein, wherein preferably said IL-1 mutein comprises reduced or more preferably no biological activity, and wherein further preferably said IL-1 mutein is capable of binding the IL-1 receptor. In a further preferred embodiment said IL-1 mutein comprises or preferably consists of a polypeptide, wherein the amino acid sequence of said polypeptide differs from the amino acid sequence of an IL-1 mature fragment in 1 to 3, more preferably in 1 to 2, and most preferably in exactly 1 amino acid residue(s).

In one preferred embodiment, said IL-1 mutein comprises at least one, preferably one, mutated amino acid sequence derived from a wild type amino acid sequence, wherein said wild type amino acid sequence is an IL-1 beta amino acid sequence selected from the group consisting of: (1) position 3 to 11 of SEQ ID NO:64; (2) position 46 to 56 of SEQ ID NO:64; (3) position 88 to 109 of SEQ ID NO:64; and (4) position 143 to 153 of SEQ ID NO:64; or wherein said wild type amino acid sequence is an IL-1 alpha amino acid sequence selected from the group consisting of: (5) position 9 to 20 of SEQ ID NO:63; (6) position 52 to 62 of SEQ ID NO:63; (7) position 94 to 113 of SEQ ID NO:63; and (8) position 143 to 153 of SEQ ID NO:63; and wherein said at least one mutated amino acid sequence is characterized by an amino acid exchange in one to four positions, preferably in one, two or three positions, more preferably in one or two positions, as compared to said wild type amino acid sequence it is derived from; or wherein said at least one mutated amino acid sequence is characterized by a deletion of one to four consecutive amino acids of said wild type amino acid sequence it is derived from.

In a further preferred embodiment said IL-1 mutein comprises at most one mutated amino acid sequence derived from each of said L-1 beta amino acid sequences (1) to (4); or wherein said IL-1 mutein comprises at most one mutated amino acid sequence derived from each of said IL-1 alpha amino acid sequences (5) to (8).

In a very preferred embodiment said IL-1 mutein comprises exactly one of said at least one mutated amino acid sequence, wherein preferably said exactly one mutated amino acid sequence is derived from a wild type amino acid sequence, wherein said wild type amino acid sequence is position 143 to 153 of SEQ ID NO:64 or position 143 to 153 of SEQ ID NO:63.

In a further preferred embodiment said at least one mutated amino acid sequence is characterized by a deletion of one to three, preferably of one to two, consecutive amino acids of said wild type amino acid sequence it is derived from.

In a further preferred embodiment said at least one mutated amino acid sequence is characterized by a deletion of exactly one amino acid of said wild type amino acid sequence it is derived from.

In a further preferred embodiment said at least one mutated amino acid sequence is derived from a wild type amino acid sequence, wherein said wild type amino acid sequence is position 143 to 153 of SEQ ID NO:64 or position 143 to 153 of SEQ ID NO:63. Most preferably said at least one mutated amino acid sequence is derived from position 143 to 153 of SEQ ID NO:64.

In a further preferred embodiment said at least one mutated amino acid sequence is derived from a wild type amino acid sequence, wherein said wild type amino acid sequence is position 46 to 56 of SEQ ID NO:64 or position 52 to 62 of SEQ ID NO:63, wherein preferably said at least one mutated amino acid sequence is characterized by a deletion of one to four, preferably of two to three, consecutive amino acids of said wild type amino acid sequence it is derived from. In a very preferred embodiment said IL-1 mutein comprises or preferably consists of a polypeptide, wherein the amino acid sequence of said polypeptide is SEQ ID NO:137 or SEQ ID NO:138.

In a further preferred embodiment said at least one mutated amino acid sequence is derived from a wild type amino acid sequence, wherein said wild type amino acid sequence is position 88 to 109 of SEQ ID NO:64 or position 94-113 of SEQ ID NO:63, wherein said at least one mutated amino acid sequence is characterized by the deletion of one to four, preferably of one to three, more preferably of one to two consecutive amino acids of said wild type amino acid sequence it is derived from.

In a further preferred embodiment said at least one mutated amino acid sequence is characterized by an amino acid exchange in one or two positions, preferably in exactly one position, as compared to said wild type amino acid sequence it is derived from.

In a further preferred embodiment said wild type amino acid sequence is position 143 to 153 of SEQ ID NO:64 or position 143 to 153 of SEQ ID NO:63 and said at least one mutated amino acid sequence is characterized by an amino acid exchange in one or two positions, preferably in exactly one position, as compared to said wild type amino acid sequence, wherein further preferably said exactly one position is position 145 of SEQ ID NO:64 or position 145 of SEQ ID NO:63, wherein still further preferably said amino acid exchange is an exchange of aspartic acid (D) to an amino acid selected from the group consisting of lysine (K), tyrosine (Y), phenylalanine (F), asparagine (N) and arginine (R).

In a very preferred embodiment said amino acid exchange is an exchange of aspartic acid (D) to lysine (K).

In a further preferred embodiment said wild type amino acid sequence is position 143 to 153 of SEQ ID NO:64 or position 143 to 153 of SEQ ID NO:63 and said at least one mutated amino acid sequence is characterized by an amino acid exchange in exactly one position as compared to said wild type amino acid sequence, wherein further preferably said exactly one position is position 146 of SEQ ID NO:64 or position 146 of SEQ ID NO:63, wherein still further preferably said amino acid exchange is an exchange of phenylalanine (F) to an amino acid selected from the group consisting of asparagine (N), glutamine (Q), and serine (S).

In a further preferred embodiment said IL-1 mutein is an IL-1 beta mutein, preferably a human IL-1 beta mutein, most preferably a human IL-1 beta mutein selected from SEQ ID NO:131 to SEQ ID NO:140, most preferably said IL-1 mutein is SEQ ID NO:136.

In a further preferred embodiment said IL-1 mutein is an IL-1 beta mutein, wherein preferably said IL-1 beta mutein comprises or preferably consists of a polypeptide, wherein the amino acid sequence of said polypeptide differs from the amino acid sequence of SEQ ID NO:64 in 1 to 10, 1 to 9, 1 to 8, 1 to 7, 1 to 6, 1 to 5, 1 to 4, 1 to 3, or 1 to 2 amino acid residues. Most preferably, said amino acid sequence differs from the amino acid sequence of SEQ ID NO:64 in exactly 1 amino acid residue. In a very preferred embodiment, said IL-1 beta mutein comprises or preferably consists of a polypeptide, wherein the amino acid sequence of said polypeptide is selected from SEQ ID NO:131 to SEQ ID NO:140 and SEQ ID NO:205 to SEQ ID NO:209, wherein most preferably said IL-1 beta mutein comprises or preferably consists of a polypeptide, wherein the amino acid sequence of said polypeptide is SEQ ID NO:136.

In a very preferred embodiment, said IL-1 molecule, and preferably said IL-1 beta mutein, comprises or preferably consists of SEQ ID NO:136.

In a further preferred embodiment said IL-1 mutein is an IL-1 alpha mutein, wherein preferably said IL-1 alpha mutein comprises or preferably consists of a polypeptide, wherein the amino acid sequence of said polypeptide differs from the amino acid sequence of SEQ ID NO:63 in 1 to 10, 1 to 9, 1 to 8, 1 to 7, 1 to 6, 1 to 5, 1 to 4, 1 to 3, or 1 to 2 amino acid residues. Most preferably said amino acid sequence differs from the amino acid sequence of SEQ ID NO:63 in exactly 1 amino acid residue. In a very preferred embodiment said IL-1 alpha mutein comprise or preferably consist of a polypeptide, wherein the amino acid sequence of said polypeptide is selected from the group consisting of SEQ ID NO:210 to SEQ ID NO:218, wherein most preferably said IL-1 alpha mutein comprises or preferably consists of a polypeptide, wherein the amino acid sequence of said polypeptide is SEQ ID NO:210.

In a very preferred embodiment, said IL-1 molecule, and preferably said IL-1 alpha mutein comprises or preferably consists of SEQ ID NO:210.

Further disclosed is a method of producing the compositions of the invention comprising (a) providing a VLP with at least one first attachment site; (b) providing at least one antigen with at least one second attachment site, wherein said antigen comprises or consists of an IL-1 molecule, preferably an IL-1 protein, an IL-1 fragment, preferably an IL-1 mature fragment, an IL-1 peptide or an IL-1 mutein; and (c) combining said VLP and said at least one antigen with at least one second attachment site to produce said composition, wherein said at least one antigen and said VLP are linked through the first and the second attachment sites. In a preferred embodiment, the provision of the at least one antigen, comprising said IL-1 molecule, said IL-1 protein, said IL-1 fragment, preferably an IL-1 mature fragment, said IL-1 peptide or said IL-1 mutein, with the at least one second attachment site is by way of expression, preferably by way of expression in a bacterial system, preferably in E. coli. Usually a purification tag, such as His tag, Myc tag, Fc tag or HA tag is added to facilitate the purification process. In another approach particularly the IL-1 peptides or IL-1 muteins with no longer than 50 amino acids are chemically synthesized.

In one preferred embodiment of the invention, the VLP with at least one first attachment site is linked to the antigen with at least one second attachment site via at least one peptide bond. A gene encoding an IL-1 molecule, preferably an IL-1 mutein, is in-frame ligated, either internally or preferably to the N- or the C-terminus to the gene encoding the coat protein of the VLP. Fusion may also be effected by inserting sequences of the IL-1 molecule into a mutant coat protein where part of the coat protein sequence has been deleted. Such constructs are further referred to as truncation mutants. Truncation mutants may have N- or C-terminal, or internal deletions of part of the sequence of the coat protein. For example for the specific VLP HBcAg, amino acids 79-80 are replaced with a foreign epitope. The fusion protein shall preferably retain the ability of assembly into a VLP upon expression which can be examined by electromicroscopy.

Flanking amino acid residues may be added to increase the distance between the coat protein and foreign epitope. Glycine and serine residues are particularly favored amino acids to be used in the flanking sequences. Such a flanking sequence confers additional flexibility, which may diminish the potential destabilizing effect of fusing a foreign sequence into the sequence of a VLP subunit and diminish the interference with the assembly by the presence of the foreign epitope.

In other embodiments, the at least one IL-1 molecule, preferably the IL-1 mutein can be fused to a number of other viral coat proteins, for example to the C-terminus of a truncated form of the A1 protein of Qβ (Kozlovska, T. M., et al., Intervirology 39:9-15 (1996)), or being inserted between position 72 and 73 of the CP extension. As another example, the IL-1 molecule can be inserted between amino acid 2 and 3 of the fr CP, leading to a IL-1-fr CP fusion protein (Pushko P. et al., Prot. Eng. 6:883-891 (1993)). Furthermore, IL-1 can be fused to the N-terminal protuberant β-hairpin of the coat protein of RNA bacteriophage MS-2 (WO 92/13081). Alternatively, the IL-1 can be fused to a capsid protein of papilloma virus, preferably to the major capsid protein L1 of bovine papillomavirus type 1 (BPV-1) (Chackerian, B. et al., Proc. Natl. Acad. Sci. USA 96:2373-2378 (1999), WO 00/23955). Substitution of amino acids 130-136 of BPV-1 L1 with an IL-1 is also an embodiment of the invention. Further embodiments of fusing an IL-1 molecule to coat protein of a virus, or to mutants or fragments thereof, have been disclosed in WO 2004/009124 page 62 line 20 to page 68 line 17 and herein are incorporated by way of reference.

U.S. Pat. No. 5,698,424 describes a modified coat protein of bacteriophage MS-2 capable of forming a capsid, wherein the coat protein is modified by an insertion of a cysteine residue into the N-terminal hairpin region, and by replacement of each of the cysteine residues located external to the N-terminal hairpin region by a non-cysteine amino acid residue. The inserted cysteine may then be linked directly to a desired molecular species to be presented such as an epitope or an antigenic protein.

We note, however, that the presence of an exposed free cysteine residue in the capsid may lead to oligomerization of capsids by way of disulfide bridge formation. Moreover, attachment between capsids and antigenic proteins by way of disulfide bonds are labile, in particular, to sulfhydryl-moiety containing molecules, and are, furthermore, less stable in serum than, for example, thioether attachments (Martin F J. and Papahadjopoulos D. (1982) Irreversible Coupling of Immunoglobulin Fragments to Preformed Vesicles. J. Biol. Chem. 257: 286-288).

Therefore, in a further very preferred embodiment of the present invention, the association or linkage of the VLP and the at least one antigen comprising or consisting of the IL-1 molecule, does not comprise a disulfide bond. In a further preferred embodiment the at least one second attachment comprise, or preferably is, a sulfhydryl group. Moreover, in again a very preferred embodiment of the present invention, the association or linkage of the VLP and the at least one antigen does not comprise a sulphur-sulphur bond. Further preferably, the at least one second attachment comprise, or preferably is, a sulfhydryl group. In a further very preferred embodiment, said at least one first attachment site is not or does not comprise a sulfhydryl group. In again a further very preferred embodiment, said at least one first attachment site is not or does not comprise a sulfhydryl group of a cysteine.

In a further preferred embodiment said at least one first attachment comprises an amino group and said second attachment comprises a sulfhydryl group.

In a further preferred embodiment, said first attachment is an amino group and said second attachment site is a sulfhydryl group. In a still further preferred embodiment, said first attachment is an amino group of a lysine residue, and said second attachment site is a sulfhydryl group of a cysteine residue.

In a further preferred embodiment only one of said second attachment sites associates with said first attachment site through at least one non-peptide covalent bond leading to a single and uniform type of binding of said antigen to said core particle, preferably to said virus-like particle, wherein said only one second attachment site that associates with said first attachment site is a sulfhydryl group, and wherein said antigen and said core particle, preferably said virus-like particle, interact through said association to form an ordered and repetitive antigen array, and wherein further preferably said first attachment site is an amino group of a lysine residue.

In a further preferred embodiment said virus-like particle comprises, essentially consists of, or alternatively consists of, recombinant coat proteins, mutants or fragments thereof, of a virus, preferably of an RNA bacteriophage, wherein said at least one antigen is fused to the N- or the C-terminus of said recombinant coat proteins, mutants or fragments thereof.

In a further preferred embodiment said virus-like particle comprises, essentially consists of, or alternatively consists of, recombinant coat proteins, mutants or fragments thereof, of an RNA bacteriophage, wherein preferably said RNA bacteriophage is selected from the group consisting of: (a) bacteriophage AP205; (b) bacteriophage fr; and (c) bacteriophage GA; and wherein said at least one antigen is fused to the N- or the C-terminus, preferably to the C-terminus, of said recombinant coat proteins, mutants or fragments thereof, and wherein further preferably said at least one antigen comprises or preferably consists of a polypeptide, wherein the amino acid sequence of said polypeptide is SEQ ID NO:136 or SEQ ID NO:210, preferably SEQ ID NO:136.

In a further preferred embodiment an IL-1 molecule, preferably an IL-1 protein, more preferably an IL-1 mature fragment, still more preferably an IL-1 mature fragment comprising or consisting of amino acid sequenced SEQ ID NO:63 to SEQ ID NO:66, most preferably SEQ ID NO:63 or SEQ ID NO:64, is fused to either the N- or the C-terminus, preferably the C-terminus, of a coat protein, mutant or fragments thereof, of RNA bacteriophage AP205.

In a very preferred embodiment said virus-like particle comprises, essentially consists of, or alternatively consists of, recombinant coat proteins, mutants or fragments thereof, of bacteriophage AP205, wherein said at least one antigen is fused to the C-terminus of said recombinant coat proteins, mutants or fragments thereof, and wherein said at least one antigen comprises or preferably consists of a polypeptide, wherein the amino acid sequence of said polypeptide is SEQ ID NO:136 or SEQ ID NO:210, preferably SEQ ID NO:136.

VLPs comprising fusion proteins of coat protein of bacteriophage AP205 with an antigen are generally disclosed in WO2006/032674A1 which is incorporated herein by reference. In one further preferred embodiment, the fusion protein further comprises a linker, wherein said linker is fused to the coat protein, fragments or mutants thereof, of AP205 and the IL-1 molecule. In a further preferred embodiment said IL-1 molecule is fused to the C-terminus of said coat protein, fragments or mutants thereof, of AP205 via said linker.

It has been found that IL-1 molecules, in particular IL-1 proteins and IL-1 fragments comprising at least 100 and up to 300 amino acids, typically and preferably about 140 to 160 amino acids, and most preferably about 155 amino acids, can be fused to coat protein of bacteriophages, preferably to coat protein of AP205, while maintaining the ability of the coat protein to self assemble into a VLP.

Given the large size of IL-1 proteins, IL-1 fragments and IL-1 mature fragments and also for steric reasons, an expression system producing mosaic VLPs comprising AP205 coat proteins fused to an IL-1 molecule as well as wt coat protein subunits was constructed. In this system, suppression of the stop codon yields the AP205-IL-1 coat protein fusion, while proper termination yields the wt AP205 coat protein. Both proteins are produced simultaneously in the cell and assemble into a mosaic VLP. The advantage of such a system is that large proteins can be displayed without interfering with the assembly of the VLP. As the level of incorporation of AP205-IL-1 fusion protein into the mosaic VLP is depending on the level of suppression, AP205-IL-1 is expressed in E. coli cells already containing a plasmid overexpressing a suppressor t-RNA. For opal suppression, plasmid pISM3001 (Smiley, B. K., Minion, F. C. (1993) Enhanced readthrough of opal (UGA) stop codons and production of Mycoplasma pneumoniae P1 epitopes in Escherichia coli. Gene 134, 33-40), which encodes a suppressor t-RNA recognizing the opal stop codon and introducing Trp is used. Suppression of amber termination can be increased by use of plasmid pISM579, which overexpresses a suppressor t-RNA recognizing the amber stop codon and introducing Trp as well. Plasmid pISM579 was generated by excising the trpT176 gene from pISM3001 with restriction endonuclease EcoRI and replacing it by an EcoRI fragment from plasmid pMY579 (gift of Michael Yarus) containing an amber t-RNA suppressor gene. This t-RNA suppressor gene is a mutant of trpT175 (Raftery L A. Et al. (1984) J. Bacteriol. 158:849-859), and differs from trpT at three positions: G33, A24 and T35. Expression of the AP205-interleukin-1alpha fusion protein in an E. coli strain with amber suppression (supE or glnV) such as E. coli JM109 may generate a proportion of AP205-IL-1 fusion proteins with a Gln instead of Trp introduced at the amber stop codon, in addition to AP205-IL-1 fusion proteins with a Trp introduced at the amber stop codon. The identity of the amino acid translated at the stop codon may therefore depend on the combination of suppressor t-RNA overexpressed, and strain phenotype. As described by Miller J H et al. ((1983) J. Mol. Biol. 164: 59-71) and as is well known in the art, the efficiency of suppression is context dependent. In particular, the codon 3′ of the stop codon and the first base 3′ from the stop codon are particularly important. For example, stop codons followed by a purine base are in general well suppressed.

Thus, in a preferred embodiment said VLP is a mosaic VLP, wherein said mosaic VLP comprises or preferably consists of at least one, preferably one, first polypeptide and of at least one, preferably one, second polypeptide, wherein said first polypeptide is a recombinant capsid protein, mutant or fragments thereof; and wherein said second polypeptide is a genetic fusion product of a recombinant capsid protein, mutant or fragments thereof, preferably of said first polypeptide, with an IL-1 molecule. In a further preferred embodiment said first polypeptide is a recombinant capsid protein of bacteriophage AP205 or a mutant or fragment thereof. In a further preferred embodiment said first polypeptide is selected from SEQ ID NO:21, SEQ ID NO:22, SEQ ID NO:23. In a very preferred embodiment said first polypeptide is SEQ ID NO:21. Mosaic VLPs of bacteriophage AP205 comprising an antigen are generally disclosed in WO2006/032674A1, in particular in paragraph 107 of said publication. In a further preferred embodiment said second polypeptide is a genetic fusion product of a recombinant capsid protein, mutant or fragments thereof, preferably of said first polypeptide, with an IL-1 molecule, wherein said IL-1 molecule is fused to the C-terminus of said recombinant capsid protein, mutant or fragments thereof, preferably via an amino acid linker. In a further preferred embodiment said IL-1 molecule comprises or preferably consists of 100 to 300 amino acids, typically and preferably about 140 to 160 amino acids, and most preferably about 155 amino acids. In a very preferred embodiment, the molar ratio of said first polypeptide and said second polypeptide in said mosaic VLP is 10:1 to 5:1, preferably 8:1 to 6:1, most preferably about 7:1.

In one preferred embodiment of the present invention, the composition comprises or alternatively consists essentially of a virus-like particle with at least one first attachment site linked to at least one antigen with at least one second attachment site via at least one covalent bond, wherein preferably the covalent bond is a non-peptide bond. In a preferred embodiment of the present invention, the first attachment site comprises, or preferably is, an amino group, preferably the amino group of a lysine residue. In another preferred embodiment of the present invention, the second attachment site comprises, or preferably is, a sulfhydryl group, preferably a sulfhydryl group of a cysteine.

In a very preferred embodiment of the invention, the at least one first attachment site is an amino group, preferably an amino group of a lysine residue and the at least one second attachment site is a sulfhydryl group, preferably a sulfhydryl group of a cysteine.

In one preferred embodiment of the invention, the antigen is linked to the VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, i.e. a sulfhydryl group, preferably of cysteine(s) residue inherent of, or artificially added to the IL-1 molecule, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulthydryl groups. Most preferably, said hetero-bifunctional cross-linker is succinimidyl-6-[β-maleimidopropionamido]hexanoate (SMPH). Another class of cross-linkers suitable in the practice of the invention is characterized by the introduction of a disulfide linkage between the IL-1 molecule and the VLP upon coupling. Preferred cross-linkers belonging to this class include, for example, SPDP and Sulfo-LC-SPDP (Pierce).

In a preferred embodiment, the composition of the invention further comprises a linker. In a further preferred embodiment said at least one antigen with said at least one second attachment site further comprises a linker, wherein said linker comprises said second attachment site, and wherein said linker is associated to said antigen by way of one peptide bond, and wherein preferably said linker is a cysteine. Engineering of a second attachment site onto the IL-1 molecule is achieved by the association of a linker, preferably containing at least one amino acid residue suitable as second attachment site according to the disclosures of this invention. Therefore, in a preferred embodiment of the present invention, a linker is associated to the IL-1 molecule by way of at least one covalent bond, preferably, by at least one, preferably one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue.

The selection of a linker will be dependent on the nature of the IL-1 molecule, on its biochemical properties, such as pI, charge distribution and glycosylation. In general, flexible amino acid linkers are favored. In a further preferred embodiment of the present invention, the linker consists of amino acids, wherein further preferably the linker consists of at least one and at most 25, preferably at most 20, more preferably at most 15 amino acids. In an again preferred embodiment of the invention, the amino acid linker contains 1 to 10 amino acids. Preferred embodiments of the linker are selected from the group consisting of: (a) CGG (SEQ ID NO:171); (b) N-terminal gamma 1-linker, preferably CGDKTHTSPP (SEQ ID NO:172); (c) N-terminal gamma 3-linker, preferably CGGPKPSTPPGSSGGAP (SEQ ID NO:173); (d) Ig hinge regions; (e) N-terminal glycine linkers, preferably GCGGGG (SEQ ID NO:174); (f) (G)kC(G)n with n=0-12 and k=0-5 (SEQ ID NO:175); (g) N-terminal glycine-serine linkers, preferably (GGGGS)n, n=1-3 (SEQ ID NO:176) with one further cysteine; (h) (G)kC(G)m(S)l(GGGGS)n with n=0-3, k=0-5, m=0-10, l=0-2 (SEQ ID NO:177); (i) GGC (SEQ ID NO:178); (k) GGC-NH2 (SEQ ID NO:179); (l) C-terminal gamma 1-linker, preferably DKTHTSPPCG (SEQ ID NO:180); (m) C-terminal gamma 3-linker, preferably PKPSTPPGSSGGAPGGCG (SEQ ID NO:181); (n) C-terminal glycine linkers, preferably GGGGCG (SEQ ID NO:182)); (o) (G)nC(G)k with n=0-12 and k=0-5 (SEQ ID NO:183); (p) C-terminal glycine-serine linkers, preferably (SGGGG)n n=1-3 (SEQ ID NO:184) with one further cysteine; (q) (G)m(S)l(GGGGS)n(G)oC(G)ₖ with n=0-3, k=0-5, m=0-10, l=0-2, and o=0-8 (SEQ ID NO:185). In a further preferred embodiment the linker is added to the N-terminus of the IL-1 molecule. In another preferred embodiment of the invention, the linker is added to the C-terminus of IL-1 molecule.

Preferred linkers according to this invention are glycine linkers (G)n further containing a cysteine residue as second attachment site, such as N-terminal glycine linker (GCGGGG, SEQ ID NO:174) and C-terminal glycine linker (GGGGCG, SEQ ID NO:182). Further preferred embodiments are C-terminal glycine-lysine linker (GGKKGC, SEQ ID NO:186) and N-terminal glycine-lysine linker (CGKKGG, SEQ ID NO:187), GGCG (SEQ ID NO:188) and GGC (SEQ ID NO:178) or GGC-NH2 (SEQ ID NO:179, “NH2” stands for amidation) linkers at the C-terminus of the peptide or CGG (SEQ ID NO:171) at its N-terminus. In general, glycine residues will be inserted between bulky amino acids and the cysteine to be used as second attachment site, to avoid potential steric hindrance of the bulkier amino acid in the coupling reaction. In a further preferred embodiment said linker further comprises a His-tag. A very preferred linker comprises or preferably consists of LEHHHHHHGGC (SEQ ID NO:201) or LEHHHHHHGGCG (SEQ ID NO:219), preferably the linker consists of LEHHHHHHGGCG (SEQ ID NO:219).

Linking of the antigen with at least one second attachment site to the VLP by using a hetero-bifunctional cross-linker according to the preferred methods described above, allows coupling of the IL-1 molecule to the VLP in an oriented fashion. Other methods of linking the antigen with at least one second attachment site to the VLP include methods wherein the IL-1 molecule is cross-linked to the VLP, using the carbodiimide EDC, and NHS. The IL-1 molecule may also be first thiolated through reaction, for example with SATA, SATP or iminothiolane. The antigen with at least one second attachment site after deprotection if required, may then be coupled to the VLP as follows. After separation of the excess thiolation reagent, the antigen with at least one second attachment site is reacted with the VLP, previously activated with a hetero-bifunctional cross-linker comprising a cysteine reactive moiety, and therefore displaying at least one or several functional groups reactive towards cysteine residues, to which the thiolated antigen with at least one second attachment site can react, such as described above. Optionally, low amounts of a reducing agent are included in the reaction mixture. In further methods, the antigen with at least one second attachment site is attached to the VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the VLP.

In other embodiments of the present invention, the composition comprises or alternatively consists essentially of a virus-like particle linked to antigen with at least one second attachment site via chemical interactions, wherein at least one of these interactions is not a covalent bond.

Linking of the VLP to the antigen with at least one second attachment site can be effected by biotinylating the VLP and expressing the IL-1 molecule as a streptavidin-fusion protein.

One or several antigen molecules, i.e. IL-1 molecules, can be attached to one subunit of the VLP, preferably of RNA bacteriophage coat proteins, preferably through the exposed lysine residues of the coat proteins of RNA bacteriophage VLP, if sterically allowable. A specific feature of the VLPs of RNA bacteriophage and in particular of the Qβ coat protein VLP is thus the possibility to couple several antigens per subunit. This allows for the generation of a dense antigen array.

In very preferred embodiments of the invention, the antigen with at least one second attachment site is linked via a cysteine residue, having been added to either the N-terminus or the C-terminus of, or a natural cysteine residue within an IL-1 molecule, to lysine residues of coat proteins of the VLPs of RNA bacteriophage, and in particular to the coat protein of Qβ.

As described above, four lysine residues are exposed on the surface of the VLP of Qβ coat protein. Typically and preferably these residues are derivatized upon reaction with a cross-linker molecule. In the instance where not all of the exposed lysine residues can be coupled to an antigen, the lysine residues which have reacted with the cross-linker are left with a cross-linker molecule attached to the E-amino group after the derivatization step. This leads to disappearance of one or several positive charges, which may be detrimental to the solubility and stability of the VLP. By replacing some of the lysine residues with arginines, as in the disclosed Qβ coat protein mutants, we prevent the excessive disappearance of positive charges since the arginine residues do not react with the preferred cross-linkers. Moreover, replacement of lysine residues by arginine residues may lead to more defined antigen arrays, as fewer sites are available for reaction to the antigen.

Accordingly, exposed lysine residues were replaced by arginines in the following Qβ coat protein mutants: Qβ-240 (Lys13-Arg; SEQ ID NO:16), Qβ-250 (Lys 2-Arg, Lys13-Arg; SEQ ID NO:18), Qβ-259 (Lys 2-Arg, Lys16-Arg; SEQ ID NO:20) and Qβ-251; (Lys16-Arg, SEQ ID NO:19). In a further embodiment, we disclose a Qβ mutant coat protein with one additional lysine residue Qβ-243 (Asn 10-Lys; SEQ ID NO:17), suitable for obtaining even higher density arrays of antigens.

In one preferred embodiment of the invention, the VLP of an RNA bacteriophage is recombinantly produced by a host and wherein said VLP is essentially free of host RNA, preferably host nucleic acids. In one further preferred embodiment, the composition further comprises at least one polyanionic macromolecule bound to, preferably packaged in or enclosed in, the VLP. In a still further preferred embodiment, the polyanionic macromolecule is polyglutamic acid and/or polyaspartic acid.

In another preferred embodiment, the composition further comprises at least one immunostimulatory substance bound to, preferably packaged in or enclosed in, the VLP. In a still further preferred embodiment, the immunostimulatory substance is a nucleic acid, preferably DNA, most preferably an unmethylated CpG containing oligonucleotide.

Essentially free of host RNA, preferably host nucleic acids: The term “essentially free of host RNA, preferably host nucleic acids” as used herein, refers to the amount of host RNA, preferably host nucleic acids, comprised by the VLP, which amount typically and preferably is less than 30 μg, preferably less than 20 μg, more preferably less than 10 μg, even more preferably less than 8 μg, even more preferably less than 6 μg, even more preferably less than 4 μg, most preferably less than 2 μg, per mg of the VLP. Host, as used within the afore-mentioned context, refers to the host in which the VLP is recombinantly produced. Conventional methods of determining the amount of RNA, preferably nucleic acids, are known to the skilled person in the art. The typical and preferred method to determine the amount of RNA, preferably nucleic acids, in accordance with the present invention is described in Example 17 of WO2006/037787A2. Identical, similar or analogous conditions are, typically and preferably, used for the determination of the amount of RNA, preferably nucleic acids, for inventive compositions comprising VLPs other than Qβ. The modifications of the conditions eventually needed are within the knowledge of the skilled person in the art. The numeric value of the amounts determined should typically and preferably be understood as comprising values having a deviation of ±10%, preferably having a deviation off ±5%, of the indicated numeric value.

Host RNA, preferably host nucleic acids: The term “host RNA, preferably host nucleic acids” or the term “host RNA, preferably host nucleic acids, with secondary structure”, as used herein, refers to the RNA, or preferably nucleic acids, that are originally synthesized by the host. The RNA, preferably nucleic acids, may, however, undergo chemical and/or physical changes during the procedure of reducing or eliminating the amount of RNA, preferably nucleic acids, typically and preferably by way of the inventive methods, for example, the size of the RNA, preferably nucleic acids, may be shortened or the secondary structure thereof may be altered. However, even such resulting RNA or nucleic acids is still considered as host RNA, or host nucleic acids.

Methods to determine the amount of RNA and to reduce the amount of RNA comprised by the VLP have disclosed in US provisional application filed by the same assignee on Oct. 5, 2004 and thus the entire application is incorporated herein by way of reference. Reducing or eliminating the amount of host RNA, preferably host nucleic, minimizes or reduces unwanted T cell responses, such as inflammatory T cell response and cytotoxic T cell response, and other unwanted side effects, such as fever, while maintaining strong antibody response specifically against IL-1.

In one preferred embodiment, this invention provides a method of preparing the inventive compositions and VLP of an RNA bacteriophage the invention, wherein said VLP is recombinantly produced by a host and wherein said VLP is essentially free of host RNA, preferably host nucleic acids, comprising the steps of: a) recombinantly producing a virus-like particle (VLP) with at least one first attachment site by a host, wherein said VLP comprises coat proteins, variants or fragments thereof, of a RNA bacteriophage; b) disassembling said virus-like particle to said coat proteins, variants or fragments thereof, of said RNA bacteriophage; c) purifying said coat proteins, variants or fragments thereof; d) reassembling said purified coat proteins, variants or fragments thereof, of said RNA bacteriophage to a virus-like particle, wherein said virus-like particle is essentially free of host RNA, preferably host nucleic acids; and e) linking at least one antigen of the invention with at least one second attachment site to said VLP obtained from step d). In a further preferred embodiment, the reassembling of said purified coat proteins, variants or fragments thereof, is effected in the presence of at least one polyanionic macromolecule.

In one aspect, the invention provides a vaccine for the treatment, amelioration and/or prevention of diabetes, preferably of type II diabetes, said vaccine comprising the composition of the invention, preferably in an effective amount. Thus, the invention provides a vaccine for the treatment, amelioration and/or prevention of diabetes, preferably of type II diabetes, said vaccine comprising a composition, preferably in an effective amount, comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site.

An effective amount of a composition of the invention is an amount which is capable of inducing an immune response in the treated subject, preferably in a human, and which preferably results in a therapeutic or prophylactic effect in diabetes, preferably in type II diabetes.

In a preferred embodiment, said vaccine comprises (i) a first composition, preferably in an effective amount, wherein said first composition is a composition according to the invention wherein the IL-1 molecule comprised by said first composition is an IL-1 beta molecule, preferably SEQ ID NO:136 or SEQ ID NO:165; and (ii) a second composition, preferably in an effective amount, wherein said second composition is a composition according to the invention wherein the IL-1 molecule comprised by said second composition is an IL-1 alpha molecule, preferably SEQ ID NO:203 or SEQ ID NO:210.

In one preferred embodiment, the IL-1 molecule which is linked to the VLP in the composition contained in the vaccine may be of animal, preferably mammal or human origin. In preferred embodiments, the IL-1 of the invention is of human, bovine, dog, cat, mouse, rat, pig or horse origin.

In one embodiment, the vaccine further comprises at least one adjuvant.

An advantageous feature of the present invention is the high immunogenicity of the composition, even in the absence of adjuvants. Therefore, in a preferred embodiment, the vaccine composition is devoid of adjuvant. The absence of an adjuvant, furthermore, minimizes the occurrence of unwanted inflammatory T-cell responses representing a safety concern in the vaccination against self antigens. Thus, the administration of the vaccine of the invention to a patient will preferably occur without administering at least one adjuvant to the same patient prior to, simultaneously or after the administration of the vaccine.

However, when an adjuvant is administered, the administration of the at least one adjuvant may hereby occur prior to, simultaneously or after the administration of the inventive composition or of the vaccine.

When the composition and/or the vaccine of the invention is administered to an individual, it may be in a form which contains salts, buffers, adjuvants, or other substances which are desirable for improving the efficacy of the conjugate. Examples of materials suitable for use in preparation of vaccines or pharmaceutical compositions are provided in numerous sources including Remington's Pharmaceutical Sciences (Osol, A, ed., Mack Publishing Co., (1990)). This includes sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

The vaccines of the invention are said to be “pharmaceutically acceptable” if their administration can be tolerated by a recipient individual, preferably by a human. Further, the vaccines of the invention are administered in a “therapeutically effective amount” (i.e., an amount that produces a desired physiological effect). The nature or type of immune response is not a limiting factor of this disclosure. Without the intention to limit the present invention by the following mechanistic explanation, the inventive vaccine might induce antibodies which bind to IL-1 and thus reduce its concentration and/or interfering with its physiological or pathological function.

The invention therefore provides a pharmaceutical composition for the treatment, amelioration and/or prevention of diabetes, preferably of type II diabetes, said pharmaceutical composition comprising (1) a composition comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site; and (2) a pharmaceutically acceptable carrier.

The invention further provides a pharmaceutical composition for the treatment, amelioration and/or prevention of diabetes, preferably of type II diabetes, said pharmaceutical composition comprising (1) a vaccine, said vaccine comprising a composition comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site; and (2) a pharmaceutically acceptable carrier.

The invention further provides a method for the treatment, amelioration and/or prevention of diabetes, preferably of type II diabetes, said method comprising administering a composition, a vaccine or a pharmaceutical composition of the invention to an animal, preferably to a human. The invention further provides a method for the treatment, amelioration and/or prevention of diabetes, preferably of type II diabetes, said method comprising administering to an animal, preferably to a human, (i) a first composition, preferably in an effective amount, wherein said first composition is a composition according to the invention wherein the IL-1 molecule comprised by said first composition is an IL-1 beta molecule, preferably SEQ ID NO:136 or SEQ ID NO:165; and (ii) a second composition, preferably in an effective amount, wherein said second composition is a composition according to the invention wherein the IL-1 molecule comprised by said second composition is an IL-1 alpha molecule, preferably SEQ ID NO:203 or SEQ ID NO:210.

Thus, the invention provides a method for the treatment, amelioration and/or prevention of diabetes, preferably of type II diabetes, said method comprising administering a composition to an animal, preferably to a human, said comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site.

The invention further provides a method for the treatment, amelioration and/or prevention of diabetes, preferably of type II diabetes, said method comprising administering a vaccine to an animal, preferably to a human, said vaccine comprising a composition comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site.

The invention further provides a method for the treatment, amelioration and/or prevention of diabetes, preferably of type II diabetes, said method comprising administering a pharmaceutical composition to an animal, preferably to a human, said pharmaceutical composition comprising (1) a composition comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site; and (2) a pharmaceutically acceptable carrier.

The invention further provides a method for the treatment, amelioration and/or prevention of diabetes, preferably of type II diabetes, said method comprising administering a pharmaceutical composition to an animal, preferably to a human, said pharmaceutical composition comprising (1) a vaccine, said vaccine comprising a composition comprising (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site; and (2) a pharmaceutically acceptable carrier.

With respect to the methods of the invention, said composition, said vaccine and/or said pharmaceutical composition is administered to said animal, preferably to said human, in an immunologically effective amount.

In further preferred embodiments said animal is a mammal, preferably selected from cat, sheep, pig, horse, cattle, dog, rat, mouse, and, most preferably, human.

In one embodiment, the compositions, vaccines and/or pharmaceutical compositions are administered to said animal, preferably to said human by injection, infusion, inhalation, oral administration, or other suitable physical methods. In a preferred embodiment, the compositions, vaccines and/or pharmaceutical compositions are administered to said animal, preferably to said human, intramuscularly, intravenously, transmucosally, transdermally, intranasally, intraperitoneally, subcutaneously, or directly into the lymph node.

A further aspect of the invention is the use of the compositions, the vaccines and/or of the pharmaceutical compositions described herein for the treatment, amelioration and/or prevention of diabetes, preferably of type II diabetes. In more detail, the invention provides for the use of a composition for the treatment, amelioration and/or prevention of diabetes, preferably of type II diabetes, said composition comprising: (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site.

A further aspect of the invention is the use of the compositions, the vaccines and/or of the pharmaceutical compositions described herein for the manufacture of a medicament for the treatment, amelioration and/or prevention of diabetes, preferably of type II diabetes. In more detail, the invention provides for the use of a composition for the manufacture of a medicament for the treatment, amelioration and/or prevention of diabetes, preferably of type II diabetes, said composition comprising: (a) a virus-like particle (VLP) with at least one first attachment site; and (b) at least one antigen with at least one second attachment site; wherein said at least one antigen comprises or consists of an IL-1 molecule and wherein (a) and (b) are linked through said at least one first and said at least one second attachment site.

It is to be understood that all technical features and embodiments described herein, in particular those described for the compositions of the invention, may be applied to all aspects of the invention, especially to the vaccine, pharmaceutical compositions, methods and uses, alone or in any possible combination. In this context it is explicitly underlined that the following embodiments of said at least one antigen with at least one second attachment site are specifically preferred.

In a further preferred embodiment said at least one antigen with at least one second attachment site comprises or preferably consists of (i) an IL-1 beta molecule, wherein said IL-1 beta molecule is selected from any one of SEQ ID NO:165, and SEQ ID NOs 131 to 140; and (ii) a linker, wherein said linker comprises said second attachment site, and wherein preferably said linker comprises or preferably consists of GGC (SEQ ID NO:178) or GGCG (SEQ ID NO:188).

In a further preferred embodiment said at least one antigen with at least one second attachment site consists of (i) an IL-1 beta molecule, wherein said IL-1 beta molecule is SEQ ID NO:165 or SEQ ID NO:136, preferably SEQ ID NO:136; and (ii) a linker, wherein said linker comprises said second attachment site, and wherein said linker comprises or preferably consists of GGC (SEQ ID NO:178) or GGCG (SEQ ID NO:188), preferably GGCG (SEQ ID NO:188).

In a further preferred embodiment said at least one antigen with at least one second attachment site consists of (i) an IL-1 beta molecule, wherein said IL-1 beta molecule is SEQ ID NO:165 or SEQ ID NO:136, preferably SEQ ID NO:136; and (ii) a linker, wherein said linker comprises said second attachment site, and wherein said linker is covalently bound to the C-terminus of said IL-1 beta molecule by way of a peptide bond, and wherein said linker comprises or preferably consists of GGC (SEQ ID NO:178) or GGCG (SEQ ID NO:188), preferably of GGCG (SEQ ID NO:188).

In a further preferred embodiment said at least one antigen with at least one second attachment site consists of (i) an IL-1 beta molecule, wherein said IL-1 beta molecule is SEQ ID NO:165 or SEQ ID NO:136, preferably SEQ ID NO:136; and (ii) a linker, wherein said linker comprises said second attachment site, and wherein said linker is covalently bound to the C-terminus of said IL-1 beta molecule by way of a peptide bond, and wherein said linker consists of LEHHHHHHGGCG (SEQ ID NO:219).

In a further preferred embodiment said at least one antigen with at least one second attachment site is any one of SEQ ID NOs 220 to 223, preferably SEQ ID NO:220.

In a further preferred embodiment said at least one antigen with at least one second attachment site comprises or preferably consists of (i) an IL-1 alpha molecule, wherein said IL-1 alpha molecule is selected from any one of SEQ ID NOs 203 to 218; and (ii) a linker, wherein said linker comprises said second attachment site, and wherein preferably said linker comprises or preferably consists of GGC (SEQ ID NO:178) or GGCG (SEQ ID NO:188).

In a further preferred embodiment said at least one antigen with at least one second attachment site consists of (i) an IL-1 alpha molecule, wherein said IL-1 alpha molecule is SEQ ID NO:203 or SEQ ID NO:210, preferably SEQ ID NO:203; and (ii) a linker, wherein said linker comprises said second attachment site, and wherein said linker comprises or preferably consists of GGC (SEQ ID NO:178) or GGCG (SEQ ID NO:188), preferably GGCG (SEQ ID NO:188).

In a further preferred embodiment said at least one antigen with at least one second attachment site consists of (i) an IL-1 alpha molecule, wherein said IL-1 alpha molecule is SEQ ID NO:203 or SEQ ID NO:210, preferably SEQ ID NO:203; and (ii) a linker, wherein said linker comprises said second attachment site, and wherein said linker is covalently bound to the C-terminus of said IL-1 alpha molecule by way of a peptide bond, and wherein said linker comprises or preferably consists of GGC (SEQ ID NO:178) or GGCG (SEQ ID NO:188), preferably of GGCG (SEQ ID NO:188).

In a further preferred embodiment said at least one antigen with at least one second attachment site is any one of SEQ ID NOs 224 or 225, preferably SEQ ID NO:224.

### Example 1

### Cloning, Expression and Purification of Murine IL1α₁₁₇₋₂₇₀ and IL-1β₁₁₉₋₂₆₉

The nucleotide sequence encoding amino acids 117-270 of murine IL-1α was amplified by PCR from a cDNA library of TNFα-activated murine macrophages using oligonucleotides IL1α1 (5′-ATATATGCTAGCCCCTTACACCTACCAGAGTGATTTG-3′; SEQ ID NO:24) and IL1α2 (5′-ATATATCTCGAGTGATATCTGGAAGTCTGTCATA GAG-3′; SEQ ID NO:25). Using the same cDNA library, the nucleotide sequence encoding amino acids 119-269 of the murine IL-113 precursor was amplified with oligonucleotides IL1β1 (5′-ATATATGCTAGCCCCCATTAGACAGCTGCACTACAGG-3′; SEQ ID NO:26) and IL1β2 (5′-ATATATCTCGAGGGAAGACACAGATTCCATGGTGAAG-3′; SEQ ID NO: 27). Both DNA fragments were digested with NheI and XhoI, and cloned into the expression vector pModEC1 (SEQ ID NO:29)

The vector pModEC1 (SEQ ID NO:29) is a derivative of pET22b(+) (Novagen Inc.), and was constructed in two steps. In a first step the multiple cloning site of pET22b(+) was changed by replacing the original sequence between the NdeI and XhoI sites with the annealed oligos primerMCS-1F (5′-TATGGATCCGGCTAGCGCTCGAGGGTTTA AACGGCGGCCGCAT-3′; SEQ ID NO:30) and primerMCS-1R (5′-TCGAATGCGGCCG CCGTTTAAACCCTCGAGCGCTAGCCGGATCCA-3′; SEQ ID NO:31) (annealing in 15 mM TrisHCl pH 8 buffer). The resulting plasmid was termed pMod00, and had NdeI, BamHI, NheI, XhoI, PmeI and NotI restriction sites in its multiple cloning site. The annealed pair of oligos Bamhis6-EK-Nhe-F (5′-GATCCACACCACCACCACCACCACGG TTCTGGTGACGACGATGACAAAGCGCTAGCCC-3′; SEQ ID NO:32) and Bamhis6-EKNhe-R (5′-TCGAGGGCTAGCGCTTTGTCATCGTCGTCACCAGAACCGTGGT GGTGGTGGTGGTGTG-3′; SEQ ID NO:33) and the annealed pair of oligo1F-C-glycine-linker (5′-TCGAGGGTGGTGGTGGTGGTTGCGGTTAATAAGTTTAAACGC-3′; SEQ ID NO:34) and oligo1R-C-glycine-linker (5′-GGCCGCGTTTAAACTTATTA ACCGCAACCACCACCACCACCC-3′; SEQ ID NO:35) were ligated together into the BamHI-NotI digested pMod00 plasmid to obtain pModEC1, which encodes an N-terminal hexahistidine tag, an enterokinase cleavage site and a C-terminal glycine linker containing one cysteine residue.

The cloning of the above mentioned fragments into pModEC1 gave rise to plasmids pModEC1-His-EK-mIL1α₁₁₇₋₂₇₀ and pModEC1-His-EK-mIL1β₁₁₉₋₂₆₉, respectively. These plasmids encode fusion proteins consisting of an N-terminal His-tag, an enterokinase cleavage site, the mature murine IL-1α or IL-1β, respectively, and a C-terminal cysteine-containing linker (GGGGGCG, SEQ ID NO:28). For expression, Escherichia coli BL21 cells harbouring either plasmid were grown at 37° C. to an OD at 600 nm of 1.0 and then induced by addition of isopropyl-β-D-thiogalactopyranoside at a concentration of 1 mM. Bacteria were grown for 4 more hours at 37° C., harvested by centrifugation and resuspended in 80 ml lysis buffer (10 mM Na₂HPO₄, 30 mM NaCl, pH 7.0). Cells were then disrupted by sonication and cellular DNA and RNA were digested by 30 min incubation at room temperature with 64 μM MgCl₂ and 10 μl Benzonase. Cellular debris was removed by centrifugation (SS34 rotor, 20000 rpm, 4° C., 60 min), and the cleared lysate was applied to a Ni²⁺-NTA agarose column (Qiagen, Hilden, Germany). After extensive washing of the column with washing buffer (50 mM NaH₂PO₄, 300 mM NaCl, 20 mM Imidazol, pH 8.0) the proteins were eluted with elution buffer (50 mM NaH₂PO₄, 300 mM NaCl, 200 mM Imidazol, pH 8.0). Purified proteins were dialysed against PBS pH 7.2, flash-frozen in liquid nitrogen and stored at −80° C. until further use.

### Example 2

### A. Coupling of Mouse IL-1β119-269 to Qβ Virus-Like Particles

A solution containing 1.3 mg/ml of the purified murine IL-1β₁₁₉₋₂₆₉ protein from EXAMPLE 1 (SEQ ID NO:66) in PBS pH 7.2 was incubated for 60 min at room temperature with an equimolar amount of TCEP for reduction of the C-terminal cysteine residue.

A solution of 6 ml of 2 mg/ml Qβ capsid protein in PBS pH 7.2 was then reacted for 60 min at room temperature with 131 μl of a SMPH solution (65 mM in DMSO). The reaction solution was dialysed at 4° C. against three 3 l changes of 20 mM HEPES, 150 mM NaCl pH 7.2 over 24 hours. Seventy-five μl of the derivatized and dialyzed Qβ solution was mixed with 117 μl H₂O and 308 μl of the purified and pre-reduced mouse IL-1β₁₁₉₋₂₆₉ protein and incubated over night at 15° C. for chemical crosslinking. Uncoupled protein was removed by tangential flow filtration against PBS using cellulose ester membranes with a molecular weight cutoff of 300.000 Da.

Coupled products were analyzed on a 12% SDS-polyacrylamide gel under reducing conditions. The Coomassie stained gel is shown in FIG. 1. Several bands of increased molecular weight with respect to the Qβ capsid monomer are visible, clearly demonstrating the successful cross-linking of the mouse IL-1β₁₁₉₋₂₆₉ protein to the Qβ capsid.

### B. Immunization of Mice with Mouse IL-1β₁₁₉₋₂₆₉ Protein Coupled to Qβ Capsid (Qβ-mIL-1β₁₁₉₋₂₆₉)

Five female balb/c mice were immunized with Qβ-mIL-1β₁₁₉₋₂₆₉ (SEQ ID NO:66). Fifty μg of total protein were diluted in PBS to 200 μl and injected subcutaneously (100 μl on two ventral sides) on day 0 and day 21. Mice were bled retroorbitally on day 0, 21, and 35, and sera were analyzed using mouse IL-1β₁₁₉₋₂₆₉-specific ELISA.

### C. ELISA

ELISA plates were coated with mouse IL-1β₁₁₉₋₂₆₉ protein at a concentration of 1 μg/ml. The plates were blocked and then incubated with serially diluted mouse sera from day 0, 21, and 35. Bound antibodies were detected with enzymatically labeled anti-mouse IgG antibody. Antibody titers of mouse sera were calculated as the average of those dilutions which led to half maximal optical density at 450 nm. The average anti-mouse IL-1β₁₁₉₋₂₆₉ titer was 1:22262 at day 21 and 1:309276 at day 35. This demonstrates that immunization with coupled to the mouse IL-1β₁₁₉₋₂₆₉ protein could overcome immunological tolerance and produce high titer antibodies which recognize specifically IL-1β₁₁₉₋₂₆₉.

### D. In Vitro Neutralization of IL-1β

Sera of mice immunized with Qβ-mIL-1β₁₁₉₋₂₆₉ (SEQ ID NO:66) were then tested for their ability to inhibit the binding of mouse IL-1β protein to its receptor. ELISA plates were therefore coated with a recombinant mIL-1receptorI-hFc fusion protein at a concentration of 1 μg/ml, and co-incubated with serial dilutions of sera from mice which had been immunized either with mouse IL-1β₁₁₉₋₂₆₉ coupled to Qβ capsid or with mouse IL-1α₁₁₇₋₂₇₀ coupled to Qβ capsid and 100 ng/ml of mouse IL-1β₁₁₉₋₂₆₉. Binding of IL-1β₁₁₉₋₂₆₉ to the immobilized mIL-1receptorI-hFc fusion protein was detected with a biotinylated anti-mouse IL-1β antibody and horse radish peroxidase conjugated streptavidin. All sera from mice immunized against murine IL-1β₁₁₉₋₂₆₉ inhibited completely the binding of mouse IL-1β₁₁₉₋₂₆₉ to its receptor at concentrations of ≧0.4%, whereas sera from mice immunized against mouse IL-1α₁₁₇₋₂₇₀ did not show any inhibitory effect even at the highest concentration used (3.3%). These data demonstrate that immunization with mouse IL-1β₁₁₉₋₂₆₉ coupled to Qβ capsid can yield antibodies which are able to neutralize the interaction of mouse IL-1β₁₁₉₋₂₆₉ and its receptor.

### E. In Vivo Neutralization of IL-1β

The in vivo neutralizing capacity of the antibodies raised by immunization with Qβ-mIL-1β₁₁₉₋₂₆₉ was investigated next. Four female balb/c mice were therefore immunized twice at days 0 and 14 with Qβ-mIL-1β₁₁₉₋₂₆₉ and four mice were immunized at the same time with Qβ capsid alone. At day 21 all mice were injected intravenously with 1 μg free IL-1β₁₁₉₋₂₆₉. As readout of the inflammatory activity of the injected IL-1β₁₁₉₋₂₆₉, serum samples were analysed 3 h after injection for the relative increase in the concentration of the pro-inflammatory cytokine IL-6. Qβ-immunized mice showed an average increase in the serum IL-6 concentration of 1.01±0.61 ng/ml, whereas mice immunized with Qβ-mIL-1β₁₁₉₋₂₆₉ showed an average increase of only 0.11±0.30 ng/ml (p=0.04). As a control on day 28 all mice were injected with 1 μg mIL-1α. Three hours after injection mice immunized with Qβ carrier alone showed an average increase in serum IL-6 concentrations of 40.24±8.06 ng/ml, while mice immunized with Qβ-mIL-1β₁₁₉₋₂₆₉ showed an increase of 57.98±29.92 ng/ml (p=0.30). These data indicate that the antibodies produced by immunization with Qβ-mIL-1β₁₁₉₋₂₆₉ were able to neutralize specifically and efficiently the pro-inflammatory activity of IL-1β.

### Example 3

### A. Coupling of Mouse IL-1α₁₁₇₋₂₇₀ to QβVirus-Like Particles

A solution containing 1.8 mg/ml of the purified IL-1α₁₁₇₋₂₇₀ protein from EXAMPLE 1 (SEQ ID NO:65) in PBS pH 7.2 was incubated for 60 min at room temperature with an equimolar amount of TCEP for reduction of the C-terminal cysteine residue.

A solution of 6 ml of 2 mg/ml Qβ capsid protein in PBS pH 7.2 was then reacted for 60 minutes at room temperature with 131 μl of a SMPH solution (65 mM in DMSO). The reaction solution was dialyzed at 4° C. against three 3 l changes of 20 mM HEPES, 150 mM NaCl pH 7.2 over 24 hours. Seventy-five μl of the derivatized and dialyzed Qβ solution was mixed with 192 μl H₂O and 233 μl of the purified and pre-reduced mouse IL-1α₁₁₇₋₂₇₀ protein and incubated over night at 15° C. for chemical crosslinking. Uncoupled protein was removed by tangential flow filtration against PBS using cellulose ester membranes with a molecular weight cutoff of 300.000 Da.

Coupled products were analyzed on a 12% SDS-polyacrylamide gel under reducing conditions. The Coomassie stained gel is shown in FIG. 2. Several bands of increased molecular weight with respect to the Qβ capsid monomer are visible, clearly demonstrating the successful cross-linking of the mouse IL-1α₁₁₇₋₂₇₀ protein to the Qβ capsid.

### B. Immunization of Mice with Mouse IL-1α₁₁₇₋₂₇₀ Protein Coupled to Qβ Capsid (Qβ-mIL-1α₁₁₇₋₂₇₀)

Five female balb/c mice were immunized with Qβ-mIL-1α₁₁₇₋₂₇₀. Fifty μg of total protein were diluted in PBS to 200 μl and injected subcutaneously (100 μl on two ventral sides) on day 0 and day 21. Mice were bled retroorbitally on day 0, 21, and 35, and sera were analyzed using mouse IL-1α₁₁₇₋₂₇₀-specific ELISA.

### C. ELISA

ELISA plates were coated with mouse IL-1α₁₁₇₋₂₇₀ protein at a concentration of 1 μg/ml. The plates were blocked and then incubated with serially diluted mouse sera from day 0, 21, and 35. Bound antibodies were detected with enzymatically labeled anti-mouse IgG antibody. Antibody titers of mouse sera were calculated as the average of those dilutions which led to half maximal optical density at 450 nm. The average anti-mouse IL-1α₁₁₇₋₂₇₀ titer was 1:9252 at day 21 and 1:736912 at day 35. This demonstrates that immunization with coupled to the mouse IL-1α₁₁₇₋₂₇₀ protein could overcome immunological tolerance and produce high titer antibodies which recognize specifically IL-1α₁₁₇₋₂₇₀.

### D. In Vitro Neutralization of IL-1α

Sera of mice immunized with Qβ-mIL-1α₁₁₇₋₂₇₀ were then tested for their ability to inhibit the binding of mouse IL-1α protein to its receptor. ELISA plates were therefore coated with a recombinant mIL-1receptorI-hFc fusion protein at a concentration of 1 μg/ml, and co-incubated with serial dilutions of sera from mice which had been immunized either with mouse IL-1α₁₁₇₋₂₇₀ coupled to Qβ capsid or with mouse IL-1β₁₁₉₋₂₆₉ coupled to Qβ capsid and 5 ng/ml of mouse IL-1α₁₁₇₋₂₇₀. Binding of IL-1α₁₁₇₋₂₇₀ to the immobilized mIL-1receptorI-hFc fusion protein was detected with a biotinylated anti-mouse IL-1α antibody and horse radish peroxidase conjugated streptavidin. All sera from mice immunized against murine IL-1α₁₁₇₋₂₇₀ inhibited completely the binding of mouse IL-1α₁₁₇₋₂₇₀ to its receptor at concentrations of ≧0.4%, whereas sera from mice immunized against mouse IL-1β₁₁₉₋₂₆₉ did not show a significant inhibitory effect even at the highest concentration used (3.3%). These data demonstrate that immunization with mouse IL-1α₁₁₇₋₂₇₀ coupled to Qβ capsid can yield antibodies which are able to neutralize specifically the interaction of mouse IL-1α₁₁₇₋₂₇₀ and its receptor.

### E. In Vivo Neutralization of IL-1α

The in vivo neutralizing capacity of the antibodies raised by immunization with Qβ-mIL-1α₁₁₇₋₂₇₀ was investigated next. Four female balb/c mice were therefore immunized twice at days 0 and 14 with Qβ-mIL-1α₁₁₇₋₂₇₀ and four mice were immunized at the same time with Qβ capsid alone. At day 21 all mice were injected intravenously with 1 μg free IL-1α₁₁₇₋₂₇₀. As readout of the inflammatory activity of the injected IL-1α₁₁₇₋₂₇₀, serum samples were analysed 3 h after injection for the relative increase in the concentration of the pro-inflammatory cytokine IL-6. Qβ-immunized mice showed an average increase in the serum IL-6 concentration of 8.16±2.33 ng/ml, whereas mice immunized with Qβ-mIL-1α₁₁₇₋₂₇₀ showed an average increase of only 0.15±0.27 ng/ml (p=0.0005). As a control on day 28 all mice were injected with 1 μg mIL-1β. Three hours after injection mice immunized with Qβ carrier alone showed an average increase in serum IL-6 concentrations of 9.52±7.33 ng/ml, while mice immunized with Qβ-mIL-1α₁₁₇₋₂₇₀ showed an increase of 21.46±27.36 ng/ml (p=0.43). These data indicate that the antibodies produced by immunization with Qβ-mIL-1α₁₁₇₋₂₇₀ were able to neutralize specifically and efficiently the pro-inflammatory activity of IL-1α.

### Example 4

### Comparison of Qβ-mIL-1α₁₁₇₋₂₇₀ and Qβ-mIL-1β₁₁₉₋₂₆₉ Immunization to Kineret® Treatment in a Mouse Model of Rheumatoid Arthritis

Kineret® (Anakinra, Amgen) is a recombinant version of the human IL-1 receptor antagonist, which is approved for the treatment of human rheumatoid arthritis. In order to reach a clinical benefit, relatively high amounts (100 mg) have to be applied via subcutaneous injection on a daily basis. The collagen-induced arthritis model was used to compare the efficacy of Qβ-mIL-1α₁₁₇₋₂₇₀ and Qβ-mIL-1β₁₁₉₋₂₆₉ immunization with daily applications of different doses of Kineret®. Male DBA/1 mice were immunized subcutaneously three times (days 0, 14 and 28) with 50 μg of either Qβ-mIL-1α₁₁₇₋₂₇₀ (n=8), Qβ-mIL-1β₁₁₉₋₂₆₉ (n=8) or Qβ alone (n=32), and then injected intradermally on day 42 with 200 μg bovine type II collagen mixed with complete Freund's adjuvant. From day 42 on, mice immunized with Qβ-mIL-1α₁₁₇₋₂₇₀ and Qβ-mIL-1β₁₁₉₋₂₆₉, and one group of Qβ-immunized mice (n=8) received daily intraperitoneal injections of 200 μl PBS, while three additional Qβ-immunized groups received daily intraperitoneal injections of either 37.5 μg (n=8), 375 μg (n=8), or 3.75 mg (n=8) Kineret®. A daily injection of 37.5 μg Kineret® per mouse corresponds roughly to a dose of 1.5 mg/kg, which is in the range of the recommended efficacious amount for humans (100 mg). All mice were boosted on day 63 by intradermal injection of 200 μg bovine type II collagen mixed with incomplete Freund's adjuvant, and examined on a daily basis for the development of arthritis symptoms.

Four weeks after the second collagen injection, Qβ-immunized control mice showed an average cumulative clinical score (as defined in EXAMPLE 2F of WO2008/037504A1) of 3.75, while Qβ-mIL-1α₁₁₇₋₂₇₀- and Qβ-mIL-1β₁₁₉₋₂₆₉-immunized mice showed average scores of only 0.81 and 1.44, respectively (see Table 1). Mice treated with 37.5 μg or 375 μg Kineret® reached an average score of 2.44 and 2.63, respectively, while mice treated with 3.75 mg Kineret® remained largely asymptomatic, reaching a maximal score of only 0.19.

As an additional readout of the inflammatory reaction, the hind ankle thickness of all animals was measured on a regular basis. Four weeks after the second collagen injection Qβ-immunized control mice showed an average increase in hind ankle thickness of 16%, while Qβ-mIL-1α₁₁₇₋₂₇₀-immunized mice showed an increase of 2% and Qβ-mIL-1β₁₁₉₋₂₆₉-immunized mice showed an increase of 6%. Mice treated with either 37.5 μg or 375 μg Kineret® showed an average increase of 13% and 10%, respectively, while mice treated with 3.75 mg Kineret® showed no increase in hind ankle thickness at all.

In conclusion we surprisingly found that three injections of either Qβ-mIL-1α₁₁₇₋₂₇₀ or Qβ-mIL-1β₁₁₉₋₂₆₉ protected mice better from the development of arthritis symptoms than daily injections of Kineret® in amounts corresponding to the human dose or even the ten-fold human dose. Only application of the 100-fold human dose of Kineret® showed an increased benefit with respect to Qβ-mIL-1α₁₁₇₋₂₇₀ or Qβ-mIL-1β₁₁₉₋₂₆₉ vaccination.

TABLE 1
clinical disease symptoms in collagen-induced arthritis model.

| | | Average increase in |
|---|---|---|
| | average clinical | hind ankle thickness |
| Treatment | score day 91 | (%) day 63-91 |
| 3x Qβ s.c. + PBS i.p. (200 μl/day) | 3.75 | 16 |
| 3x Qβ-mIL-1α₁₁₇₋₂₇₀ s.c. + | 0.81 | 2 |
| PBS i.p. (200 μl/day) | | |
| 3x Qβ-mIL-1β₁₁₉₋₂₆₉ s.c. + | 1.44 | 6 |
| PBS i.p. (200 μl/day) | | |
| 3x Qβ s.c. + Kineret ® i.p. | 2.44 | 13 |
| (37.5 μg/day) | | |
| 3x Qβ s.c. + Kineret ® i.p. | 2.63 | 10 |
| (375 μg/day) | | |
| 3x Qβ s.c. + Kineret ® i.p. | 0.19 | 0 |
| (3.75 mg/day) | | |

### Example 5

### A. Cloning, Expression, and Purification of Virus-Like Particles Consisting of AP205 Coat Protein Genetically Fused to Mouse IL-1α₁₁₇₋₂₇₀ (AP205_mILα₁₁₇₋₂₇₀)

Given the large size of interleukin-1alpha and for steric reasons, an expression system producing so called mosaic particles, comprising AP205 coat proteins fused to interleukin-1alpha as well as wt coat protein subunits was constructed. In this system, suppression of the stop codon yields the AP205-interleukin-1alpha coat protein fusion, while proper termination yields the wt AP205 coat protein. Both proteins are produced simultaneously in the cell and assemble into a mosaic virus-like particle. Two intermediary plasmids, pAP590 and pAP592, encoding the AP205 coat protein gene terminated by the suppressor codons TAG (amber, pAP590) or TGA (opal, pAP592) were made. A linker sequence encoding the tripeptide Gly-Ser-Gly (SEQ ID NO:189) was added downstream and in frame of the coat protein gene. Kpn2I and HindIII sites were added for cloning sequences encoding foreign amino acid sequences at the C-terminus of the Gly-Ser-Gly amino acid linker, C-terminal to the AP205 coat protein. The resulting constructs were: AP590 (SEQ ID NO:117): AP205 coat protein gene—amber codon—GSG(Kpn2I-HindIII); and AP592 (SEQ ID NO:118): AP205 coat protein gene—opal codon—GSG(Kpn2I-HindIII). For construction of plasmid pAP590, a PCR fragment obtained with oligonucleotides p1.44 (5′-NNCCATGGCAAATAAGCCAATGCAACCG-3′; SEQ ID NO:119) and pINC-36 (5′-GTAAGCTTAGATGCATTATCCGGA TCCCTAAGCAGTAGTATCAGACGATACG-3′; SEQ ID NO:120) was digested with NcoI and HindIII, and cloned into vector pQb185, which had been digested with the same restriction enzymes. pQb185 is a vector derived from pGEM vector. Expression of the cloned genes in this vector is controlled by the trp promoter (Kozlovska, T. M. et al., Gene 137:133-37 (1993)). Similarly, plasmid pAP592 was constructed by cloning a NcoI/HindIII-digested PCR fragment obtained with oligonucleotides p1.44 and pINC-40 (5′-GTAAGCTTAGATGCATTATCCGGATCCTCAAGCAGTAGTA TCAGACGATACG-3′; SEQ ID NO:121) into the same vector.

The sequence encoding amino acids 117-270 of murine IL-1α was amplified by PCR from plasmid pModEC1-His-EK-mIL1α₁₁₇₋₂₇₀ (see EXAMPLE 1), using primers pINC-34 (5′-GGTCCGGAGCGCTAGCCCCTTACAC-3′; SEQ ID NO:122) and pINC-35 (5′-GTAAGCTTATGCATTATGATATCTGGAAGTCTGTCATAGA-3′; SEQ ID NO:123), which added Kpn2I and HindIII restriction sites to the 5′ and 3′ ends, respectively. The obtained DNA fragment was digested with Kpn2I and HindIII and cloned into both vector pAP590, creating plasmid pAP594 (amber suppression), and into vector pAP592, creating plasmid pAP596 (opal suppression), respectively.

For expression of mosaic AP205 VLPs displaying murine IL-1α on their surface, E. coli JM109 cells containing plasmid pISM 579 or pISM 3001 were transformed with plasmid pAP594 or pAP596, respectively. Plasmid pISM579 was generated by excising the trpT176 gene from pISM3001 with restriction endonuclease EcoRI and replacing it by an EcoRI fragment from plasmid pMY579 (gift of Michael Yarus) containing an amber t-RNA suppressor gene. This t-RNA suppressor gene is a mutant of trpT175 (Raftery L A. Et al. (1984) J. Bacteriol. 158:849-859), and differs from trpT at three positions: G33, A24 and T35. Five milliliters of LB liquid medium containing 20 μg/ml ampicillin and 10 μg/ml Kanamycin were inoculated with a single colony, and incubated at 37° C. for 16-24 h without shaking. The prepared inoculum was diluted 50× with M9 medium containing 20 μg/ml ampicillin and 10 μg/ml Kanamycin and incubated at 37° C. overnight on a shaker. Cells were harvested by centrifugation.

Cells (1 g, transformed with plasmid pAP594 and containing pISM579) were lysed by ultrasonication in lysis buffer (20 mM Tris-HCl, 5 mM EDTA, 150 mM NaCl, pH 7.8, 0.1% Tween 20). The lysate was cleared by centrifugation, and the cell debris were washed with lysis buffer. Pooled supernatant were loaded on a Sepharose CL-4B column eluted in TEN buffer (20 mM Tris-HCl, 5 mM EDTA, 150 mM NaCl, pH 7.8). The presence of capsids in the cleared lysate and wash supernatant was confirmed by agarose gel electrophoresis (1% TAE, ethidium bromide stained gel and UV detection). Two peaks eluted from the column as determined by SDS-PAGE or UV-spectrometric analysis of light scattering at 310 nm Fractions of the second peak, containing the capsids, were pooled and loaded on a Sepharose CL-6B column. Peak fractions from the CL-6B column were pooled and concentrated using a centrifugal filter unit (Amicon Ultra 15 MWCO 30000, Millipore). The protein was purified further by one additional round of gel filtration on a CL-4B column, and the resulting peak fractions were pooled and concentrated on a centrifugal filter unit as above. The buffer was exchanged to 10 mM Hepes, pH 7.5, and glycerol was added to a final concentration of 50%.

Purification of AP205_mILα₁₁₇₋₂₇₀ from plasmid pAP596 was performed essentially as described for pAP594 above, with the inclusion of an additional sucrose gradient purification step after the last CL-4B column. The protein was layered on a gradient prepared with the following sucrose solutions: 9 ml 36%, 3 ml 30%, 6 ml 25%, 8 ml 20%, 6 ml 15%, 6 ml 10% and 3 ml 5% sucrose. Fractions were identified by UV spectroscopy, and pooled fractions containing the capsids were concentrated on a centrifugal filter unit as above, and the buffer exchanged to 10 mM Hepes, pH 7.5. Glycerol was finally added to a final concentration of 50%.

### B. Immunization of Mice with AP205_mILα₁₁₇₋₂₇₀

Four female balb/c mice were immunized with AP205_-mIL-1α₁₁₇₋₂₇₀. Twentyfive μg of total protein were diluted in PBS to 200 μl and injected subcutaneously (100 μl on two ventral sides) on day 0, day 14, and day 28. Mice were bled retroorbitally on days 0, 14, 28 and 35, and sera were analyzed using mouse IL-1α₁₁₇₋₂₇₀-specific ELISA.

### C. ELISA

ELISA plates were coated with mouse IL-1α₁₁₇₋₂₇₀ protein at a concentration of 1 μg/ml. The plates were blocked and then incubated with serially diluted mouse sera from days 14, 28 and 35. Bound antibodies were detected with enzymatically labeled anti-mouse IgG antibody. Antibody titers of mouse sera were calculated as the average of those dilutions which led to half maximal optical density at 450 nm. The average anti-mouse IL-1α₁₁₇₋₂₇₀ titer was 1:4412 at day 14, 1:27955 on day 28 and 1:34824 on day 35. This demonstrates that immunization with AP205_mIL-1α₁₁₇₋₂₇₀ could overcome immunological tolerance and produce high titer antibodies which recognize specifically IL-1α₁₁₇₋₂₇₀.

### D. In Vitro Neutralization of IL-1α

Sera of mice immunized with AP205_mIL-1α₁₁₇₋₂₇₀ were tested for their ability to inhibit the binding of mouse IL-1α protein to its receptor. ELISA plates were therefore coated with a recombinant mIL-1receptorI-hFc fusion protein at a concentration of 1 μg/ml, and co-incubated with serial dilutions of sera from mice which had been immunized either with AP205_mIL-1α₁₁₇₋₂₇₀ or with AP205 alone and 100 ng/ml of mouse IL-1α₁₁₇₋₂₇₀. Binding of mIL-1α₁₁₇₋₂₇₀ to the immobilized mIL-1receptorI-hFc fusion protein was detected with a biotinylated anti-mouse IL-1α antibody and horse radish peroxidase conjugated streptavidin. All sera from mice immunized AP205_mIL-1α₁₁₇₋₂₇₀ inhibited completely the binding of mouse IL-1α₁₁₇₋₂₇₀ to its receptor at concentrations of ≧3.3%, whereas sera from mice immunized with AP205 did not show a significant inhibitory effect at any concentration used. These data demonstrate that immunization with AP205_mILα₁₁₇₋₂₇₀ can yield antibodies which are able to neutralize specifically the interaction of mouse IL-1α₁₁₇₋₂₇₀ with its receptor.

### E. In Vivo Neutralization of IL-1α

The in vivo neutralizing capacity of the antibodies raised by immunization with AP205_mIL-1α₁₁₇₋₂₇₀ was investigated next. Four female balb/c mice were therefore immunized three times on days 0, 14, and 28 with AP205_mILα₁₁₇₋₂₇₀ and four mice were immunized at the same time with AP205 alone. On day 42 all mice were injected intravenously with 1 μg of free murine IL-1α₁₁₇₋₂₇₀. As readout of the inflammatory activity of the injected IL-1α₁₁₇₋₂₇₀, serum samples were withdrawn before and 3 h after injection and analyzed for the relative increase in the concentration of the pro-inflammatory cytokine IL-6. AP205-immunized mice showed an average increase in the serum IL-6 concentration of 12.92±3.95 ng/ml, whereas mice immunized with AP205_mIL-1α₁₁₇₋₂₇₀ showed an average increase of only 0.06±0.05 ng/ml (p<0.01). These data indicate that the antibodies produced by immunization with AP205_mILα₁₁₇₋₂₇₀ were able to neutralize specifically and efficiently the pro-inflammatory activity of IL-1α.

### Example 6

### A. Cloning and Expression of Virus-Like Particles Consisting of AP205 Coat Protein Genetically Fused to Mouse IL-1β₁₁₉₋₂₆₉ (AP205_mIL-1β₁₁₉₋₂₆₉)

Cloning, expression and purification of virus-like particles consisting of AP205 coat protein genetically fused to mouse IL-1β₁₁₉₋₂₆₉ is carried out essentially as described for AP205_mIL-1α₁₁₇₋₂₇₀ in EXAMPLE 5. The sequence of murine interleukin 1 beta was amplified from plasmid pModEC1-His-EK-mIL1β₁₁₉₋₂₆₉ coding for murine interleukin 1 beta using primers pINC-75 (5′-GATCCGGAGGTGGTGTCCCCATTAGACAGCT-3′, SEQ ID NO:192) and pINC-77 (5′-GTAAGCTTAGGAAGACACAGATTCCAT-3′, SEQ ID NO:193). These primers amplify a murine interleukin-1 beta gene with 5′ Kpn2I and 3′ Hind III sites, and encoding additionally the amino acid sequence Gly-Gly at the N-terminus of murine interleukin 1beta. The obtained mur-IL-1β fragment was digested with Kpn2I and HindIII and cloned in the same restriction sites into vector pAP590 (amber suppression) creating plasmid pAP630. E. coli JM109 containing plasmid pISM 579, providing amber suppression, was transformed with plasmid pAP630. 5 ml of LB liquid medium with 20 μg/ml ampicillin and 10 μg/ml kanamycin were inoculated with a single colony, and incubated at 37° C. for 16-24 h without shaking. The prepared inoculum was diluted 50× with M9 medium containing 20 μg/ml ampicillin and 10 μg/ml kanamycin and incubated at 37° C. overnight on a shaker. Cells were harvested by centrifugation.

### B. Cloning and Expression of Virus-Like Particles Consisting of AP205 Coat Protein Genetically Fused to Human IL-1β₁₁₆₋₂₆₉ (AP205_hIL-1β₁₁₆₋₂₆₉)

The sequence of human interleukin 1 beta was amplified from plasmid pET42T-hIL-1β₁₁₆₋₂₆₉ coding for human interleukin 1 beta using primers pINC-74 (5′-GA TCC GGA GGT GGT GCC CCT GTA CGA TCA CTG AAC TG-3′, SEQ ID NO:194) and pINC-76 (5′-GTATGCATTAGGAAGACACAAATTGCATGGTGAAGTC-3, SEQ ID NO:195), introducing a 5′ Kpn2I and 3′ Mph1103I site, respectively. The obtained human-IL-1β fragment was digested with Kpn2I and Mph1103I and cloned in the same restriction sites into vector pAP590 (amber suppression) creating plasmid pAP649. E. coli JM109 containing plasmid pISM 579 (providing amber suppression), was transformed with plasmid pAP649. 5 ml of LB liquid medium with 20 μg/ml ampicillin and 10 μg/ml canamicin were inoculated with a single colony, and incubated at 37° C. for 16-24 h without shaking. The prepared inoculum was diluted 50× with M9 medium containing 20 μg/ml ampicillin and 10 μg/ml kanamycin and incubated at 37° C. overnight on a shaker. Cells were harvested by centrifugation.

### C. Immunization of Mice with AP205_mIL-1β₁₁₉₋₂₆₉

Four female C3H/HeJ mice were immunized with AP205_mIL-1β₁₁₉₋₂₆₉. Twentyfive μg of total protein were diluted in PBS to 200 μl and injected subcutaneously (100 μl on two ventral sides) on day 0, day 14, and day 28. Mice were bled retroorbitally on days 0, 14, 28 and 35, and sera were analyzed using mIL-1β₁₁₉₋₂₆₉-specific ELISA.

### D. ELISA

ELISA plates were coated with mouse IL-1β₁₁₉₋₂₆₉ protein at a concentration of 1 μg/ml. The plates were blocked and then incubated with serially diluted mouse sera from days 0, 14, 28, and 35. Bound antibodies were detected with enzymatically labeled anti-mouse IgG antibody. Antibody titers of mouse sera were calculated as the average of those dilutions which lead to half maximal optical density at 450 nm. The average anti-mouse IL-1β₁₁₉₋₂₆₉ titer was 1:19000 on day 14, 1:58200 on day 28 and 1:104700 on day 35. This demonstrates that immunization with AP205_mIL-1β₁₁₉₋₂₆₉ could overcome immunological tolerance and produce high titer antibodies which recognize specifically mouse IL-1β₁₁₉₋₂₆₉.

### E. In Vitro Neutralization of IL-1β

Sera of mice immunized with AP205_mIL-1β₁₁₉₋₂₆₉ are then tested for their ability to inhibit the binding of mouse IL-1β protein to its receptor. ELISA plates are therefore coated with a recombinant mIL-1receptorI-hFc fusion protein at a concentration of 1 μg/ml, and co-incubated with serial dilutions of sera from mice immunized either with AP205_mIL-1β₁₁₉₋₂₆₉ or with AP205 alone, and 100 ng/ml of mouse IL-1β₁₁₉₋₂₆₉. Binding of IL-1β₁₁₉₋₂₆₉ to the immobilized mIL-1receptorI-hFc fusion protein is detected with a biotinylated anti-mouse IL-113 antibody and horse radish peroxidase conjugated streptavidin. All sera from mice immunized with AP205_mIL-1β₁₁₉₋₂₆₉ strongly inhibit the binding of mouse IL-1β₁₁₉₋₂₆₉ to its receptor, whereas sera from mice immunized with AP205 alone do not show any inhibitory effect. These data demonstrate that immunization with AP205_mIL-1β₁₁₉₋₂₆₉ can yield antibodies which are able to neutralize the interaction of mouse IL-1β₁₁₉₋₂₆₉ and its receptor.

### F. In Vivo Neutralization of IL-1β

The in vivo neutralizing capacity of the antibodies raised by immunization with AP205_mIL-1β₁₁₉₋₂₆₉ were investigated next. Four female C3H/HeJ mice were therefore immunized three times on days 0, 14, and 28 with AP205_mIL-1β₁₁₉₋₂₆₉ and four mice were immunized at the same time with AP205 alone. On day 42 all mice were injected intravenously with 1 μg of free mIL-1β₁₁₉₋₂₆₉. As readout of the inflammatory activity of the injected mIL-1β₁₁₉₋₂₆₉, serum samples were withdrawn before and 3 h after injection and analysed for the relative increase in the concentration of the pro-inflammatory cytokine IL-6. AP205-immunized mice showed an increase of 0.28 ng/ml in serum IL-6 concentrations, whereas mice immunized with AP205_mIL-1β₁₁₉₋₂₆₉ showed no increase at all. These data indicate that the antibodies produced by immunization with AP205_mIL-1β₁₁₉₋₂₆₉ were able to neutralize specifically and efficiently the pro-inflammatory activity of IL-1β.

### H. Immunization of Mice with AP205_mIL-1β₁₁₆₋₂₆₉

Four female C3H/HeJ mice were immunized with AP205_mIL-1β₁₁₆₋₂₆₉. Twentyfive μg of total protein were diluted in PBS to 200 μl and injected subcutaneously (100 μl on two ventral sides) on days 0, 14, and 28. Mice were bled retroorbitally on days 0, 14, 28 and 35, and sera were analyzed using human IL-1β₁₁₆₋₂₆₉-specific ELISA.

### I. ELISA

ELISA plates were coated with human IL-1β₁₁₆₋₂₆₉ protein at a concentration of 1 μg/ml. The plates were blocked and then incubated with serially diluted mouse sera from days 0, 14, 28, and 35. Bound antibodies were detected with enzymatically labeled anti-mouse IgG antibody. Antibody titers of mouse sera were calculated as the average of those dilutions which lead to half maximal optical density at 450 nm. The average anti-human IL-1β₁₁₆₋₂₆₉ titer was 1:39600 on day 14, 1:58300 on day 28 and 1:65600 on day 35. This demonstrates that AP205_hIL-1β₁₁₆₋₂₆₉ induces high titers of hIL-1β₁₁₆₋₂₆₉-specific antibodies in mice.

### Example 7

### A. Cloning, Expression and Purification of Human IL-1β₁₁₆₋₂₆₉

The nucleotide sequence encoding amino acids 116-269 of human IL-1β (hIL-1β₁₁₆₋₂₆₉) was amplified by PCR from a cDNA library of human liver tissue using oligonucleotides HIL-1 (5′-ATATATGATATCCCTGTACGATCACTGAACTGCACG-3′; SEQ ID NO:124) and HIL-2 (5′-ATATATCTCGAGGGAAGACA CAAATTGCATGGTGAAG-3′; SEQ ID NO:125), digested with XhoI and EcoRV and cloned into the expression vector pET42T(+).

Plasmid pET-42T(+) was constructed by replacing the whole region between the T7 promoter and the T7 terminator of pET-42a(+) (Novagen) in two steps by new linker sequences, which facilitate the expression of a protein of interest as a fusion with a C-terminal tag (SEQ ID NO:190) comprising a His-tag and a cysteine containing linker. In a first step plasmid pET-42a(+) was digested with the restriction enzymes NdeI and AvrII, liberating a 958 bp fragment between the T7 promoter and T7 terminator composed of a GST-tag, S-tag, two His-tags and the multiple cloning site. The residual 4972 bp fragment containing the vector backbone of pET-42a(+) was isolated and ligated to the annealed complementary oligonucleotides 42-1 (5′-TATGGATATCGAATTCAAGCTTCTGCAGCTGCTCGAGTAA TTGATTAC-3′; SEQ ID NO:126) and 42-2 (5′-CTAGGTAATC AATTACTCGA GCAGCTGCAGAAGCTTGAATTCGATATCCA-3′; SEQ ID NO:127), giving rise to plasmid pET-42S(+). In the second step plasmid pET-42S(+) was linearized by digestion with restriction enzymes XhoI and AvrII, and ligated to the complementary annealed oligonucleotides 42T-1 (5′-TCGAGCACCACCACCACCACCACGGTGGTT GCTAATAATAATTGATTAATAC-3′; SEQ ID NO:128) and 42T-2 (5′-CTAGGTATTAATCAATTATTATTAGCAACCACCGTGGTGGTGGTGGTGGTGC-3′; SEQ ID NO:129), resulting in plasmid pET-42T(+).

The cloning of the above mentioned fragment hIL-1β₁₁₆₋₂₆₉ into pET-42T(+) gave rise to plasmid pET42T-hIL-1β₁₁₆₋₂₆₉. This plasmid encodes a fusion protein corresponding to the mature human IL-1β and a His-tag and a C-terminal cysteine-containing linker (GGC, SEQ ID NO:178). Thus, the fusion protein consists of SEQ ID NO:190 C-terminally fused to SEQ ID NO:165. The original alanine residue at position 117 of human IL-1β was changed to isoleucin in this fusion protein. Expression and purification of the human IL-1β₁₁₆₋₂₆₉ protein was performed essentially as described for the murine mIL1β₁₁₉₋₂₆₉ protein in EXAMPLE 1.

### B Cloning, Expression and Purification of Human IL-1β₁₁₆₋₂₆₉ Muteins

By site directed mutagenesis of the plasmid pET42T-hIL-1β₁₁₆₋₂₆₉, expression vectors for ten different mutant human IL-1β₁₁₆₋₂₆₉ fusion proteins were constructed. To this aim the Quik-Change® Site directed mutagenesis kit (Stratagene) was used according to the manufacturer's instructions. The expression vectors for these mutant IL-1β₁₁₉₋₂₆₉ proteins are listed in Table 2 together with the oligonucleotide pairs used for their construction. Expression and purification of the different human IL-1β₁₁₆₋₂₆₉ muteins was performed as described in EXAMPLE 1.

TABLE 2
Overview over IL-1 muteins, expression vectors and oligonucleotides used
for their construction.

| | mutein sequence | |
|---|---|---|
| | (without | |
| Expression vector | purification tag) | Oligonucleotide pair |
| pET42T-hIL-1β₁₁₆₋₂₆₉ | hIL-1β₁₁₆₋₂₆₉(R4D) | R4D-1 (5′-CATATGGATA TCCCTGTAGA |
| (R4D) | (SEQ ID NO: 131) | CTCACTGAAC TGCACGCTC-3′; SEQ ID NO: 143); |
| | | R4D-2 (5′-GAGCGTGCAG TTCAGTGAGT |
| | | CTACAGGGAT ATCCATATG-3′; SEQ ID NO: 144) |
| pET42T-hIL-1β₁₁₆₋₂₆₉ | hIL-1β₁₁₆₋₂₆₉(L6A) | L6A-1 (5′-GATATCCCTG TACGATCAGC |
| (L6A) | (SEQ ID NO: 132) | TAACTGCACG CTCCGGGAC-3′; SEQ ID NO: 145); |
| | | L6A-2 (5′-GTCCCGGAGC GTGCAGTTAG |
| | | CTGATCGTAC AGGGATATC-3′; SEQ ID NO: 146) |
| pET42T-hIL-1β₁₁₆₋₂₆₉ | hIL-1β₁₁₆₋₂₆₉(T9G) | T9G-1 (5′-GTACGATCAC TGAACTGCGG |
| (T9G) | (SEQ ID NO: 133) | TCTCCGGGAC TCACAGC-3′; SEQ ID NO: 147) |
| | | T9G-2 (5′-GCTGTGAGTC CCGGAGACCG |
| | | CAGTTCAGTG ATCGTAC-3′; SEQ ID NO: 148) |
| pET42T-hIL-1β₁₁₆₋₂₆₉ | hIL-1β₁₁₆₋₂₆₉(R11G) | R11G-1 (5′-GAACTGCACG CTCGGGGACT CACAGC-3′; |
| (R11G) | (SEQ ID NO: 134) | SEQ ID NO: 149) |
| | | R11G-2 (5′-GCTGTGAGTC CCCGAGCGTG CAGTTC-3′; |
| | | SEQ ID NO: 150) |
| pET42T-hIL-1β₁₁₆₋₂₆₉ | hIL-1β₁₁₆₋₂₆₉(D54R) | D54R-1 (5′-CAAGGAGAAGAAAGTAATCGCAAAATACCTG |
| (D54R) | (SEQ ID NO: 135) | TGGCCTTG-3′; SEQ ID NO: 151 |
| | | D54R-2 (5′-CAAGGCCACAGGTATTTTGCGATTACTTTCT |
| | | TCTCCTTG-3′; SEQ ID NO: 152) |
| pET42T-hIL-1β₁₁₆₋₂₆₉ | hIL-1β₁₁₆₋₂₆₉(D145K) | D145K-1 (5′-GCGGCCAGGATATAACTAAATTCACCATGC |
| (D145K) | (SEQ ID NO: 136) | AATTTGTGTC-3′; SEQ ID NO: 161) |
| | | D145K-2 (5′-GACACAAATTGCATGGTGAATTTAGTTATA |
| | | TCCTGGCCGC-3′; SEQ ID NO: 162) |
| pET42T-hIL-1β₁₁₆₋₂₆₉ | hIL-1β₁₁₆₋₂₆₉ | EE-1 (5′-CATGTCCTTTGTACAAGGAAGTAATGACAAAAT |
| (ΔEE^{50, 51}) | (ΔEE^{50, 51}) | ACCTGTG-3′; SEQ ID NO: 153) |
| | (SEQ ID NO: 137) | EE-2 (5′-CACAGGTATTTTGTCATTACTTCCTTGTACAAA |
| | | GGACATG-3′; SEQ ID NO: 154) |
| pET42T-hIL-1β₁₁₆₋₂₆₉ | hIL-1β₁₁₆₋₂₆₉ | SND-1 (5′-CTTTGTACAAGGAGAAGAAAAAATACCTGTGG |
| (ΔSND⁵²⁻⁵⁴) | (ΔSND⁵²⁻⁵⁴) | CCTTG-3′; SEQ ID NO: 155) |
| | (SEQ ID NO: 138) | SND-2 (5′-CAAGGCCACAGGTATTTTTTCTTCTCCTTGTA |
| | | CAAAG-3′; SEQ ID NO: 156) |
| pET42T-hIL-1β₁₁₆₋₂₆₉ | hIL-1β₁₁₆₋₂₆₉ | K6365S-1 (5′-GTGGCCTTGGGCCTCAGCGAAAGCAATCT |
| (K63S/K65S) | (K63S/K65S) | GTACCTGTCCTG-3′; SEQ ID NO: 157) |
| | (SEQ ID NO: 139) | K6365S-2 (5′-CAGGACAGGTACAGATTGCTTTCGCTGAG |
| | | GCCCAAGGCCAC-3′; SEQ ID NO: 158) |
| pET42T-hIL-1β₁₁₆₋₂₆₉ | hIL-1β₁₁₆₋₂₆₉ | QE-1 (5′-GTACATCAGCACCTCTGCAGCAGCAAACATGCC |
| (Q126A/E128A) | (Q126A/E128A) | CGTCTTC-3′; SEQ ID NO: 159) |
| | (SEQ ID NO: 140) | QE-2 (5′-GAAGACGGGCATGTTTGCTGCTGCAGAGGTGCT |
| | | GATGTAC-3′; SEQ ID NO: 160) |

### Example 8

### A. Biological Activity of Human IL-1β₁₁₆₋₂₆₉ and Human IL-1β₁₁₆₋₂₆₉ Muteins in Mice

Three female C3H/HeJ mice per group were injected intravenously with 10 μg of either the wild type human IL-1β₁₁₉₋₂₆₉ protein or one of the human IL-1β₁₁₉₋₂₆₉ protein muteins of EXAMPLE 7. Serum samples were withdrawn before and 3 h after injection and analysed for the relative increase in the concentration of the pro-inflammatory cytokine IL-6. As shown in Table 3, mice injected with the wild type human IL-1β₁₁₉₋₂₆₉ protein showed an increase of 2.38 ng/ml in serum IL-6 concentrations. With the exception of muteins hIL-1β₁₁₆₋₂₆₉ (D54R) and hIL-1β₁₁₆₋₂₆₉ (K63S/K65S), which induced similar serum IL-6 concentrations as wild type human IL-1β₁₁₉₋₂₆₉, all muteins tested induced lower amounts of IL-6, indicating reduced biological activity.

TABLE 3
Biological activity of human IL-1β₁₁₆₋₂₆₉ and human
IL-1β₁₁₆₋₂₆₉ muteins in mice.

| | Average increase in serum IL-6 |
|---|---|
| | concentrations 3 h after injection |
| Protein/mutein | in ng/ml (±SD) |
| hIL-1β₁₁₆₋₂₆₉ | 2.38 ± 0.69 |
| hIL-1β₁₁₆₋₂₆₉ (R4D) | 0.16 ± 0.03 |
| hIL-1β₁₁₆₋₂₆₉ (L6A) | 1.03 ± 0.65 |
| hIL-1β₁₁₆₋₂₆₉ (T9G) | 0.82 ± 0.42 |
| hIL-1β₁₁₆₋₂₆₉ (R11G) | 0.34 ± 0.25 |
| hIL-1β₁₁₆₋₂₆₉ (D54R) | 3.25 ± 1.67 |
| hIL-1β₁₁₆₋₂₆₉ (ΔEE^{50,51}) | 1.10 ± 0.27 |
| hIL-1β₁₁₆₋₂₆₉ (ΔSND⁵²⁻⁵⁴) | 0.13 ± 0.08 |
| hIL-1β₁₁₆₋₂₆₉ (K63S/K65S) | 2.22 ± 1.38 |
| hIL-1β₁₁₆₋₂₆₉ (Q126A/E128A) | 0.77 ± 0.55 |
| hIL-1β₁₁₆₋₂₆₉ (D145K) | 1.39 ± 0.26 |

### B. Biological Activity of Human IL-1β₁₁₆₋₂₆₉ and Human IL-1β₁₁₆₋₂₆₉ Muteins in Human PBMC

Peripheral blood mononuclear cells (PBMCs) were isolated from heparinized blood of a healthy donor by Ficoll density gradient centrifugation. 5×10⁵ cells per well were incubated with titrating amounts of either the wild type human IL-1β₁₁₉₋₂₆₉ protein or one of the human IL-1β₁₁₉₋₂₆₉ muteins of EXAMPLE 7. After over night incubation the amount of IL-6 in the cell culture supernatant was measured as readout of the biological activity. Table 4 shows that with the exception of muteins hIL-1β₁₁₆₋₂₆₉ (D54R) and hIL-1β₁₁₆₋₂₆₉ (K63S/K65S), much higher amounts of all mutants were necessary to induce the same IL-6 secretion as wild type human IL-1β₁₁₉₋₂₆₉, indicating a reduction in bioactivity. The factor by which biological activity was reduced ranged from 13 fold for mutein hIL-1β₁₁₆₋₂₆₉ (R11G) to 381 fold for mutein hIL-1β₁₁₆₋₂₆₉ (ΔSND⁵²⁻⁵⁴).

TABLE 4
Biological activity of human IL-1β₁₁₆₋₂₆₉ and human
IL-1β₁₁₆₋₂₆₉ muteins in human PBMC.

| | Protein/mutein | |
|---|---|---|
| | concentration | Fold |
| | (in ng/ml) | reduction in |
| | required to induce | bioactivity |
| | 600 pg/ml | relative to |
| | IL-6 from human | wild type |
| Protein/mutein | PBMC | hIL-1β₁₁₆₋₂₆₉ |
| hIL-1β₁₁₆₋₂₆₉ | 2 | —/— |
| hIL-1β₁₁₆₋₂₆₉ (R4D) | 333 | 146 |
| hIL-1β₁₁₆₋₂₆₉ (L6A) | 31 | 14 |
| hIL-1β₁₁₆₋₂₆₉ (T9G) | 79 | 34 |
| hIL-1β₁₁₆₋₂₆₉ (R11G) | 30 | 13 |
| hIL-1β₁₁₆₋₂₆₉ (D54R) | 5 | 2 |
| hIL-1β₁₁₆₋₂₆₉ (ΔEE^{50,51}) | 187 | 82 |
| hIL-1β₁₁₆₋₂₆₉ (ΔSND⁵²⁻⁵⁴) | 872 | 381 |
| hIL-1β₁₁₆₋₂₆₉ (K63S/K65S) | 13 | 6 |
| hIL-1β₁₁₆₋₂₆₉ (Q126A/E128A) | 94 | 41 |
| hIL-1β₁₁₆₋₂₆₉ (D145K) | 386 | 169 |

### Example 9

### A. Coupling of Human IL-1β₁₁₆₋₂₆₉ and Human IL-1β₁₁₆₋₂₆₉ Muteins to Qβ Virus-Like Particles

Chemical cross-linking of the wild type human IL-1β₁₁₉₋₂₆₉ protein and the human IL-1β₁₁₉₋₂₆₉ muteins of EXAMPLE 7 to Qβ virus-like particles was performed essentially as described in EXAMPLE 2A.

### B. Immunization of Mice with Human IL-1β₁₁₆₋₂₆₉ and Human IL-1β₁₁₆₋₂₆₉ Muteins Coupled to Qβ Capsid

Four female balb/c mice per group were immunized with Qβ coupled to either the wild type hIL-1β₁₁₆₋₂₆₉ protein or one of the hIL-1β₁₁₆₋₂₆₉ mutein proteins. Fifty μg of total protein were diluted in PBS to 200 μl and injected subcutaneously (100 μl on two ventral sides) on day 0, 14 and 28. Mice were bled retroorbitally on day 35, and sera were analyzed using ELISAs specific for either for the respective human IL-1β₁₁₆₋₂₆₉ mutein used as immunogen, or the wild type human IL-1β₁₁₆₋₂₆₉ protein.

### C ELISA

ELISA plates were coated either with the wild type hIL-1β₁₁₆₋₂₆₉ protein or the respective hIL-1β₁₁₆₋₂₆₉ mutein at a concentration of 1 μg/ml. The plates were blocked and then incubated with serially diluted mouse sera from day 35. Bound antibodies were detected with enzymatically labeled anti-mouse IgG antibody. Antibody titers of mouse sera were calculated as the average of those dilutions which led to half maximal optical density at 450 nm, and are shown in Table 5.

TABLE 5
Anti-hIL-1β₁₁₆₋₂₆₉ (wild type and mutein)-specific IgG titers
raised by immunization with Qβ-hIL-1β₁₁₆₋₂₆₉ or
Qβ-hIL-1β₁₁₆₋₂₆₉ mutein vaccines.

| | Average | Average |
|---|---|---|
| | anti-hIL-1β₁₁₆₋₂₆₉ | anti-hIL-1β₁₁₆₋₂₆₉ |
| | wild type IgG | mutein IgG titer |
| Vaccine | titer (±SD) | (±SD) |
| Qβ-hIL-1β₁₁₆₋₂₆₉ | 253325 ± 184813 | —/— |
| Qβ-hIL-1β₁₁₆₋₂₆₉ (R4D) | 231879 ± 115475 | 160666 ± 79478 |
| Qβ-hIL-1β₁₁₆₋₂₆₉ (L6A) | 120224 ± 7658 | 89377 + 17965 |
| Qβ-hIL-1β₁₁₆₋₂₆₉ (T9G) | 261249 ± 153716 | 224809 ± 131823 |
| Qβ-hIL-1β₁₁₆₋₂₆₉ (R11G) | 278342 ± 50296 | 279290 ± 47232 |
| Qβ-hIL-1β₁₁₆₋₂₆₉ (D54R) | 269807 ± 122351 | 206516 ± 90998 |
| Qβ-hIL-1β₁₁₆₋₂₆₉ (D145K) | 78365 ± 26983 | 93241 ± 28856 |
| Qβ-hIL-1β₁₁₆₋₂₆₉ (ΔEE^{50,51}) | 287625 ± 143835 | 229862 ± 140169 |
| Qβ-hIL-1β₁₁₆₋₂₆₉ (ΔSND⁵²⁻⁵⁴) | 68895 ± 14267 | 106116 ± 25295 |
| Qβ-hIL-1β₁₁₆₋₂₆₉ (K63S/K65S) | 403712 + 402594 | 244552 ± 173597 |
| Qβ-hIL-1β₁₁₆₋₂₆₉ (Q126A/E128A) | 195165 ± 71436 | 170434 ± 86831 |

Qβ-hIL-1β₁₁₆₋₂₆₉-immunization induced high titers of IgG antibodies against hIL-1β₁₁₆₋₂₆₉. Moreover, vaccination with either of the Qβ-hIL-1β₁₁₆₋₂₆₉ mutein vaccines induced high IgG titers against both the respective hIL-1β₁₁₆₋₂₆₉ mutein used as immunogen, and the wild type hIL-1β₁₁₆₋₂₆₉ protein.

### D. In Vitro Neutralization of Human IL-1β

Sera of mice immunized with Qβ coupled to either wild type hIL-1β₁₁₆₋₂₆₉ protein or to one of the hIL-1β₁₁₆₋₂₆₉ muteins were tested for their ability to inhibit the binding of human IL-1β protein to its receptor. ELISA plates were therefore coated with a recombinant human IL-1receptorI-hFc fusion protein at a concentration of 1 μg/ml, and co-incubated with serial dilutions of the above mentioned sera and 100 ng/ml of hIL-1β₁₁₆₋₂₆₉ protein. Binding of hIL-1β₁₁₆₋₂₆₉ to the immobilized human IL-1receptorI-hFc fusion protein was detected with a biotinylated anti-human IL-1β antibody and horse radish peroxidase conjugated streptavidin. All sera raised against Qβ-hIL-1β₁₁₆₋₂₆₉ mutein vaccines completely inhibited the binding of 100 ng/ml wild type hIL-1β₁₁₆₋₂₆₉ to hIL-1RI at serum concentrations ≧3.3%.

The same sera were also tested for their ability to inhibit the hIL-1β₁₁₆₋₂₆₉-induced secretion of IL-6 from human cells. Human PBMCs were therefore prepared as described in EXAMPLE 8B and incubated with 10 ng/ml wild type hIL-1β₁₁₆₋₂₆₉, which had been premixed with titrating concentrations of the sera described above. After over night incubation the cell culture supernatants were analyzed for the presence of IL-6. The neutralizing capacity of the sera was expressed as those dilutions which lead to half maximal inhibition of IL-6 secretion. In order to allow a direct comparison to the neutralizing capacity of the serum raised against wild type hIL-1β₁₁₆₋₂₆₉, the neutralizing titers of all sera raised against hIL-1β₁₁₆₋₂₆₉ muteins were corrected for the respective ELISA titers measured against wild type hIL-1β₁₁₆₋₂₆₉ (see Table 5). As shown in Table 6 all sera raised against hIL-1β₁₁₆₋₂₆₉ muteins were able to inhibit the secretion of IL-6 induced by wild type hIL-1β₁₁₆₋₂₆₉. The neutralizing titers ranged from 1:113 for sera raised against Qβ-hIL-1β₁₁₆₋₂₆₉ (R11G) to 1:4532 for sera raised against Qβ-hIL-1β₁₁₆₋₂₆₉ (D54R).

TABLE 6
Neutralizing titer determined in sera of mice immunized with
various IL-1 beta muteins.

| | Neutralizing titer |
|---|---|
| | (corrected for ELISA titer |
| Vaccine | against wild type hIL-1β₁₁₆₋₂₆₉) |
| Qβ-hIL-1β₁₁₆₋₂₆₉ | 3333 |
| Qβ-hIL-1β₁₁₆₋₂₆₉ (R4D) | 2150 |
| Qβ-hIL-1β₁₁₆₋₂₆₉ (L6A) | 2062 |
| Qβ-hIL-1β₁₁₆₋₂₆₉ (T9G) | 1036 |
| Qβ-hIL-1β₁₁₆₋₂₆₉ (R11G) | 113 |
| Qβ-hIL-1β₁₁₆₋₂₆₉ (D54R) | 4532 |
| Qβ-hIL-1β₁₁₆₋₂₆₉ (ΔEE^{50,51}) | 2871 |
| Qβ-hIL-1β₁₁₆₋₂₆₉ (ΔSND⁵²⁻⁵⁴) | 1109 |
| Qβ-hIL-1β₁₁₆₋₂₆₉ (K63S/K65S) | 3432 |
| Qβ-hIL-1β₁₁₆₋₂₆₉ (Q126A/E128A) | 1237 |
| Qβ-hIL-1β₁₁₆₋₂₆₉ (D145K) | 2369 |

### E. In Vivo Neutralization of IL-1β

The in vivo neutralizing capacity of the antibodies raised by immunization with Qβ coupled to either wild type hIL-1β₁₁₆₋₂₆₉ protein or to one of the hIL-1β₁₁₆₋₂₆₉ muteins is investigated. Three female C3H/HeJ mice per group are therefore immunized three times on days 0, 14, and 28 with 50 μg of either vaccine. On day 35 all immunized mice are injected intravenously with 1 μg of free wild type hIL-1β₁₁₆₋₂₆₉. As a control three naive mice are injected at the same time with the same amount of wild type hIL-1β₁₁₆₋₂₆₉. As readout of the inflammatory activity of the injected hIL-1β₁₁₆₋₂₆₉, serum samples are withdrawn immediately before and 3 h after injection and analysed for the relative increase in the concentration of the pro-inflammatory cytokine IL-6. Whereas naive mice show a strong increase in serum IL-6 concentrations 3 h after injection of hIL-1β₁₁₆₋₂₆₉, all mice immunized with Qβ coupled to the wild type hIL-1β₁₁₆₋₂₆₉ protein or to one of the hIL-1β₁₁₆₋₂₆₉ muteins do not show any increase in serum IL-6, indicating that the injected hIL-1β₁₁₆₋₂₆₉ is efficiently neutralized by the antibodies induced by the vaccines.

### Example 10

### A Cloning, Expression and Purification of Mouse IL-1α₁₁₅₋₂₇₀ and Mouse IL-1α_{115-270 (D}145K)

The Nucleotide Sequence Encoding Amino Acids 115-270 of Wild Type Murine IL-1α was amplified by PCR from a library of TNFα-activated murine macrophages using oligonucleotides IL1α1C (5′-ATATATCATA TGTCTGCCCC TTACACCTAC CAGAGTG-3′: SEQ ID NO:196) and IL1α2 (5′-ATATATCTCG AGTGATATCT GGAAGTCTGT CATAGAG-3′; SEQ ID NO:25). The DNA fragment was digested with NheI and XhoI, and cloned into the expression vector pET42T(+), giving rise to the expression plasmid pET42T-mIL-1α₁₁₅₋₂₇₀.

By site directed mutagenesis of the latter plasmid, an expression vector for the mutein mIL-1α₁₁₅₋₂₇₀ (D145K) was constructed. Using the oligonucleotide pair alphaD145K-1: (5′-GGACTGCCCTCTATGACAAAATTCCAGATATCACTCGAG-3; SEQ ID NO:197) alphaD145K-2 (5′-CTCGAGTGATATCTGGAATTTTGTCATAGAGGGCAGTCC-3′; SEQ ID NO:198) and the Quik-Change® Site directed mutagenesis kit (Stratagene), the D 145K mutation was introduced. Expression and purification of wild type mouse IL-1α₁₁₅₋₂₇₀ and the mutein mouse IL-1α₁₁₅₋₂₇₀(D145K) was performed as described in EXAMPLE 1.

### B Cloning, Expression and Purification of Human IL-1α₁₁₉₋₂₇₁ and Human IL-1β₁₁₉₋₂₇₁ (D145K)

The Nucleotide Sequence Encoding Amino Acids 119-271 of Wild Type Human IL-1α was amplified by PCR from a LPS-activated human B cell cDNA library using oligonucleotides HIL-3 (5′-ATATATCATA TGCTGAGCAA TGTGAAATAC AACTTTATG-3′; SEQ ID NO:141) and HIL-4 (5′-ATATATCTCG AGCGCCTGGT TTTCCAGTAT CTGAAAG-3′; SEQ ID NO:142). The DNA fragment was digested with NheI and XhoI, and cloned into the expression vector pET42T(+), giving rise to the expression plasmid pET42T-hIL-1β₁₁₉₋₂₇₁.

By site directed mutagenesis of the latter plasmid, an expression vector for the mutein hIL-1α₁₁₉₋₂₇₁ (D145K) was constructed. Using the oligonucleotide pair halphaD145K-1 (5′-GGGCCACCCT CTATCACTAA ATTTCAGATA CTGGAAAACC-3′: SEQ ID NO:199) and halphaD145K-2 (5′-GGTTTTCCAG TATCTGAAAT TTAGTGATAG AGGGTGGCCC-3′; SEQ ID NO:200) and the Quik-Change® Site directed mutagenesis kit (Stratagene), the D145K mutation was introduced. Expression and purification of wild type human IL-1α₁₁₉₋₂₇₁ and the human IL-1α₁₁₉₋₂₇₁ (D145K) mutein was performed as described in EXAMPLE 1.

### Example 11

### A. Biological Activity of Human IL-1α₁₁₉₋₂₇₁, Human IL-1α₁₁₉₋₂₇₁ (D145K), Mouse IL-1α₁₁₅₋₂₇₀, and Mouse IL-1α₁₁₅₋₂₇₀ (D145K) in Human PBMC

PBMC from a healthy donor (5×10⁵ cells per well) were incubated with titrating amounts of either the wild type human IL-1α₁₁₉₋₂₇₁ protein, the human IL-1α₁₁₉₋₂₇₁ (D145K) mutein, the wild type mouse IL-1α₁₁₅₋₂₇₀ protein, or the mouse IL-1α₁₁₅₋₂₇₀ (D145K) mutein. After over night incubation the amount of IL-6 in the cell culture supernatant was measured by Sandwich ELISA as readout of the biological activity of the different proteins. Table 9 shows that 21 fold higher amounts of the mouse IL-1α₁₁₅₋₂₇₀ (D145K) mutein were required to induce the same amount of IL-6 as the corresponding wild type mouse IL-1α₁₁₅₋₂₇₀ protein. In the case of the human IL-1α₁₁₉₋₂₇₁ (D145K) mutein 46-fold higher amounts than the wild type human IL-1α₁₁₉₋₂₇₁ protein were required. This demonstrates that both the human IL-1α₁₁₉₋₂₇₁ (D145K) mutein and the mouse IL-1α₁₁₅₋₂₇₀ (D145K) mutein have reduced bioactivity in human cells as compared to their wild type counterparts.

TABLE 7
Biological activity of IL-1α wild type proteins and muteins
in human PBMC as determined by IL-6 induction.

| Protein/mutein | Protein/mutein concentration |
|---|---|
| (expressed with SEQ ID | required to induce 600 pg/ml IL-6 |
| NO: 201 as C-terminal tag) | from human PBMC (ng/ml) |
| mouse IL-1α₁₁₅₋₂₇₀ | 4.7 |
| (SEQ ID NO: 202) | |
| mouse IL-1α₁₁₅₋₂₇₀ | 100 |
| (D145K) (SEQ ID NO: 204) | |
| human IL-1α₁₁₉₋₂₇₁ | 0.8 |
| (SEQ ID NO: 203) | |
| human IL-1α₁₁₉₋₂₇₁ (D145K) | 37 |
| (SEQ ID NO: 210) | |

### B. Biological Activity of Human IL-1α₁₁₉₋₂₇₁, Human IL-1α₁₁₉₋₂₇₁ (D145K), Mouse IL-1α₁₁₅₋₂₇₀ Protein, and Mouse IL-1α₁₁₅₋₂₇₀ (D145K) in Mice

Four female Balb/c mice per group were injected intravenously with 10 ng of either the wild type human IL-1α₁₁₉₋₂₇₁ protein, the human IL-1α₁₁₉₋₂₇₁ (D145K) mutein, the wild type mouse IL-1α₁₁₅₋₂₇₀ protein, or the mouse IL-1α₁₁₅₋₂₇₀ (D145K) mutein. Three hours after injection serum amyloid A (SAA) was measured in serum of injected mice as readout of the bioactivity of the respective protein. As shown in Table 8 the mouse IL-1α₁₁₅₋₂₇₀ (D145K) mutein induced 53% less SAA than the corresponding wild type mouse IL-1α₁₁₅₋₂₇₀ protein (p<0.05, Student t-test) and the human IL-1α₁₁₉₋₂₇₁ (D145K) mutein induced 67% less SAA than the corresponding wild type human IL-1α₁₁₉₋₂₇₁ protein (p<0.001 Student t-test). This demonstrates that both the human IL-1α₁₁₉₋₂₇₁ (D145K) mutein and the mouse IL-1α₁₁₅₋₂₇₀ (D145K) mutein have reduced bioactivity in mice when compared to their wild type counterparts.

TABLE 8
Biological activity of IL-1α wild type proteins and muteins
in mice determined by SAA.

| | Serum SAA concentration |
|---|---|
| | (μg/ml) 3 h after |
| Protein/mutein | protein/mutein injection (±SD) |
| mouse IL-1α₁₁₅₋₂₇₀ | 115 ± 32 |
| (SEQ ID NO: 202) | |
| mouse IL-1α₁₁₅₋₂₇₀ (D145K) | 55 ± 10 |
| (SEQ ID NO: 204) | |
| human IL-1α₁₁₉₋₂₇₁ | 92 ± 20 |
| (SEQ ID NO: 203) | |
| human IL-1α₁₁₉₋₂₇₁ (D145K) (SEQ ID | 31 ± 2 |
| NO: 210) | |

TABLE 9
Mouse IL-1 beta and Mouse IL-1 alpha muteins corresponding to
preferred human IL-1 beta muteins (SEQ ID NO: 131 to 140 and
SEQ ID NO: 205 to 209) and human IL-1 alpha muteins
(SEQ ID NO: 210 to 218) are created according to this table and
tested in the mouse model of rheumatoid arthritis.

| | Amino acid changes introduced |
|---|---|
| Human | in mouse IL-1beta 119-269 (SEQ |
| hIL-1beta 116-269 | ID NO: 164) in order to obtain |
| muteins | the corresponding mutation |
| R4D (SEQ ID NO: 131) | Exchange arginine at position 3 to aspartate |
| L6A (SEQ ID NO: 132) | Exchange leucine at position 5 to alanine |
| T9G (SEQ ID NO: 133) | Exchange arginine at position 8 to glycine |
| R11G(SEQ ID NO: 134) | Exchange arginine at position 10 to glycine |
| D54R (SEQ ID NO: 135) | Exchange aspartate at position 53 to arginine |
| D145K (SEQ ID NO: 136) | Exchange aspartate at position 143 to lysine |
| ΔEE50,51 | Delete glutamate, proline at positions 49, 50 |
| (SEQ ID NO: 137) | |
| ΔSND52-54 | Delete serine, asparagine, aspartate |
| (SEQ ID NO: 138) | at positions 51 to 53 |
| K63S/K65S | Exchange lysines at positions |
| (SEQ ID NO: 139) | 62 and 64 to serines |
| Q126A/E128A | Exchange glutamine at position |
| (SEQ ID NO: 140) | 125 to alanine and glutamate at position |
| | 127 to alanine |
| K88N (SEQ ID NO: 205) | Exchange lysine at position 87 to asparagine |
| R98Q (SEQ ID NO: 206) | Exchange arginine at position |
| | 97 to glutamine |
| K103L (SEQ ID NO: 207) | Exchange lysine at position 102 to leucine |
| ΔKKK92-94 | Delete lysine, lysine, lysine |
| (SEQ ID NO: 208) | at positions 91 to 93 |
| L10N (SEQ ID NO: 209) | Exchange leucine at position 9 to asparagine |
| | Amino acid changes introduced |
| Human | in mouse IL-1alpha 115-270 |
| hIL-1alpha 119-271 | (SEQ ID NO: 202) in order to obtain |
| muteins | corresponding mutation |
| D145K | Exchange aspartate at position 153 to lysine |
| (SEQ ID NO: 210) | |
| L18K (SEQ ID NO: 211) | Exchange methionine at position 25 to lysine |
| F146N | Exchange phenylalanine at position |
| (SEQ ID NO: 212) | 154 to asparagine |
| R10A (SEQ ID NO: 213) | Exchange lysine at position 17 to alanine |
| I62A (SEQ ID NO: 214) | Exchange tyrosine at position 70 to alanine |
| W107F | Exchange tryptophane at position |
| (SEQ ID NO: 215) | 115 to phenylalanine |
| D20V (SEQ ID NO: 216) | Exchange aspartate at position 27 to valine |
| ΔFIL16-18 | Delete phenylalanine, valine, methionine |
| (SEQ ID NO: 217) | at positions 23 to 25 |
| ΔITGS96-99 | Delete isoleucine, threonine, glycine, |
| (SEQ ID NO: 218) | serine at positions 104 to 107 |

### Example 12

### Amelioration of Diet-Induced Type II Diabetes in Male C57BL/6 Mice (Prophylactic Setting)

Male C57BL/6 mice were immunized on days 0, 14, and 28 with 50 μg of either Qβ, Qβ-mIL-1α₁₁₅₋₂₇₀, Qβ-mIL-1β₁₁₉₋₂₆₉ or a mixture of 50 μg Qβ-mIL-1β₁₁₅₋₂₇₀ and 50 μg Qβ-mIL-1β (n=16 per group). All mice were fed normal rodent chow (Provimi Kliba no. 3436: 18.5% protein, 4.5% fat, 4.5% fiber, 6.5% ash, 54% carbohydrates) during the immunization period. On day 35 this diet was replaced by a high fat diet (Provimi Kliba no. 2127: 23.9% protein, 35% fat, 4.9% fiber, 5% ash, 23.2% carbohydrates) for half of the mice of each group (n=8) while the other half (n=8) was kept on normal chow. Five months after the last immunization mice fed the high-fat diet were obese (average body weight>45 g) and showed elevated fasting glucose levels (Table 10, 0′).

In order to investigate on the diabetic phenotype of these mice an oral glucose tolerance test was performed by administering a dose of 2 mg/g body weight of D-glucose intragastrically and determining blood glucose levels at regular intervals using the Accu-check blood glucose meter (Roche). Table 10 shows that Qβ-immunized mice on normal chow showed an initial peak of 291.5 mg/dl in blood glucose levels after 15 minutes which was followed by a sharp drop and a complete return to pre-challenge levels within 90 minutes. The peak levels and kinetics of this response were essentially identical in all mouse groups that had been maintained on normal chow (Table 10). Qβ-immunized mice on high fat diet on the other hand peaked at higher levels (367.9 mg/dl) and failed to show a significant decline until 60 min post-challenge; only thereafter blood glucose levels started to decrease, without however returning to baseline levels within the 2 hour observation period. This severe impairment in glucose clearance indicates that obese Qβ-immunized mice had developed a diabetic phenotype. Obese Qβ-mIL-1α-, Qβ-mIL-1β-, or double immunized mice showed an initial increase in blood glucose levels to ˜350 mg/dl, which was immediately followed by a sustained decline, resulting in glucose levels that were consistently lower than in obese Qβ-immunized control mice. When calculating the area under the curves resulting form the repeated glucose measurements shown in table 10, it becomes evident, that obese Qβ-mIL-1α-, Qβ-mIL-1β-, or double immunized mice manifested an improved glucose clearance with respect to obese Qβ-immunized control mice (Table 11). Taken together these data show that immunization with Qβ-mIL-1α or Qβ-mIL-1β or a combination of both resulted in a clear amelioration of the diet-induced diabetic phenotype.

TABLE 10
Blood glucose levels (mg/dl; mean ± SEM) before and at different time points after intragastric
administration of 2 mg/g glucose. (Mice were fasted for a period of 5 hours before the experiment)

| Mouse group | 0′ | 15′ | 30′ | 45′ | 60′ | 90′ | 120′ |
|---|---|---|---|---|---|---|---|
| Qβ | 194.3 ± 8.0 | 342.4 ± 20.9 | 367.9 ± 28.3 | 356.9 ± 29.3 | 350.4 ± 31.6 | 298.5 ± 31.9 | 250.3 ± 23.3 |
| high fat diet | | | | | | | |
| Qβ-mIL-1α₁₁₅₋₂₇₀ | 195.6 ± 4.5 | 356.6 ± 7.4 | 324.3 ± 21.4 | 320.6 ± 26.9 | 303.3 ± 26.6 | 234.9 ± 19.6 | 208.6 ± 13.2 |
| high fat diet | | | | | | | |
| Qβ-mIL-1β₁₁₉₋₂₆₉ | 200.1 ± 11.6 | 341.9 ± 11.6 | 348.3 ± 27.6 | 313.3 ± 31.2 | 297.6 ± 27.3 | 256.9 ± 22.6 | 234.7 ± 19.0 |
| high fat diet | | | | | | | |
| Qβ-mIL-1α₁₁₅₋₂₇₀/Qβ-mIL-1β₁₁₉₋₂₆₉ | 190.6 ± 10.4 | 337.9 ± 27.1 | 309.9 ± 44.2 | 284.3 ± 47.6 | 281.6 ± 45.8 | 240.6 ± 40.8 | 221.0 ± 37.3 |
| high fat diet | | | | | | | |
| Qβ | 154.4 ± 3.5 | 291.5 ± 14.7 | 210.0 ± 10.0 | 193.6 ± 7.2 | 183.1 ± 6.2 | 149.5 ± 4.7 | 134.0 ± 4.2 |
| normal chow | | | | | | | |
| Qβ-mIL-1α₁₁₅₋₂₇₀ | 171.6 ± 4.8 | 297.4 ± 12.6 | 230.4 ± 10.9 | 203.0 ± 11.4 | 192.6 ± 11.7 | 158.0 ± 8.5 | 136.4 ± 6.2 |
| normal chow | | | | | | | |
| Qβ-mIL-1β₁₁₉₋₂₆₉ | 158.0 ± 3.6 | 312.3 ± 6.8 | 229.0 ± 10.8 | 188.3 ± 4.4 | 172.6 ± 4.9 | 145.8 ± 7.6 | 134.0 ± 3.8 |
| normal chow | | | | | | | |
| Qβ-mIL-1α₁₁₅₋₂₇₀/Qβ-mIL-1β₁₁₉₋₂₆₉ | 152.4 ± 4.4 | 283.6 ± 8.4 | 220.3 ± 11.3 | 188.5 ± 11.1 | 187.8 ± 9.0 | 149.1 ± 8.6 | 131.5 ± 6.9 |
| normal chow | | | | | | | |

TABLE 11
Glucose clearance in immunized mice. The area under the curve
(AUC) resulting from the consecutive glucose measurements
represented in Table 10 was calculated for each individual mouse.
Group means of AUC are expressed with SEM.

| Mouse group | Normal chow | High fat diet |
|---|---|---|
| Qβ | 4120 ± 460 | 14746 ± 2262 |
| Qβ-mIL-1α₁₁₅₋₂₇₀ | 6104 ± 2313 | 10184 ± 1800 |
| Qβ-mIL-1β₁₁₉₋₂₆₉ | 4276 ± 419 | 10459 ± 1699 |
| Qβ-mIL-1α₁₁₅₋₂₇₀/Qβ-mIL-1β₁₁₉₋₂₆₉ | 4464 ± 531 | 9500 ± 3382 |

### Example 13

### Amelioration of Diet-Induced Type II Diabetes in Male C57BL/6 Mice

The DNA sequence encoding amino acids 119-269 of mouse IL-1β was amplified by PCR from cDNA of TNFα-activated murine macrophages using oligonucleotides IL1BETA-3 (5′-ATATATGATATCCCCATTAGACAGCTGCACTACAGG-3; SEQ ID NO:226) and IL1BETA-2 5′-ATATATCTCGAGGGAAGACACAGATTCCATGGTGAAG-3′; SEQ ID NO:227) and cloned into the vector pET42T (EXAMPLE 7). The resulting plasmid pET42T-mIL-1β119-269 encodes the mature mouse IL-1β protein fused to a hexahistidine tag and a cysteine containing linker at the C-terminus. Due to the introduction of the EcoRV restriction site the valine residue at the N-terminus of mouse IL-1β is substituted by a short N-terminal extension consisting of three amino acids (MDI). By site directed mutagenesis of the plasmid pET42T-mIL-1β116-269, an expression vector was constructed which encodes the murine version of the human IL-1β mutein hIL-1β116-269 (D145K) (SEQ ID NO:136) with the C-terminal tag of SEQ ID NO:201, namely mIL-1β116-269 (D145K) (SEQ ID NO:228). The mutation was introduced using the Quik-Change® Site directed mutagenesis kit (Stratagene) and the oligonucleotides D143K-1 (5′-CAGTGGTCAG GACATAATTA AATTCACCAT GGAATCTGTGTC-3′; SEQ-ID:229) and D143K-2 (5′-GACACAGATT CCATGGTGAA TTTAATTATG TCCTGACCACTG-3′; SEQ ID NO:230). Expression and purification of the mutein mIL-1β116-269 (D145K) was performed as described in EXAMPLE 1 and coupling to Qβ was performed as described in EXAMPLE 2.

Groups of male C57BL/6 mice (8 weeks of age, n=8) were immunized subcutaneously on days 0, 14, 28, 42, and 147 with 50 μg of either Qβ or Qβ-mIL-1β119-269 (D145K). Starting with day 0, one half of the mice (n=16) was put on a high fat diet (Provimi Kliba no. 2127: 23.9% protein, 35% fat, 4.9% fiber, 5% ash, 23.2% carbohydrates), while the other half (n=16) was maintained on normal diet (Provimi Kliba no. 3436: 18.5% protein, 4.5% fat, 4.5% fiber, 6.5% ash, 54% carbohydrates) throughout the experiment. After eight months mice fed the high-fat diet were obese (average body weight>45 g) and showed elevated fasting glucose levels (Table 12).

In order to investigate on the diabetic phenotype of the mice on high fat diet an oral glucose tolerance test was performed by administering a dose of 2 mg/g body weight of D-glucose intragastrically and determining blood glucose levels at regular intervals using the Accu-check blood glucose meter (Roche). Table 13 shows that Qβ-immunized mice on high fat diet peaked at 318.5 mg/dl 30 minutes after injection and failed to show a significant decline until 60 min post-challenge; only thereafter blood glucose levels started to decrease, without however returning to baseline levels within the 2 hour observation period. This severe impairment in glucose clearance indicates that obese Qβ-immunized mice had developed a diabetic phenotype. Obese Qβ-mIL-1β119-269(D145K)-immunized mice showed an initial increase in blood glucose levels to 318.6 mg/dl, which was immediately followed by a sustained decline, resulting in glucose levels that were consistently lower than in obese Qβ-immunized control mice. Two hours after challenge blood glucose levels had returned to pre-challenge levels in these mice. When calculating the area under the curves resulting form the repeated glucose measurements shown in Table 13, it becomes evident, that obese Qβ-mIL-1β119-269 (D145K)-immunized mice manifested an improved glucose clearance with respect to obese Qβ-immunized control mice (Table 14). Taken together these data show that immunization with Qβ-mIL-1β119-269 (D145K) resulted in a clear amelioration of the diet-induced diabetic phenotype.

TABLE 12
Average body weights and fasting blood glucose levels after 5 hours
fasting (means ± SEM).

| | Average | Fasting blood |
|---|---|---|
| | body | glucose levels |
| | weight (g) | (mg/dl) |
| Qβ high fat diet | 47.16 ± 2.24 | 185.9 ± 6.3 |
| Qβ-mIL-1β₁₁₉₋₂₆₉ (D145K) high fat diet | 51.08 ± 1.23 | 194.0 ± 4.2 |
| Qβ normal chow | 36.95 ± 0.97 | 148.4 ± 6.5 |
| Qβ-mIL-1β₁₁₉₋₂₆₉ (D145K) normal chow | 36.23 ± 1.30 | 147.0 ± 3.1 |

TABLE 13
Blood glucose levels (mg/dl; mean ± SEM) before and at different time points after intragastric
administration of 2 mg/g glucose. Mice were fasted for a period of 5 hours before the experiment.

| Mouse group | 0′ | 15′ | 30′ | 45′ | 60′ | 90′ | 120′ |
|---|---|---|---|---|---|---|---|
| Qβ | 185.9 ± 6.3 | 315.1 ± 15.5 | 318.5 ± 23.1 | 305.5 ± 24.4 | 316.8 ± 33.5 | 238.4 ± 21.8 | 220.1 ± 15.3 |
| high fat diet | | | | | | | |
| Qβ-mIL-1β₁₁₉₋₂₆₉(D145K) | 194.0 ± 4.2 | 318.6 ± 18.3 | 290.0 ± 15.7 | 278.6 ± 12.5 | 280.1 ± 10.4 | 218.4 ± 7.8 | 199.4 ± 7.5 |
| high fat diet | | | | | | | |

TABLE 14
Glucose clearance in immunized mice. The area under the curve
(AUC) resulting from the consecutive glucose measurements
represented in Table 2 was calculated for each individual mouse.
Group means of AUC are expressed with SEM. Peaks below
baseline were excluded form the analysis.

| Mouse group | AUC |
|---|---|
| Qβ high fat | 11060 ± 1895 |
| Qβ-mIL-1β₁₁₉₋₂₆₉ (D145K) high fat | 7375 ± 539 |

1-24. (canceled)

25. A method of treating diabetes said method comprising administering an immunologically effective amount of a composition to an animal, said composition comprising:
(a) a virus-like particle (VLP) with at least one first attachment site; and
(b) at least one antigen with at least one second attachment site;
wherein said at least one antigen comprises an IL-1 molecule and wherein (a) and (b) are linked through said at least one first and said at least one second attachment site.

26. (canceled)

27. The method of claim 25, wherein said diabetes is type II diabetes.

28. The method of claim 25, wherein said animal is a human.

29. The method of claim 25, wherein said IL-1 molecule is an IL-1 beta molecule comprising an amino acid sequence selected from the group consisting of:
(a) human IL-1 beta 117-269 (SEQ ID NO:64);
(b) human IL-1 beta 116-269 (SEQ ID NO:165);
(c) mouse IL-1 beta 119-269s (SEQ ID NO:164); and
(d) an amino acid sequence which is at least 80% identical to any one of SEQ ID NO:64, SEQ ID NO:165, or SEQ ID NO:164.

30. The method of claim 25, wherein said IL-1 molecule is an IL-1 beta mutein, wherein said IL-1 beta mutein comprises a polypeptide having an amino acid sequence selected from SEQ ID NO:131 to SEQ ID NO:140 and SEQ ID NO:205 to SEQ ID NO:209.

31. The method of claim 25, wherein said IL-1 molecule is an IL-1 beta mutein, wherein said IL-1 beta mutein comprises the polypeptide of SEQ ID NO:136.

32. The method of claim 25, wherein said at least one antigen with at least one second attachment site is any one of SEQ ID NOs: 220 to 223.

33. The method of claim 25, wherein said at least one antigen with at least one second attachment site is SEQ ID NO:220.

34. The method of claim 25, wherein said IL-1 molecule is an IL-1 alpha molecule comprising an amino acid sequence selected from the group consisting of:
(a) human IL-1 alpha 119-271 (SEQ ID NO:63);
(b) human IL-1 alpha 119-271 (SEQ ID NO:203);
(c) mouse IL-1 alpha 117-270 (SEQ ID NO:163); and
(d) an amino acid sequence which is at least 80% identical to any one of SEQ ID NO:63, SEQ ID NO:163, or SEQ ID NO:203.

35. The method of claim 25, wherein said virus-like particle is a virus-like particle of an RNA bacteriophage.

36. The method of claim 25, wherein said virus-like particle is a virus-like particle of RNA bacteriophage Qβ.

37. The method of claim 25, wherein said virus-like particle comprises recombinant coat proteins, mutants or fragments thereof, of an RNA bacteriophage.

38. The method of claim 25, wherein said virus-like particle comprises recombinant coat proteins, wherein said recombinant coat proteins comprise or consist of SEQ ID NO:3.

39. The method of claim 25, wherein said first attachment site is linked to said second attachment site via at least one non-peptide covalent bond.

40. The method of claim 25, wherein said first attachment is an amino group of a lysine, and wherein said second attachment site is a sulfhydryl group of a cysteine.

41. The method of claim 25, wherein only one of said second attachment sites associates with said first attachment site through at least one non-peptide covalent bond leading to a single and uniform type of binding of said antigen to said virus-like particle, wherein said only one second attachment site that associates with said first attachment site is a sulfhydryl group, and wherein said antigen and said virus-like particle interact through said association to form an ordered and repetitive antigen array.

42. The method of claim 25, wherein said first attachment site is linked to said second attachment site via at least one peptide bond, and wherein said virus-like particle comprises recombinant coat proteins, mutants or fragments thereof, of an RNA bacteriophage, and wherein said at least one antigen is fused to the N- or the C-terminus of said recombinant coat proteins, mutants or fragments thereof.

43. A method of treating diabetes, said method comprising administering an immunologically effective amount of a vaccine to an animal, wherein said vaccine comprises a composition comprising:
(a) a virus-like particle (VLP) with at least one first attachment site; and
(b) at least one antigen with at least one second attachment site;
wherein said at least one antigen comprises an IL-1 molecule and wherein (a) and (b) are linked through said at least one first and said at least one second attachment site.

44. The method of claim 43, wherein said vaccine comprises:
(i) a first composition, wherein said first composition comprises:
(a) a virus-like particle (VLP) with at least one first attachment site; and
(b) at least one antigen with at least one second attachment site;
wherein said at least one antigen comprises an IL-1 beta molecule and wherein (a) and (b) are linked through said at least one first and said at least one second attachment site; and
(ii) a second composition, wherein said second composition comprises:
(a) a virus-like particle (VLP) with at least one first attachment site; and
(b) at least one antigen with at least one second attachment site;
wherein said at least one antigen comprises an IL-1 alpha molecule and wherein (a) and (b) are linked through said at least one first and said at least one second attachment site.

45. The method of claim 44, wherein said IL-1 beta molecule is SEQ ID NO:136 or SEQ ID NO:165, and/or wherein said IL-1 alpha molecule is SEQ ID NO:203 or SEQ ID NO:210.
